SEQUENCE LISTING (18 sequences)

SEQ ID NO: 1 - DNA, 32 bp, synthetic construct
  misc_feature 1..32: note = Portion of lentiviral vector
  source 1..32: mol_type = other DNA
atctcgacacaaatggcagtattcatccacaa

SEQ ID NO: 2 - DNA, 12 bp, synthetic construct
  misc_feature 1..12: note = Portion of lentiviral vector
  source 1..12: mol_type = other DNA
atcggtacgtac

SEQ ID NO: 3 - DNA, 24 bp, synthetic construct
  misc_feature 1..24: note = Portion of lentiviral vector
  source 1..24: mol_type = other DNA
cgggtttattacagggacagcaga

SEQ ID NO: 4 - DNA, 425 bp, synthetic construct
  misc_feature 1..425: note = Reduced length hypersensitive site EC2
  source 1..425: mol_type = other DNA
tacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctt

SEQ ID NO: 5 - DNA, 344 bp, synthetic construct
  misc_feature 1..344: note = Reduced length hypersensitive site EC3
  source 1..344: mol_type = other DNA
tgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcc

SEQ ID NO: 6 - DNA, 410 bp, synthetic construct
  misc_feature 1..410: note = Reduced length hypersensitive site EC4
  source 1..410: mol_type = other DNA
caggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttat

SEQ ID NO: 7 - DNA, 634 bp, synthetic construct
  misc_feature 1..634: note = Reduced length hypersensitive site EC1
  source 1..634: mol_type = other DNA
catcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgaggg

SEQ ID NO: 8 - DNA, 193 bp, synthetic construct
  misc_feature 1..193: note = BCL11A shmiR in miR223 Fwd orientation
  source 1..193: mol_type = other DNA
gatctcacttccccacagaagctcttggcctggcctcctgcagtgccacgctgcgcgatcgagtgttgaataactccatgtggtagagttattcaacactcgatcgcgcagtgcggcacatgcttaccagctctaggccagggcagatgggatatgacgaatggactgccagctggatacaaggatgctcacc

SEQ ID NO: 9 - DNA, 193 bp, synthetic construct
  misc_feature 1..193: note = BCL11A shmiR in miR223 Rev complement
  source 1..193: mol_type = other DNA
ggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatc

SEQ ID NO: 10 - DNA, 154 bp, synthetic construct
  misc_feature 1..154: note = ZNF410 shmiR in miR144 Fwd. Orientation
  source 1..154: mol_type = other DNA
cgcttttcaagccatgcttcctgtgcccccagtggggccctggctggctgagcacttagtgtttgtaagtttgcgatgagacactacaaacactaagtgctcagcagtccgggcacccccagctctggagcctgacaaggaggacaggagagat

SEQ ID NO: 11 - DNA, 154 bp, synthetic construct
  misc_feature 1..154: note = ZNF410 shmiR in miR144 Rev complement
  source 1..154: mol_type = other DNA
atctctcctgtcctccttgtcaggctccagagctgggggtgcccggactgctgagcacttagtgtttgtagtgtctcatcgcaaacttacaaacactaagtgctcagccagccagggccccactgggggcacaggaagcatggcttgaaaagcg

SEQ ID NO: 12 - DNA, 9555 bp, synthetic construct
  misc_feature 1..9555: note = Lentiviral vector
  source 1..9555: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagagggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcgtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccatcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgagggtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 13 - DNA, 9555 bp, synthetic construct
  misc_feature 1..9555: note = Lentiviral vector
  source 1..9555: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattgggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcgtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccatcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgagggtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 14 - DNA, 9555 bp, synthetic construct
  misc_feature 1..9555: note = Lentiviral vector
  source 1..9555: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaataggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcgaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccatcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgagggtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 15 - DNA, 9555 bp, synthetic construct
  misc_feature 1..9555: note = Lentiviral vector
  source 1..9555: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccatcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgagggtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 16 - DNA, 9555 bp, synthetic construct
  misc_feature 1..9555: note = Lentiviral vector
  source 1..9555: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatccccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccatcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgagggtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 17 - DNA, 9731 bp, synthetic construct
  misc_feature 1..9731: note = Lentiviral vector
  source 1..9731: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagagatctctcctgtcctccttgtcaggctccagagctgggggtgcccggactgctgagcacttagtgtttgtagtgtctcatcgcaaacttacaaacactaagtgctcagccagccagggccccactgggggcacaggaagcatggcttgaaaagcgacctcgaggacgcgtcgacgtcggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcgtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccatcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgagggtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 18 - DNA, 9731 bp, synthetic construct
  misc_feature 1..9731: note = Lentiviral vector
  source 1..9731: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagacatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataatctctcctgtcctccttgtcaggctccagagctgggggtgcccggactgctgagcacttagtgtttgtagtgtctcatcgcaaacttacaaacactaagtgctcagccagccagggccccactgggggcacaggaagcatggcttgaaaagcgacctcgaggacgcgtcgacgtcggtgagcatccttgtatccagctggcagtccattcgtcatatcccatctgccctggcctagagctggtaagcatgtgccgcactgcgcgatcgagtgttgaataactctaccacatggagttattcaacactcgatcgcgcagcgtggcactgcaggaggccaggccaagagcttctgtggggaagtgagatcccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttaccatcaataattctagccccacaggagtttgttctgaaagtaaacttccacaaccgcaagcttattgaggctaaggcatctgtgaaggaaagaaacatctcctctaaaccactatgctgctagagcctcttttctgtactcaagcctcattcagacactagtgtcaccagtctcctcatatacctattgtattttcttcttcttgctggtttagtcatgttttctgggagcttaggggcttattttattttgttttgttttctaatcaacagagatgggcaaacccattatttttttctttagacttgggatggtgatagctgggcagcgtcagaaactgtgtgtggatatagataagagctcaggactatgctgagctgtgatgagggaggggcctagctaaaggcagtgagagtcagaatgctcctgctattgccttctcagtccccacgcttggtttctacacaagtagatacatagaaaaggctataggttagtgtttgagagtcctgcatgattagttgctcagaaatgcccgataaatatgttatgtgtgtttatgtatatatatgttttatatgtgtgtgtgtgtgtgttgtgtttacaaatatgtgattatcatcaaaacgtgagggtacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagctttgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcccaggcttggattcaaagctcctgactttctgtctagtgtatgtgcagtgagccccttttcctctaactgaaagaaggaaaaaaaaatggaacccaaaatattctacatagtttccatgtcacagccagggctgggcagtctcctgttatttcttttaaaataaatatatcatttaaatgcataaataagcaaaccctgctcgggaatgggagggagagtctctggagtccaccccttctcggccctggctctgcagatagtgctatcaaagccctgacagagccctgcccattgctgggccttggagtgagtcagcctagtagagaggcagggcaagccatctcatagctgctgagtgggagagagaaaagggctcattgtctataaactcaggtcatggctattcttattaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

(19) United States
(12) Patent Application Publication
(10) Pub. No.: US 2025/0235480 A1
(43) Pub. Date: Jul. 24, 2025

(54) VECTORS COMBINING ANTI-SICKLING BETA-AS3-GLOBIN WITH ANTI BCEL11A SHRNAMIR TO TREAT BETA-HEMOGLOBINOPATHIES

(71) Applicants: The Regents of the University of California, Oakland, CA (US); The Children's Medical Center Corporation, Boston, MA (US); Dana-Farber Cancer Institute, Inc., Boston, MA (US)

(72) Inventors: Donald B. KOHN, Tarzana, CA (US); Roger HOLLIS, Los Angeles, CA (US); Kevyn HART, Los Angeles, CA (US); David WILLIAMS, Midland, MI (US); Christian BRENDEL, Boston, MA (US); Boya LIU, Boston, MA (US)

(73) Assignees: The Regents of the University of California, Oakland, CA (US); The Children's Medical Center Corporation, Boston, MA (US); Dana-Farber Cancer Institute, Inc., Boston, MA (US)

(21) Appl. No.: 18/845,136

(22) PCT Filed: Mar. 10, 2023
(86) PCT No.: PCT/US23/64191

§ 371 Date: Sep. 9, 2024

Related U.S. Application Data
(60) Provisional application No. 63/319,152, filed on Mar. 11, 2022.

(51) Int. Cl. A61K 35/28; A61P 7/00; C07K 14/805; C12N 5/071; C12N 5/0775; C12N 15/113; C12N 15/86

(52) U.S. Cl. CPC A61K 35/28; A61P 7/00; C07K 14/805; C12N 5/0663; C12N 5/0686; C12N 15/113; C12N 15/86; C12N 2310/141; C12N 2310/531; C12N 2740/15043

(57) ABSTRACT

In certain embodiments, a lentiviral vector for the treatment of sickle cell disease (SCD) is provided. In certain embodiments, the vector comprises an expression cassette that encodes that an anti-sickling β-globin gene and an shRNA that inhibits expression of a BCL11A gene (BCL11A shRNA) wherein said expression cassette is in reverse orientation in the vector; a β-globin locus control region (LCR) comprising a reduced length hypersensitive site 1 (HS1) sequence, a reduced length hypersensitive site 2 (HS2) sequence, a reduced length hypersensitive site 3 (HS3) sequence, and a reduced length hypersensitive site 4 (HS4) sequence, where said anti-sickling β-globin gene is operably linked to the human β-globin locus control region.

### CROSS-REFERENCE TO RELATED APPLICATIONS

This application is a National Phase Application of PCT International Application No. PCT/US23/64191, International Filing Date Mar. 10, 2023, which claims benefit of and priority to U.S. Ser. No. 63/319,152, filed on Mar. 11, 2022, which are incorporated herein by reference in their entirety for all purposes.

### SEQUENCE LISTING STATEMENT

This application contains references to nucleic acid sequences that have been submitted concurrently herewith as the sequence listing ST26 format XML file “UCLAP241WO.XML”, file size 93,322 bytes, created on Mar. 7, 2023, which is incorporated by reference in its entirety pursuant to 37 C.F.R. § 1.52(e)(5).

### BACKGROUND

Sickle cell disease (SCD) is one of the most common monogenic disorders worldwide and is a major cause of morbidity and early mortality (Hoffman et al. (2009) Hematology: Basic Principles and Practice. 5th ed. London, United Kingdom, Churchill Livingstone). SCD affects approximately 80,000-100,000 Americans, and causes significant neurologic, pulmonary, and renal injury, as well as severe acute and chronic pain that adversely impacts quality of life. It is estimated that approximately 240,000 children are born annually in Africa with SCD and 80% die by their second birthday. The average lifespan of subjects with SCD in the United States is approximately 40-45 years and this has remained unchanged over the last 3-4 decades.

SCD is caused by a single amino acid change in β-globin (Glu 6 to Val 6) which leads to hemoglobin polymerization and red blood cell (rbc) sickling. SCD typically results in continual low-grade ischemia and episodic exacerbations or “crises” resulting in tissue ischemia, organ damage, and premature death.

Although SCD is well characterized, there is still no ideal long-term treatment. Current therapies are based on induction of fetal hemoglobin (HbF) to inhibit polymerization of sickle hemoglobin (HbS) (Voskaridou et al. (2010) Blood, 115(12): 2354-2363) and cell dehydration (Eaton and Hofrichter (1987) Blood, 70(5): 1245-1266) or reduction of the percentage of HbS by transfusions (Stamatoyannopoulos et al., eds. (2001) Molecular Basis of Blood Diseases. 3rd ed. Philadelphia, Pennsylvania, USA: WB Saunders). Allogeneic human stem cell transplantation (HSCT) from bone marrow (BM) or umbilical cord blood (UCB) or mobilized peripheral blood stem cells (mPBSC) is a potentially curative therapy, although only a small percentage of patients have undergone this procedure, mostly children with severe symptoms who had HLA-matched sibling donors (Bolaños-Meade and Brodsky (2009) Curr. Opin. Oncol. 21(2): 158-161; Rees et al. (2010) Lancet, 376(9757): 2018-2031; Shenoy (2011) Hematology Am Soc Hematol Educ Program. 2011: 273-279).

Transplantation of allogeneic cells carries the risk of graft-versus host disease (GvHD), which can be a cause of extensive morbidity. HSCT using UCB from matched unrelated donors holds reduced risk of acute or chronic GvHD compared with using BM; however, there is a higher probability of engraftment failure using UCB as a result of its lower cell dose and immunologic immaturity (Kamani et al. (2012) Biol. Blood Marrow Transplant. 18(8): 1265-1272; Locatelli and Pagliara (2012) Pediatr. Blood Cancer. 59(2): 372-376).

Gene therapy with autologous human stem cells (HSCs) is an alternative to allogeneic HSCT, since it avoids the limitations of finding a matched donor and the risks of GvHD and graft rejection. For gene therapy application in SCD patients, one source for autologous HSC would be BM, due to the complications previously described when G-CSF was used to collect autologous peripheral blood stem cells (PBSCs) in SCD patients (Abboud et al. (1998) Lancet 351(9107): 959; Adler et al. (2001) Blood, 97(10): 3313-3314; Fitzhugh et al. (2009) Cytotherapy, 11(4): 464-471). However, more recently, plerixafor, an immunostimulat, can be used to mobilize hematopoietic stem cells into the blood stream (Esrick et al. (2018) Blood Adv. 2(19): 2505-2512). The stem cells can then be extracted from the blood and use. Although general anesthesia imposes a risk for SCD patients as well, current best medical practices can minimize these (Neumayr et al. (1998) Am. J. Hematol. 57(2): 101-108).

The development of integrating vectors for β-globin gene transfer has been challenging due to the complex regulatory elements needed for high-level, erythroid-specific expression (Lisowski a& Sadelain (2008) Br. J. Haematol. 141(3): 335-345). γ-Retroviral vectors were unable to transfer these β-globin expression cassettes intact (Gelinas et al. (1989) Adv. Exp. Med. Biol. 271: 135-148; Gelinas et al. (1989) Prog. Clin. Biol. Res. 316B: 235-249). In contrast, lentiviral vectors (LV) can transfer β-globin cassettes intact with relatively high efficiency, although the titers of these vectors are reduced compared with those of vectors bearing simpler cassettes (see, e.g., May et al. (2000) Nature 406(6791): 82-86; Pawliuk et al. (2001) Science, 294(5550): 2368-2371). In the last decade, many groups have developed different β-globin LV for targeting β-hemoglobinopathies, with successful therapeutic results following transplantation of ex vivo-modified HSC in mouse models (May et al. (2000) Nature 406(6791): 82-86; Pawliuk et al. (2001) Science, 294(5550): 2368-2371; Levasseur et al. (2003) Blood, 102(13):4312-4319; Hanawa et al. (2004) Blood, 104(8): 2281-2290; Puthenveetil et al. (2004) Blood, 104(12): 3445-3453; Miccio et al. (2008) Proc. Natl. Acad. Sci. USA, 105(30):10547-10552; Pestina et al. (2008) Mol. Ther. 17(2): 245-252; Morgan et al. (2020) Mol. Ther. 28(1): 328-340; Esrick et al. (2021) N. Engl. J. Med., 384: 205-215). Recently, Bluebird Bio, updated trial results with longer follow-up from lentiviral vector treated patients with beta-thalassemia and sickle cell disease. Across three small studies testing the gene therapy in the two blood diseases, patients given LentiGlobin saw their levels of the crucial oxygen-carrying protein hemoglobin rise to approach normal, eliminating the need for blood transfusions in most over the studied period.

Sickle patients with hereditary persistence of fetal hemoglobin (HbF) (HPFH) have improved survival and amelioration of clinical symptoms, with maximal clinical benefits observed when the HbF is elevated above threshold values (e.g., 8%-15% of the total cellular Hb) (Voskaridou et al. (2010) Blood, 115(12): 2354-2363; Platt et al. (1994) N. Engl. J. Med. 330(23): 1639-1644). Therefore, some gene therapy strategies have employed viral vectors carrying the human γ-globin gene (HBG1/2). However, these constructs expressed HbF poorly in adult erythroid cells, since fetal-specific transcription factors are required for high-level expression of the γ-globin gene (Chakalova et al. (2005) Blood 105(5): 2154-2160; Russell (2007) Eur. J. Haematol. 79(6): 516-525). These limitations have been overcome by embedding the exons encoding human γ-globin within the human β-globin gene 5′ promoter and 3′ enhancer elements (Hanawa et al. (2004) Blood, 104(8): 2281-2290; Persons et al. (2002) Blood, 101(6): 2175-2183; Perumbeti et al. (2009) Blood, 114(6): 1174-1185). Breda et al. (2012) PLoS One, 7(3): e32345 used an LV vector encoding the human hemoglobin (HBB) gene to increase the expression of normal HbA in CD34⁺-derived erythroid cells from SCD patients, however, the expression level needed when the HBB gene is used would be higher than would be required for HBG1/2 gene expression to achieve therapeutic benefits in SCD patients.

Another approach is to modify β-globin genes to confer antisickling activity by substituting key amino acids from γ-globin. The modified β-globin cassette should yield the necessary high-level, erythroid-specific expression in adult erythroid cells. Pawliuk et al. (2001) Science, 294(5550): 2368-2371 designed an LV carrying a human β-globin gene with the amino acid modification T87Q. The glutamine at position 87 of γ-globin has been implicated in the anti-sickling activity of HbF (Nagel et al. (1979) Proc. Natl. Acad. Sci., USA, 76(2): 670-672). This anti-sickling construct corrected SCD in 2 murine models of the disease, and a similar LV has been used in a clinical trial for β-thalassemia and SCD in France (Cavazzana-Calvo et al. (2010) Nature, 467(7313): 318-322). The vector used by Bluebird for the clinical trials discussed above carries a beta-globin gene with the T87Q substitution,

Townes and colleagues have taken a similar approach, developing a recombinant human anti-sickling β-globin gene (HBBAS3) encoding a β-globin protein (HbAS3) that has 3 amino substitutions compared with the original (HbA): T87Q for blocking the lateral contact with the canonical Val 6 of HbS, E22A to disrupt axial contacts (McCune et al. (1994) Proc. Natl. Acad. Sci. USA, 91(21): 9852-9856) and G16D, which confers a competitive advantage over sickle-β-globin chains for interaction with the α-globin polypeptide (Vandenesch et al. (1987) Clin. Chim. Acta. 168(2):121-128). Functional analysis of the purified HbAS3 protein demonstrated that this recombinant protein had potent activity to inhibit HbS tetramer polymerization (Levasseur et al. (2004) J. Biol. Chem. 279(26): 27518-27524.). Levasseur et al. (2003) Blood, 102(13): 4312-4319, showed efficient transduction of BM stem cells from a murine model of SCD with a self-inactivating (SIN) LV carrying the HBBAS3 transgene that resulted in normalized rbc physiology and prevented the pathological manifestations of SCD.

Unfortunately, various β-globin expression vectors, suffer from low vector titer and/or sub-optimal gene transfer to hematopoietic stem cells, and/or less than desirable β-globin expression, representing limitations impacting the effective implementation of this gene therapy strategy to the clinic.

### SUMMARY

Various embodiments provided herein may include, but need not be limited to, one or more of the following:

Embodiment 1: A recombinant lentiviral vector (LV) comprising:
an expression cassette that encodes that an anti-sickling β-globin gene wherein said expression cassette is in reverse orientation in said vector;
a β-globin locus control region (LCR) comprising a reduced length hypersensitive site 2 (HS2) sequence, a reduced length hypersensitive site 3 (HS3) sequence, and a reduced length hypersensitive site 4 (HS4) sequence, where said anti-sickling β-globin gene is operably linked to said human β-globin locus control region; and
an shRNA in a microRNA scaffold (shmiR), that inhibits expression of the BCL11A gene (BCL11A shmiR) and/or an shRNA in a microRNA scaffold (shmiR), that inhibits expression of a ZNF410 gene (ZNF410 shmiR).

Embodiment 2: The lentiviral vector of embodiment 1, wherein said vector comprises a globin promoter.

Embodiment 3: The lentiviral vector according to any one of embodiments 1-2, wherein said vector comprises a BCL11A shmiR.

Embodiment 4: The lentiviral vector according to any one of embodiments 1-3, wherein said reduced length hypersensitive site 2 (HS2) sequence consists of the nucleotide sequence of EC2 (SEQ ID NO:4).

Embodiment 5: The lentiviral vector according to any one of embodiments 1-4, wherein said reduced length hypersensitive site 3 (HS3) sequence consists of the nucleotide sequence of EC3 (SEQ ID NO:5).

Embodiment 6: The lentiviral vector according to any one of embodiments 1-5, wherein said reduced length hypersensitive site 4 (HS4) sequence consists of the nucleotide sequence of EC4 (SEQ ID NO:6).

Embodiment 7: The lentiviral vector according to any one of embodiments 1-6, wherein said vector comprises a reduced length hypersensitive site 1 (HS1) sequence.

Embodiment 8: The lentiviral vector of embodiment 1-7, wherein said reduced length hypersensitive site 1 (HS1) sequence consists of the nucleotide sequence of EC1 (SEQ ID NO:7).

Embodiment 9: The lentiviral vector according to any one of embodiments 1-8, wherein said anti-sickling human beta globin gene encoding an anti-sickling-beta globin polypeptide comprise one or more mutations selected from the group consisting of Gly16Asp, Glu22Ala and Thr87Gln.

Embodiment 10: The lentiviral vector of embodiment 9, wherein said beta globin gene comprises the mutation Gly16Asp.

Embodiment 11: The lentiviral vector according to any one of embodiments 9-10, wherein said beta globin gene comprises the mutation Glu22Ala.

Embodiment 12: The lentiviral vector according to any one of embodiments 9-11, wherein said beta globin gene comprises the mutation Thr87Gln.

Embodiment 13: The lentiviral vector of embodiment 9, wherein said anti-sickling human β-globin gene comprises about 2.3 kb of recombinant human β-globin gene including exons and introns under the control of said human β-globin locus control region.

Embodiment 14: The lentiviral vector according to any one of embodiments 1-13, wherein said β-globin gene comprises PAS3 comprising an intervening sequence I (IVS1) and an intervening sequence 2 (JVS).

Embodiment 15: The lentiviral vector of embodiment 14, wherein said (3-globin gene comprises β-globin intron 2 with a 375 bp RsaI deletion from IVS2.

Embodiment 16: The lentiviral vector embodiment 14, wherein said β-globin gene comprises an SspI (S) to RsaI (R) deletion (˜220 bp).

Embodiment 17: The lentiviral vector embodiment 14, wherein said β-globin gene comprises a 591 bp deletion in IVS2.

Embodiment 18: The lentiviral vector according to any one of embodiments 1-17, wherein said BCL11A shmiR comprises a BCL11A shRNA embedded in a microRNA sequence.

Embodiment 19: The lentiviral vector of embodiment 18, wherein said BCL11A shRNA is embedded in microRNA 223 (MIR223).

Embodiment 20: The lentiviral vector of embodiment 19, wherein said BCL11A shmiR comprises the nucleotide sequence

(SEQ ID NO: 8)
GATCTCACTTCCCCACAGAAGCTCTTGGCCTGGCCTCCTGCAGTGCCAC
GCTGCGCGATCGAGTGTTGAATAActccatgtggtagagTTATTCAACA
CTCGATCGCGCAGTGCGGCACATGCTTACCAGCTCTAGGCCAGGGCAGA
TGGGATATGACGAATGGACTGCCAGCTGGATACAAGGATGCTCACC.

Embodiment 21: The lentiviral vector according to any one of embodiments 1-20, wherein said BCL11A shmiR is located upstream or downstream of said anti-sickling (3-globin gene.

Embodiment 22: The lentiviral vector according to any one of embodiments 1-20, wherein said BCL11A shmiR is disposed in a noncoding sequence of said anti-sickling β-globin gene.

Embodiment 23: The lentiviral vector according to any one of embodiments 14-20, wherein said BCL11A shmiR is disposed in IVS1 and/or IVS2.

Embodiment 24: The lentiviral vector of embodiment 23, wherein said BCL11A shmiR is disposed in IVS1.

Embodiment 25: The lentiviral vector of embodiment 24, wherein said BCL11A shmiR is disposed in the upstream half or quarter of IVS1.

Embodiment 26: The lentiviral vector of embodiment 24, wherein said BCL11A shmiR is disposed in the downstream half or quarter of IVS1.

Embodiment 27: The lentiviral vector of embodiment 23, wherein said BCL11A shmiR is disposed in IVS2.

Embodiment 28: The lentiviral vector of embodiment 27, wherein said BCL11A shmiR is disposed in the upstream half or quarter of IVS2.

Embodiment 29: The lentiviral vector of embodiment 27, wherein said BCL11A shmiR is disposed in the downstream half or quarter of IVS2.

Embodiment 30: The lentiviral vector of embodiment 27, wherein said BCL11A shmiR is disposed at or downstream from a deletion in IVS2.

Embodiment 31: The lentiviral vector according to any one of embodiments 1-20, wherein said vector comprises features shown and located as in the vector map in FIG. 2 and/or the feature list in FIG. 3.

Embodiment 32: The lentiviral vector of embodiment 1, wherein said vector comprises the nucleic acid sequence shown in Table 4 (SEQ ID NO:12).

Embodiment 33: The lentiviral vector of embodiment 1, wherein said vector comprises the nucleic acid sequence shown in Table 5 (SEQ ID NO:13).

Embodiment 34: The lentiviral vector of embodiment 1, wherein said vector comprises the nucleic acid sequence shown in Table 6 (SEQ ID NO:14).

Embodiment 35: The lentiviral vector of embodiment 1, wherein said vector comprises the nucleic acid sequence shown in Table 7 (SEQ ID NO: 15).

Embodiment 36: The lentiviral vector according to any one of embodiments 1-30, wherein said vector comprises a ZNF410 shRNA).

Embodiment 37: The lentiviral vector of embodiment 36, wherein said ZNF410 shmiR comprises a ZNF410 shRNA embedded in microRNA 144 (MIR144).

38: The lentiviral vector of embodiment 37, wherein said ZNF410 shmiR comprises the nucleotide sequence

(SEQ ID NO: 10)
cgcttttcaagccatgcttcctgtgcccccagtggggccctggctgGCT
GAGCACTTAGTGTTTGTAagtttgcgatgagacacTACAAACACTAAGT
GCTCAGCagtccgggcacccccagctctggagcctgacaaggaggacag
gagagat

Embodiment 39: The lentiviral vector according to any one of embodiments 36-38, wherein said ZNF410 shmiRis located upstream or downstream of said anti-sickling (3-globin gene.

Embodiment 40: The lentiviral vector according to any one of embodiments 36-38, wherein said ZNF410 shmiR is disposed in a noncoding sequence of said anti-sickling β-globin gene.

Embodiment 41: The lentiviral vector according to any one of embodiments 14-38, wherein said ZNF410 shmiR is disposed in IVS1 and/or IVS2.

Embodiment 42: The lentiviral vector of embodiment 41, wherein said ZNF410 shmiR is disposed in IVS1.

Embodiment 43: The lentiviral vector of embodiment 42, wherein said ZNF410 shmiRis disposed in the upstream half or quarter of IVS1.

Embodiment 44: The lentiviral vector of embodiment 42, wherein said ZNF410 shmiR is disposed in the downstream half or quarter of IVS1.

Embodiment 45: The lentiviral vector of embodiment 41, wherein said ZNF410 shmiR is disposed in IVS2.

Embodiment 46: The lentiviral vector of embodiment 45, wherein said ZNF410 shmiR is disposed in the upstream half or quarter of IVS2.

Embodiment 47: The lentiviral vector of embodiment 45, wherein said ZNF410 shmiR is disposed in the downstream half or quarter of IVS2.

Embodiment 48: The lentiviral vector of embodiment 45, wherein said ZNF410 shmiR is disposed at or downstream from a deletion in IVS2.

Embodiment 49: The lentiviral vector according to any one of embodiments 36-38, wherein said vector comprises features shown and located as shown in FIG. 4, panel A or panel B.

Embodiment 50: The lentiviral vector of embodiment 1, wherein said vector comprises the nucleic acid sequence shown in Table 9 (SEQ ID NO:17).

Embodiment 51: The lentiviral vector of embodiment 1, wherein said vector comprises the nucleic acid sequence shown in Table 10 (SEQ ID NO:18).

Embodiment 52: The lentiviral vector according to any one of embodiments 1-51, wherein said vector when transduced into a human cell is capable of maintaining % βAS3/VCN expression while increasing fetal globin expression by 10-15 fold.

Embodiment 53: A host cell transduced with a vector according to any one of embodiments 1-52.

Embodiment 54: The host cell of embodiment 53, wherein the cell is a stem cell.

Embodiment 55: The host cell of embodiment 54, wherein said cell is a stem cell derived from bone marrow, and/or from umbilical cord blood, and/or from peripheral blood.

Embodiment 56: The host cell of embodiment 53, wherein the cell is a 293T cell.

Embodiment 57: The host cell of embodiment 53, wherein, wherein the cell is a human hematopoietic progenitor cell.

Embodiment 58: The host cell of embodiment 57, wherein the human hematopoietic progenitor cell is a CD34+ cell.

Embodiment 59: A method of treating a sickle cell disease (SCD), in a subject, said method comprising: transducing a stem cell and/or progenitor cell from said subject with a vector according to any one of embodiments 1-52; and transplanting said transduced cell or cells derived therefrom into said subject where said cells or derivatives therefrom express said anti-sickling human beta globin gene.

Embodiment 60: The method of embodiment 59, wherein the cell is a stem cell.

Embodiment 61: The host cell of embodiment 59, wherein said cell is a stem cell derived from bone marrow.

Embodiment 62: The method of embodiment 59, wherein, wherein the cell is a human hematopoietic progenitor cell.

Embodiment 63: The method of embodiment 62, wherein the human hematopoietic progenitor cell is a CD34+ cell.

It will be noted that while shmiR constructs are preferred in the LV embodiments described herein, in certain embodiments, any one or more of the shmiR constructs described above, can be replaced with a corresponding shRNA construct.

### Definitions

A “reduced length hypersensitive site (HS) sequence” refers to an HS sequence that is shorter in length than the corresponding wild type HS sequence, e.g., HS1, HS2, HS3, and HS4 as previously defined (e.g., HS1, HS2 (˜1.20 kb), HS3 (˜1.28 kb), and HS4 (˜1.1 kb)) (see, e.g., Forrester et al. (1989) Proc. Natl. Acad. Sci. USA, 86: 5439-5443). In certain embodiments the reduced length HS sequence expressly excludes one or more of the HS core sequence(s) as described in PCT Publication No: WO 2013/071309 (PCT/US2012/064878) which is incorporated herein by reference for the core HS sequences described therein (e.g., core HS2 (˜420 bp), core HS3 (˜340 bp), and/or core HS4 (˜410 bp)).

“Recombinant” is used consistently with its usage in the art to refer to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus is one that comprises a recombinant nucleic acid. A recombinant cell is one that comprises a recombinant nucleic acid.

As used herein, the term “recombinant lentiviral vector” or “recombinant LV) refers to an artificially created polynucleotide vector assembled from an LV and a plurality of additional segments as a result of human intervention and manipulation.

By “globin nucleic acid molecule” is meant a nucleic acid molecule that encodes a globin polypeptide. In various embodiments the globin nucleic acid molecule may include regulatory sequences upstream and/or downstream of the coding sequence.

By “globin polypeptide” is meant a protein having at least 85%, or at least 90%, or at least 95%, or at least 98% amino acid sequence identity to a human alpha, beta or gamma globin.

The term “therapeutic functional globin gene” refers to a nucleotide sequence the expression of which leads to a globin that does not produce a hemoglobinopathy phenotype, and which is effective to provide therapeutic benefits to an individual with a defective globin gene. The functional globin gene may encode a wild-type globin appropriate for a mammalian individual to be treated, or it may be a mutant form of globin, preferably one which provides for superior properties, for example superior oxygen transport properties or anti-sickling properties. The functional globin gene includes both exons and introns, as well as globin promoters and splice donors/acceptors.

By “an effective amount” is meant the amount of a required agent or composition comprising the agent to ameliorate or eliminate symptoms of a disease relative to an untreated patient. The effective amount of the composition(s) used to practice the methods described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

As used herein, the terms “shRNA embedded miRNA,” “shmiR” and “shRNA^{miR}” are used interchangeably and refer to an shRNA whose sense and antisense strands are embedded into an miRNA on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins. In some embodiments, a shmiR comprises a 21-nt guide strand, wherein about 17-nt correspond to an antisense RNA that binds a target mRNA and about 4-nt correspond to GC-rich sequences, e.g., GCGC, that improve 3′-end thermodynamic stability in the RNA duplex and promotes preferential RISC loading of the intended guide strand. In one embodiment, the polynucleotide encodes a shmiR.

### BRIEF DESCRIPTION OF THE DRAWINGS

FIG. 1, panels A-B, illustrates a beta-globin lentiviral vector (optionally comprising EC1) (Panel A) and illustrative sites for insertion of an shmiR (panel B). In the illustrated embodiment, a non-sickling gene (PAS3) globin cassette is placed in reverse orientation. In the illustrated embodiment, the beta globin LCR comprises reduced a reduced length hypersensitive site 2 (HS2) sequence, a reduced length hypersensitive site 3 (HS3) sequence, and a reduced length hypersensitive site 4 (HS4) sequence. It will be noted that reduced HS1 (e.g., EC1) is optional. In certain embodiments the vector additionally comprises a minimized LV backbone.

FIG. 2 shows a map of one illustrative lentiviral vector plasmid encompassing a βAS3 gene, containing a reduced HS1 in the LCR and providing an shRNA in intervening sequence 2 (IVS2).

FIG. 3 provides a feature list illustrating features of the vector shown in FIG. 2 and their locations.

FIG. 4, panels A-C illustrates various embodiments of an LV vector comprising a BCL11A shmiR and a ZNF410 shmiR. Panel A shows both the BCL11A shmiR and the ZNF410 shmiR downstream in IVS2, while panel B shows the BCL11A shmiR and the ZNF410 shmiR disposed on each side of a deletion in IVS2. It will be recognized that where BCL11A shmiR and the ZNF410 shmiR are identified in the figure, the positions may be reversed. The two shmiRs need not be confined to IVS2 and one or both can be disposed in IVS1. Panel C illustrates a number of possible positions (A-E in IVS1 and F-J in IVS2) for the BCL11A shmiR and the ZNF410 shmiR, although these positions are illustrative and non-limiting.

FIG. 5 shows that incorporation of shRNA^{miR} (shmiR) into the LV does not severely impact titer.

FIG. 6 illustrates beta-AS3 expression and VCN in cells transfected with the LV comprising a β-AS3 gene and a BCL11A shRNA.

FIG. 7 illustrates total fetal globin expression by cells transfected with the LV comprising a β-AS3 gene and a BCL11A shRNA and expression normalize to VCN.

FIG. 8 illustrates VCN and % anti-sickling globin expression by cells transfected with the LV comprising a β-AS3 gene and a BCL11A shRNA.

FIG. 9 illustrates VCN and % beta-AS3 expression by cells transfected with the LV comprising a β-AS3 gene and a BCL11A shRNA (Experiment 2).

FIG. 10 illustrates % total fetal globin expression and expression normalized to VCN in cells transfected with the LV comprising a 3-AS3 gene and a BCL11A shRNA (Experiment 2).

FIG. 11 illustrates VCN and % anti-sickling globin expression by cells transfected with the LV comprising a 3-AS3 gene and a BCL11A shRNA (round 2).

FIG. 12, top, illustrates the vector copy number (VCN) in health donor cells transduced with a lentiviral vector containing mir223BCL11A-mir144ZNF410 and lacking βAS3, the MiniG-BCL11A-ZNF410-IVS2 del vector containing βAS3 (FIG. 4, panel A (SEQ ID NO:18) (aka miniG B-Z del) and the MiniG-BCL11A-ZNF410-IVS2 end vector containing βAS3 (FIG. 4, panel B, SEQ ID NO: 17) (aka miniG B-Z end). The lower panel shows expression levels of BCL11 and ZNF410 mRNAs.

FIG. 13 shows levels of HBB βAS3 mRNA, and healthy donor gamma hemoglobin mRNA and healthy donor % HbF in the same cells referenced in FIG. 12.

FIG. 14 shows VCN of transduced cells, levels of HBB βAS3 mRNA, and healthy donor gamma hemoglobin mRNA and healthy donor % HbF normalized to vector copy number (VCN).

FIG. 15 shows the decrease in BCL11A mRNA and ZNF410 mRNA normalized to vector copy number.

FIG. 16 top, illustrates the vector copy number (VCN) in cells obtained from a sickle cell disease (SCD) patient transduced with a lentiviral vector containing mir223BCL11A-mir144ZNF410 and lacking βAS3, the MiniG-BCL11A-ZNF410-IVS2 del vector containing βAS3 (FIG. 4, panel A (SEQ ID NO:18) (aka miniG B-Z del) and the MiniG-BCL11A-ZNF410-IVS2 end vector containing βAS3 (FIG. 4, panel B, SEQ ID NO: 17) (aka miniG B-Z end). The lower panel shows expression levels of BCL11 and ZNF410 mRNAs.

FIG. 17 shows levels of SCD patient HBB βAS3 mRNA, SCD patient gamma hemoglobin mRNA, and SCD patient fetal hemoglobin (% HbF) in the same cells referenced in FIG. 16.

FIG. 18 shows levels of HBB βAS3 mRNA in SCD patient cells, SCD patient gamma globin mRNA, and SCD patient % fetal hemoglobin normalized to vector copy number (VCN).

FIG. 19 shows the decrease in BCL11A mRNA and ZNF410 mRNA in SCD patient cells normalized to vector copy number.

FIG. 20 illustrates an analysis of sodium metabisulfite induced sickling in cells from an SCD patient transduced with the BCL11A-ZNF410, MiniG B-Z del, and MiniG B-Z end vectors.

FIG. 21 illustrates an analysis of sodium metabisulfite induced sickling in cells from an SCD patient transduced with the MiniG vector, mir223 BCL11a-mir144ZNF410, MiniG B-Z del, and MiniG B-Z end vectors.

### DETAILED DESCRIPTION

It is believed that autologous stem cell gene therapy for sickle cell disease (SCD) or other hemoglobinopathies (e.g., β-thalassemia, etc.) has the potential to treat these illnesses without the need for immune suppression of current allogeneic hematopoietic stem cell transplantation (HSCT) approaches. In particular it is believed that autologous stem cell gene therapy that introduces, for example, anti-sickling human beta globin into hematopoietic cells (or progenitors thereof) can provide effective therapy for SCD (including, for example, normalized red blood cell (RBC) physiology and prevention of the manifestations of SCD) or certain other hemoglobinopathies.

Current β-globin expression vectors, however, suffer from low vector titer and sub-optimal gene transfer to hematopoietic stem cells, representing a major barrier toward the effective implementation of this gene therapy strategy to the clinic.

It was discovered that one factor significantly impacting viral titer is overall vector length. One solution to reducing vector length has been to reduce LCR size by redefining the boundaries of HS fragments comprising the LCR. However the clinical utility of the reduced length vectors would benefit by increased expression levels.

It was surprising discovered that incorporating an shRNA in a microRNA scaffold, called a shmiR, that inhibits expression of the BCL11A gene (BCL11A shmiR) did not significantly lower titer in comparison to the vector lacking the shRNA construct. Moreover, these vectors were capable of maintaining % βAS3/VCN expression while increasing fetal globin expression by 10-15-fold thereby providing LV vectors that are believed to have significantly improved clinical utility.

Lentiviral Vectors Comprising an Anti-Sickling β-Globin Gene and an shRNA that Improves Globin Expression.

Accordingly, in various embodiments, a recombinant lentiviral vector (LV) is provided where the LV comprises an expression cassette that encodes an anti-sickling β-globin gene where the expression cassette is in reverse orientation in the vector, a β-globin locus control region (LCR) comprising a reduced length hypersensitive site 2 (HS2) sequence, a reduced length hypersensitive site 3 (HS3) sequence, and a reduced length hypersensitive site 4 (HS4) sequence, where the anti-sickling β-globin gene is operably linked to the human β-globin locus control region; and an shRNA that inhibits expression of a BCL11A gene (BCL11AshmiR). In certain embodiments the vector additionally comprises a reduced length hypersensitive site 1 (HS1),

In certain embodiments the vector comprises a modified GLOBE1 (GLOBE-AS3) vector (see, e.g., Poletti et al. (2018) Mol. Ther. Methods Clin. Dev. 11: 167-179) backbone that is modified by the incorporation of an LCR composed o specific HS2 and HS3 sequences (Miccio et al. (2008) Proc. Natl. Acad. Sci. USA, 105(30):10547-10552). In various embodiments, other modifications include, but are not limited to one or more: of the following:
1) A 593 bp deletion beginning for example at position 172 of IVS 2 (e.g., position 5822 in Mini-G End IVS2 shmiR Lentiviral Vector (SEQ ID NO:12);
2) Changes in the CPPT and surrounding area such as:
   a) change of GTTAAC to ATCTCGAcacaaatggcagtattcatccacaa (SEQ ID NO:1) (e.g., shown as bp 4924 to 4931 in mini-G end IVS2 shmiR lentiviral vector (SEQ ID NO:12);
   b) an additional “A” (e.g., shown at position 5062 in mini-G end IVS2 shmiR lentiviral vector (SEQ ID NO:12);
   c) change of ATCGGTACGTAC (SEQ ID NO:2) to CGGGTTTATTACAGGGACAGCAGA (SEQ ID NO:3) (e.g., shown as bp 5074 to 5097 in mini-G end IVS2 shmiR lentiviral vector (SEQ ID NO:12).

It is noted that in certain embodiments, the Mini-G vector provides the backbone for the LV constructs described herein. In the nomenclature of Morgan et al. (2020) Mol. Ther. 28(1): 328-340, this vector is also known as UV+EC1 because it also has reduced length HS site 1 component added to the UV vector. This was the vector utilized in Example 1. It will be recognized, however, that in certain embodiments the HS1 site can be eliminated.

These modifications of the vector are illustrative, but non-limiting. It is noted that in certain embodiments, a variety of reduced length HS1, HS2, HS3, and HS4 sequences are known (see, e.g., PCT Publication No: WO 2020/168004 (PCT/US2020/017998); Morgan et al. (2019) Mol. Ther., 28(1): 328-340; and the like) and their incorporation in various LV vectors described herein is provided.

As noted above, in various embodiments, the vector comprises reduced length HS1, HS2, HS3, and HS4. In certain embodiments the reduced length hypersensitive site 1 (HS1) sequence consists of the nucleotide sequence of EC1 shown in Table 1 (SEQ ID NO:7). In certain embodiments the reduced length hypersensitive site 2 (HS2) sequence consists of the nucleotide sequence of EC2 shown in Table 1 (SEQ ID NO:4). In certain embodiments the reduced length hypersensitive site 3 (HS3) sequence consists of the nucleotide sequence of EC3 shown in Table 1 (SEQ ID NO:5). In certain embodiments the reduced length hypersensitive site 4 (HS4) sequence consists of the nucleotide sequence of EC4 shown in Table 1 (SEQ ID NO: 6).

TABLE 1
Nucleic acid sequences of illustrative reduced
length hypersensitive site.

| | |
|---|---|
| HS2 | TACGTaTATGTGTATATATATATATATATTCAGGAAATAATAT |
| (aka ecCORE | ATTCTAGAATATGTCACATTCTGTCTCAGGCATCCATTTTCTT |
| HS2 or EC2) | TATGATGCCGTTTGAGGTGGAGTTTTAGTCAGGTGGTCAGCT |
| (SEQ ID NO: 4) | TCTCCTTTTTTTTGCCATCTGCCCTGTAAGCATCCTGCTGGGG |
| | ACCCAGATAGGAGTCATCACTCTAGGCTGAGAACATCTGGG |
| | CACACACCCTAAGCCTCAGCATGACTCATCATGACTCAGCAT |
| | TGCTGTGCTTGAGCCAGAAGGTTTGCTTAGAAGGTTACACAG |
| | AACCAGAAGGCGGGGGTGGGGCACTGACCCCGACAGGGGC |
| | CTGGCCAGAACTGCTCATGCTTGGACTATGGGAGGTCACTAA |
| | TGGAGACACACAGAAATGTAACAGGAACTAAGGAAAAACT |
| | GAAGCTT |
| HS3 | TGGGGGTATAGGGGAGCAGTCCCATGTAGTAGTAGAATGAA |
| (aka ecCORE HS3 | AAATGCTGCTATGCTGTGCCTCCCCCACCTTTCCCATGTCTGC |
| or EC3) | CCTCTACTCATGGTCTATCTCTCCTGGCTCCTGGGAGTCATG |
| (SEQ ID NO: 5) | GACTCCACCCAGCACCACCAACCTGACCTAACCACCTATCTG |
| | AGCCTGCCAGCCTATAACCCATCTGGGCCCTGATAGCTGGTG |
| | GCCAGCCCTGACCCCACCCCACCCTCCCTGGAACCTCTGATA |
| | GACACATCTGGCACACCAGCTCGCAAAGTCACCGTGAGGGT |
| | CTTGTGTTTGCTGAGTCAAAATTCCTTGAAATCCAAGTCCTT |
| | AGAGACTCC |
| HS4 | CAGGCTTGGATTCAAAGCTCCTGACTTTCTGTCTAGTGTATG |
| (aka ecCORE HS4 | TGCAGTGAGCCCCTTTTCCTCTAACTGAAAGAAGGAAAAAA |
| or EC4) | AAATGGAACCCAAAATATTCTACATAGTTTCCATGTCACAGC |
| (SEQ ID NO: 6) | CAGGGCTGGGCAGTCTCCTGTTATTTCTTTTAAAATAAATAT |
| | ATCATTTAAATGCATAAATAAGCAAACCCTGCTCGGGAATG |
| | GGAGGGAGAGTCTCTGGAGTCCACCCCTTCTCGGCCCTGGCT |
| | CTGCAGATAGTGCTATCAAAGCCCTGACAGAGCCCTGCCCAT |
| | TGCTGGGCCTTGGAGTGAGTCAGCCTAGTAGAGAGGCAGGG |
| | CAAGCCATCTCATAGCTGCTGAGTGGGAGAGAGAAAAGGGC |
| | TCATTGTCTATAAACTCAGGTCATGGCTATTCTTAT |
| HS1 | CATCAATAATTCTAGCCCCACAGGAGTTTGTTCTGAAAGTAA |
| (aka ecCORE HS1 | ACTTCCACAACCGCAAGCTTATTGAGGCTAAGGCATCTGTGA |
| or EC1) | AGGAAAGAAACATCTCCTCTAAACCACTATGCTGCTAGAGC |
| (SEQ ID NO: 7) | CTCTTTTCTGTACTCAAGCCTCATTCAGACACTAGTGTCACC |
| | AGTCTCCTCATATACCTATTGTATTTTCTTCTTCTTGCTGGTTT |
| | AGTCATGTTTTCTGGGAGCTTAGGGGCTTATTTTATTTTGTTT |
| | TGTTTTCTAATCAACAGAGATGGGCAAACCCATTATTTTTTT |
| | CTTTAGACTTGGGATGGTGATAGCTGGGCAGCGTCAGAAAC |
| | TGTGTGTGGATATAGATAAGAGCTCAGGACTATGCTGAGCT |
| | GTGATGAGGGAGGGGCCTAGCTAAAGGCAGTGAGAGTCAGA |
| | ATGCTCCTGCTATTGCCTTCTCAGTCCCCACGCTTGGTTTCTA |
| | CACAAGTAGATACATAGAAAAGGCTATAGGTTAGTGTTTGA |
| | GAGTCCTGCATGATTAGTTGCTCAGAAATGCCCGATAAATAT |
| | GTTATGTGTGTTTATGTATATATATGTTTTATATGTGTGTGTG |
| | TGTGTGTTGTGTTTACAAATATGTGATTATCATCAAAACGTG |
| | AGGG |

While not required, in certain embodiments, the lentiviral vector additionally comprises a hypersensitive site 1 or a reduced length hypersensitive site 1 (HS1). In certain embodiments the reduced length hypersensitive site 1 (HS1) sequence consists of the nucleotide sequence of EC1 shown in Table 1 (SEQ ID NO:7).

As noted above, in various embodiments, the recombinant lentiviral vectors described herein comprise an shRNA that inhibits expression of a BCL11A gene (BCL11A shRNA). It has been observed that BCL11A has been identified as a regulator involved in the fetal-to-adult hemoglobin switch in humans. Additionally, BCL11A appears to be an essential transcription factor required for B lymphocyte development. Downregulation of BCL11A expression by small hairpin RNAs (shRNAs) expressed by polymerase (pol) III promoters in lentivirus vectors has been observed to lead to rapid and sustained reactivation of γ-globin expression and induction of HbF (α₂γ₂) expression in adult erythroid precursor cells.

BCL11A shRNA sequence are known to those of skill in the art, and can be found, for example, in Brendel et al. (2016) J. Clin. Invest. 126(10): 3868-3878 and in certain embodiments, the use of any of the BCL11A shRNAs described therein (as well as other BCL11A shRNAs) is contemplated.

It has been observed that high-level expression of shRNAs in mammalian cells typically using pol III promoters can be associated with nonspecific cellular toxicities previously identified highly effective shRNAs targeting the BCL11A mRNA.

However it was observed that engineering the shRNAS so that they are embedded within an miRNA (ShRNA^{miR}) can achieve ubiquitous knockdown of BCL11A and significantly reduce nonspecific cellular toxicities. In particular, it was shown that by utilizing lineage-specific and miRNA-embedded expression of BCL11A-targeting shRNAs, lentivirus vectors can effect γ-globin induction leading to clinically significant increases in RBC HbF while obviating toxicity (see, e.g., Brendel et al. (2016) J. Clin. Invest. 126(10): 3868-3878).

Accordingly, in certain embodiments the shRNAs in the lentiviral vectors described herein are provided in microRNA constructs. In one illustrative, but non-limiting embodiment, BCL11A shRNA is embedded in a microRNA 223 (MIR223). In certain embodiments the BCL11A shmiR in miR223 comprises or consists of the sequence shown in Table 2 (SEQ ID NO:8). It will be recognized that the BCL11A shRNA sequence and the microRNA shown therein are illustrative and non-limiting and using the teaching provided herein, other BCL11A shRNA embedded in microRNAs will be available to one of skill in the art.

TABLE 2
Illustrative shRNA constructs.

| shRNA | | SEQ |
|---|---|---|
| construct | NT Sequence | ID NO |
| BCL11A shmiR | GATCTCACTTCCCCACAGAAGCTCTTGGCCTGGCCTC | 8 |
| in miR223 | CTGCAGTGCCACGCTGCGCGATCGAGTGTTGAATAA | |
| Fwd | ctccatgtggtagagTTATTCAACACTCGATCGCGCAGTGCG | |
| orientation | GCACATGCTTACCAGCTCTAGGCCAGGGCAGATGGG | |
| | ATATGACGAATGGACTGCCAGCTGGATACAAGGATG | |
| | CTCACC | |
| BCL11A shmiR | GGTGAGCATCCTTGTATCCAGCTGGCAGTCCATTCGT | 9 |
| in miR223 | CATATCCCATCTGCCCTGGCCTAGAGCTGGTAAGCA | |
| Rev | TGTGCCGCACTGCGCGATCGAGTGTTGAATAActctacc | |
| complement | acatggagTTATTCAACACTCGATCGCGCAGCGTGGCAC | |
| | TGCAGGAGGCCAGGCCAAGAGCTTCTGTGGGGAAGT | |
| | GAGATC | |
| ZNF410 shmiR | cgcttttcaagccatgcttcctgtgcccccagtggggccctggct | 10 |
| in miR 144 | gGCTGAGCACTTAGTGTTTGTAagtttgcgatgagacacTACAAA | |
| Fwd. | CACTAAGTGCTCAGCagtccgggcacccccagctctggagcctga | |
| Orientation | caaggaggacaggagagat | |
| ZNF410 shmiR | ATCTCTCCTGTCCTCCTTGTCAGGCTCCAGAGCTGGG | 11 |
| in miR 144 | GGTGCCCGGACTGCTGAGCACTTAGTGTTTGTAGTG | |
| Rev | TCTCATCGCAAACTTACAAACACTAAGTGCTCAGCC | |
| complement | AGCCAGGGCCCCACTGGGGGCACAGGAAGCATGGC | |
| | TTGAAAAGCG | |

In certain embodiments the BLC11A shRNA construct can be disposed in the vector upstream or downstream of the anti-sickling gene. In certain embodiments the BCL11A shRNA can be embedded in a non-coding sequence in the anti-sickling gene.

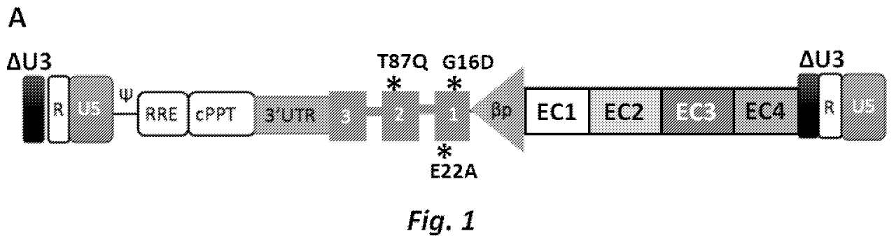

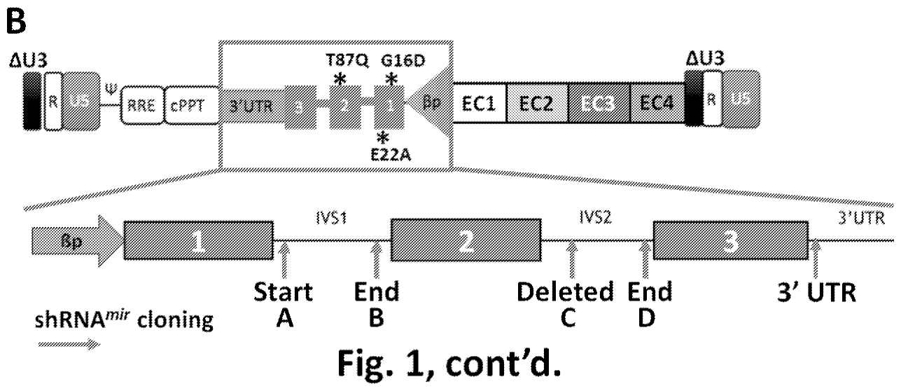

In various embodiments the beta-globin gene comprises two intervening sequences (IVS1 and IVS2) that divide it into three discontinuous segments. In certain embodiments the BCL11A shRNA construct is disposed in IVS1 while in other embodiments the BCL11A shRNA construct is disposed in IVS2. Where there are two or more BCL11A shRNA constructs in the LV, one construct can be disposed in IVS1 and the other construct in IVS2, or two or more constructs can be disposed in IVS1 and/or IVS2. In certain embodiments the BCL11A shRNA construct is disposed in the upstream ½ or upstream ¼ of IVS1 (see, e.g., position A in FIG. 1, panel B) or in the downstream ½ or ¼ of IVS1 (see, e.g., position B in FIG. 1, panel B). In certain embodiments the BCL11A shRNA construct is disposed in the upstream ½ or upstream ¼ of IVS2 (see, e.g., position C in FIG. 1, panel B) or in the downstream ½ or ¼ of IVS2 (see, e.g., position D in FIG. 1, panel B). Where IVS2 comprises a deletion, the BCL11A shRNA construct can be disposed upstream of the deletion or downstream of the deletion or located at the deletion.

In certain embodiments, a lentivirus vector provided herein comprises the nucleic acid sequence shown in Table 4 (SEQ ID NO:12). In certain embodiments, a lentivirus vector provided herein comprises the nucleic acid sequence shown in Table 5 (SEQ ID NO:13). In certain embodiments, a lentivirus vector provided herein comprises the nucleic acid sequence shown in Table 6 (SEQ ID NO:14). In certain embodiments, a lentivirus vector provided herein comprises the nucleic acid sequence shown in Table 7 (SEQ ID NO: 15).

The gene ZNF410 (encoding zinc finger protein (ZNF) 410) has been identified as a fetal hemoglobin (HbF) repressor. ZNF410 does not bind directly to the genes encoding γ-globins, but rather its chromatin occupancy is concentrated solely at CHD4, encoding the NuRD nucleosome remodeler, which is itself required for HbF repression. CHD4 has two ZNF410-bound regulatory elements with 27 combined ZNF410 binding motifs constituting unparalleled genomic clusters. These elements completely account for the effects of ZNF410 on fetal globin repression. Knockout of ZNF410 or its mouse homolog Zfp410 reduces CHD4 levels by 60%, enough to substantially de-repress HbF while eluding cellular or organismal toxicity (see, e.g., Vinjamur et al. (2021) Nat. Genetics, 53: 719-728).

Accordingly, in certain embodiments, to further improve fetal hemoglobin expression, the LV vectors described herein can additionally include an shRNA that inhibits expression of ZNF410. In various embodiments the ZNF410 shRNA is provided as ZNF410 shRNA engineered into a micro-RNA scaffold to provide ZNF410 shmiR. Numerous ZNF410 shRNA sequence are known to those of skill in the art, and AAV vectors that silence ZNF410 using ZNF410 shRNA are commercially available (see, e.g., Vector Biolabs™ AAV-h-ZNF410-shRNA). Using the teachings provided herein, ZNF410 shmiRs incorporating these shRNAs will be readily available to one of skill in the art.

In certain embodiments the ZNF410 shRNA is embedded in microRNA 144 (MIR144). In certain embodiments the ZNF410 shmiR in miR144 comprises or consists of the sequence shown in Table 2 (SEQ ID NO:10). It will be recognized that the ZNF410 shRNA sequence and the microRNA shown therein are illustrative and non-limiting and using the teaching provided herein, other ZNF410 shRNA embedded in microRNAs will be available to one of skill in the art.

Where the LV described herein contains both a BCL11A shRNA (e.g., as a BCL11A shmiR) and a ZNF410 shRNA (e.g., as a ZNF410 shmiR), one or both constructs can be disposed in the vector upstream or downstream of the anti-sickling gene or one construct can be upstream and the other construct downstream of the anti-sickling gene. In certain embodiments one or both constructs can be embedded in a non-coding sequence in the anti-sickling gene.

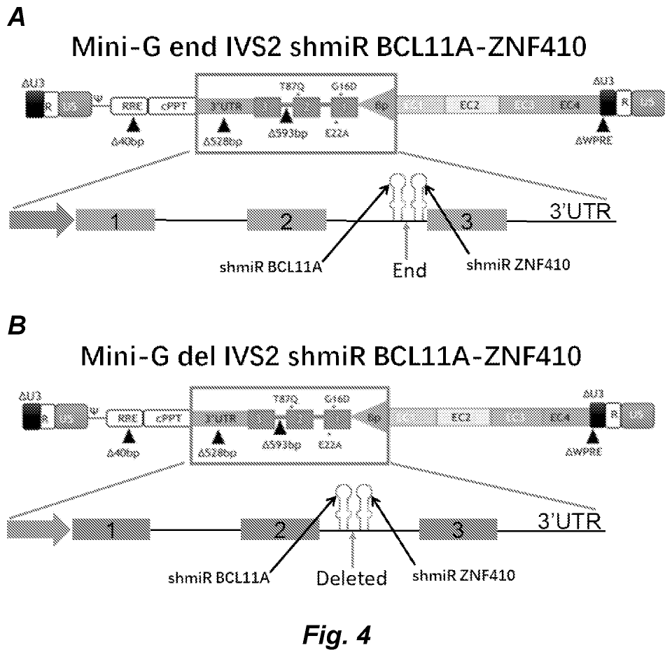

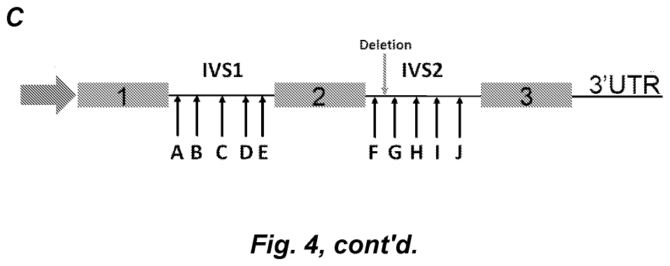

In certain illustrative, but non-limiting, embodiments one or both constructs can be disposed in an intervening sequence, e.g., IVS1 and/or IVS2. FIG. 4, panels A-C illustrates various embodiments of an LV vector comprising a BCL11A shRNA and a ZNF410 shRNA. Panel A shows both the BCL11A shmiR and the ZNF410 shmiR downstream in IVS2, while panel B shows the BCL11A shmiR and the ANF410 shmiR disposed on each side of a deletion in IVS2. It will be recognized that where BCL11A shmiR and the ZNF410 shmiR are identified in the figure, the positions may be reversed. The two shmiRs need not be confined to IVS2 and one or both can be disposed in IVS1.

FIG. 4, panel C, illustrates a number of possible positions (A-E in IVS1 and F-J in IVS2) for the BCL11A shmiR and the ZNF410 shmiR, although these positions are illustrative and non-limiting. It will be recognized that in various embodiments any combination of these positions can be utilized. Illustrative, but non-limiting combinations of these positions for the BCL11A shmiR and the ZNF410 shmiR are shown in Table 3.

TABLE 3
Illustrative combinations of positions (shown in FIG.
4, panel C) for the BCL11A shmiR and the ZNF410 shmiR.

| | | BCL11A shRNA Location | | | | | | | | |
|---|---|---|---|---|---|---|---|---|---|---|
| | Location | A | B | C | D | E | F | G | H | I |
| ZNF410 | A | | X | X | X | X | X | X | X | X |
| shRNA | B | X | | X | X | X | X | X | X | X |
| location | C | X | X | | X | X | X | X | X | X |
| | D | X | X | X | | X | X | X | X | X |
| | E | X | X | X | X | | X | X | X | X |
| | F | X | X | X | X | X | | X | X | X |
| | G | X | X | X | X | X | X | | X | X |
| | H | X | X | X | X | X | X | X | | X |
| | I | X | X | X | X | X | X | X | X | |

In certain embodiments, a lentivirus vector provided herein comprises the comprises features shown and located as shown in FIG. 4, panel A or panel B. n certain embodiments, a lentivirus vector provided herein comprises the nucleic acid sequence shown in Table 9 (SEQ ID NO:17). In certain embodiments, a lentivirus vector provided herein comprises the nucleic acid sequence shown in Table 10 (SEQ ID NO:18).

The foregoing shRNAs and shRNA construct(s) and locations thereof in the lentivirus are illustrative and non-limiting. Using the teaching provided herein, numerous other BCL11A shmiRs, ZNF410 shmiRs, and locations thereof will be available to one of skill in the art.

As explained above, in various embodiments, the recombinant lentiviral vectors described herein comprise an anti-sickling human beta globin gene encoding an anti-sickling-beta globin polypeptide. One illustrative, but non-limiting cassette is βAS3 which comprises an ˜2.3 kb recombinant human β-globin gene (exons and introns) with three amino acid substitutions (Thr87Gln; Gly16Asp; and Glu22Ala) under the control of transcriptional control elements (e.g., the human β-globin gene 5′ promoter (e.g., −266 bp), the human β-globin 3′ enhancer (e.g., ˜260 bp), β-globin intron 2 with a ˜375 bp RsaI deletion from IVS2, and a ˜3.4 kb composite human S-globin locus control region (e.g., HS2˜1203 bp; HS3˜1213 bp; HS4˜954 bp). One embodiment of a βAS3 cassette is described by Levasseur (2003) Blood 102: 4312-4319.

In certain embodiments the β-globin gene comprises a SspI (S) to RsaI (R) deletion (˜220 bp), e.g., as described by Antoniou et al. 1998) Nucl. Acids Res., 26(3): 721-729. In certain embodiments the beta-globin gene comprise an IVS2 deletion of about 993 bp (e.g., as shown in the illustrative vectors in Tables 4-7).

The βAS3 cassette, however, is illustrative and need not be limiting. Using the teaching provided herein, numerous variations will be available to one of skill in the art. Such variations include, for example, use of a gene encoding a wild-type β-globin, use of a gene comprising one or two mutations selected from the group consisting of Thr87Gln, Gly16Asp, and Glu22Ala, and/or further or alternative mutations to the β-globin to further enhance non-sickling properties, alterations in the transcriptional control elements (e.g., promoter and/or enhancer), variations on the intron size/structure, and the like.

In view of the foregoing, an improved LV is provided for the introduction of a normal wild-type or an anti-sickling beta globin into stem and progenitor cells (e.g., hematopoietic stem and progenitor cells) that can then be transplanted into a subject in need thereof (e.g., a subject that has the sickle cell mutation, a subject with β-thalassemia, etc.).

In various embodiments the improved vectors described herein are capable of driving lineage-restricted expression of an anti-sickling β-globin like gene (βAS3), a wild-type β-globin gene, or any other heterologous gene it is desired to express. Optimization of the LCR, as described above, with the primary goal of reducing length provides smaller effective vectors with improved enhancer activity. Moreover, incorporation of the shRNAs as described herein can improve the expression of fetal hemoglobin (fHB) thereby improving the therapeutic efficacy of the vector.

Additionally, in certain embodiments, elements can be added to the optimized vectors such as the murine GATA1. In certain embodiments a human Ankyrin insulator (˜150 bp) element can be included. These vectors, rationally designed for reduced sizes of the LCR fragments and added transcriptional enhancing elements are believed to be produced at higher titers than the original β-globin lentiviral vector and have improved gene transfer to human HSC while retaining strong erythroid-specific gene expression. Such improved lentiviral vectors can be effective for gene therapy of sickle cell disease.

In certain embodiments, one or more of the reduced HS sequences described herein can be used in combination with a full-length (e.g., wildtype) HS sequence.

In various embodiments, the LVs described herein can, optionally, have additional safety features that can include, for example, the presence of an insulator (e.g., an FB insulator in the 3′LTR). Additionally, or alternatively, in certain embodiments, the HIV LTR has been substituted with an alternative promoter (e.g., a CMV) to yield a higher titer vector without the inclusion of the HIV TAT protein during packaging. Other strong promoters (e.g., RSV, and the like can also be used).

In view of the results provided in the accompanying examples, it is believed that LVs described herein, e.g., recombinant TAT-independent, SIN LVs that express a human beta-globin gene and a BCL11A shRNA and/or a ZNF410 shRNA can be used to effectively treat sickle cell disease (SCD) in a subject (e.g., human and non-human mammals).

It is believed these vectors can be used for the modification of stem cells (e.g., hematopoietic stem and progenitor cells) that can be introduced into a subject in need thereof for the treatment of, e.g., SCD. Moreover, it appears that the resulting cells will produce enough of the transgenic β-globin and fetal β-globin protein to demonstrate significant improvement in subject health. It is also believed the vectors can be directly administered to a subject to achieve in vivo transduction of the target (e.g., hematopoietic stem or progenitor cells) and thereby also effect a treatment of subjects in need thereof.

### TAT-Independent and Self Inactivating Lentiviral Vectors.

As noted above, in various embodiments the LVs described herein can comprise various safety features. For example, the HIV LTR has been substituted with a CMV promoter to yield higher titer vector without the inclusion of the HIV TAT protein during packaging. In certain embodiments an insulator (e.g., the FB insulator) is introduced into the 3′LTR for safety. The LVs are also constructed to provide efficient transduction and high titer.

To further improve safety, in various embodiments, the lentiviral vectors described herein comprise a TAT-independent, self-inactivating (SIN) configuration. Thus, in various embodiments it is desirable to employ in the LVs described herein an LTR region that has reduced promoter activity relative to wild-type LTR. Such constructs can be provided that are effectively “self-inactivating” (SIN) which provides a biosafety feature. SIN vectors are ones in which the production of full-length vector RNA in transduced cells is greatly reduced or abolished altogether. This feature minimizes the risk that replication-competent recombinants (RCRs) will emerge. Furthermore, it reduces the risk that that cellular coding sequences located adjacent to the vector integration site will be aberrantly expressed.

Furthermore, a SIN design reduces the possibility of interference between the LTR and the promoter that is driving the expression of the transgene. SIN LVs can often permit full activity of the internal promoter.

The SIN design increases the biosafety of the LVs. The majority of the HIV LTR is comprised of the U3 sequences. The U3 region contains the enhancer and promoter elements that modulate basal and induced expression of the HIV genome in infected cells and in response to cell activation. Several of these promoter elements are essential for viral replication. Some of the enhancer elements are highly conserved among viral isolates and have been implicated as critical virulence factors in viral pathogenesis. The enhancer elements may act to influence replication rates in the different cellular target of the virus

As viral transcription starts at the 3′ end of the U3 region of the 5′ LTR, those sequences are not part of the viral mRNA and a copy thereof from the 3′ LTR acts as template for the generation of both LTR's in the integrated provirus. If the 3′ copy of the U3 region is altered in a retroviral vector construct, the vector RNA is still produced from the intact 5′ LTR in producer cells but cannot be regenerated in target cells. Transduction of such a vector results in the inactivation of both LTR's in the progeny virus. Thus, the retrovirus is self-inactivating (SIN) and those vectors are known as SIN transfer vectors.

In certain embodiments self-inactivation is achieved through the introduction of a deletion in the U3 region of the 3′ LTR of the vector DNA, i.e., the DNA used to produce the vector RNA. During RT, this deletion is transferred to the 5′ LTR of the proviral DNA. Typically, it is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function typically spread out over U3, R and U5. Accordingly, in certain embodiments, it is desirable to eliminate as many of the transcriptionally important motifs from the LTR as possible while sparing the polyadenylation determinants.

The SIN design is described in detail in Zufferey et al. (1998) J Virol. 72(12): 9873-9880, and in U.S. Pat. No. 5,994,136. As described therein, there are, however, limits to the extent of the deletion at the 3′ LTR. First, the 5′ end of the U3 region serves another essential function in vector transfer, being required for integration (terminal dinucleotide+att sequence). Thus, the terminal dinucleotide and the att sequence may represent the 5′ boundary of the U3 sequences which can be deleted. In addition, some loosely defined regions may influence the activity of the downstream polyadenylation site in the R region. Excessive deletion of U3 sequence from the 3′LTR may decrease polyadenylation of vector transcripts with adverse consequences both on the titer of the vector in producer cells and the transgene expression in target cells.

Additional SIN designs are described in U.S. Patent Publication No: 2003/0039636. As described therein, in certain embodiments, the lentiviral sequences removed from the LTRs are replaced with comparable sequences from a non-lentiviral retrovirus, thereby forming hybrid LTRs. In particular, the lentiviral R region within the LTR can be replaced in whole or in part by the R region from a non-lentiviral retrovirus. In certain embodiments, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions.

Accordingly, in certain embodiments, the SIN configuration provides a retroviral LTR comprising a hybrid lentiviral R region that lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region. In a particular embodiment, the retroviral LTR comprises a hybrid R region, wherein the hybrid R region comprises a portion of the HIV R region (e.g., a portion comprising or consisting of the nucleotide sequence shown in SEQ ID NO: 10 in US 2003/0039636) lacking the TAR sequence, and a portion of the MoMSV R region (e.g., a portion comprising or consisting of the nucleotide sequence shown in SEQ ID NO: 9 in 2003/0039636) comparable to the TAR sequence lacking from the HIV R region. In another particular embodiment, the entire hybrid R region comprises or consists of the nucleotide sequence shown in SEQ ID NO: 11 in 2003/0039636.

Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMLV, Friend, MSCV, RSV and Spumaviruses. In one illustrative embodiment, the lentivirus is HIV and the non-lentiviral retrovirus is MoMSV.

In another embodiment described in US 2003/0039636, the LTR comprising a hybrid R region is a left (5′) LTR and further comprises a promoter sequence upstream from the hybrid R region. Preferred promoters are non-lentiviral in origin and include, for example, the U3 region from a non-lentiviral retrovirus (e.g., the MoMSV U3 region). In one particular embodiment, the U3 region comprises the nucleotide sequence shown in SEQ ID NO: 12 in US 2003/0039636. In another embodiment, the left (5′) LTR further comprises a lentiviral U5 region downstream from the hybrid R region. In one embodiment, the U5 region is the HIV U5 region including the HIV att site necessary for genomic integration. In another embodiment, the U5 region comprises the nucleotide sequence shown in SEQ ID NO: 13 in US 2003/0039636. In yet another embodiment, the entire left (5′) hybrid LTR comprises the nucleotide sequence shown in SEQ ID NO: 1 in US 2003/0039636.

In another illustrative embodiment, the LTR comprising a hybrid R region is a right (3′) LTR and further comprises a modified (e.g., truncated) lentiviral U3 region upstream from the hybrid R region. The modified lentiviral U3 region can include the att sequence, but lack any sequences having promoter activity, thereby causing the vector to be SIN in that viral transcription cannot go beyond the first round of replication following chromosomal integration. In a particular embodiment, the modified lentiviral U3 region upstream from the hybrid R region consists of the 3′ end of a lentiviral (e.g., HIV) U3 region up to and including the lentiviral U3 att site. In one embodiment, the U3 region comprises the nucleotide sequence shown in SEQ ID NO: 15 in US 2003/0039636. In another embodiment, the right (3′) LTR further comprises a polyadenylation sequence downstream from the hybrid R region. In another embodiment, the polyadenylation sequence comprises the nucleotide sequence shown in SEQ ID NO: 16 in US 2003/0039636. In yet another embodiment, the entire right (5′) LTR comprises the nucleotide sequence shown in SEQ ID NO: 2 or 17 of US 2003/0039636.

Thus, in the case of HIV based LV, it has been discovered that such vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower.

It has also been demonstrated that the trans-acting function of Tat becomes dispensable if part of the upstream LTR in the transfer vector construct is replaced by constitutively active promoter sequences (see, e.g., Dull et al. (1998) J Virol. 72(11): 8463-8471. Furthermore, we show that the expression of rev in trans allows the production of high-titer HIV-derived vector stocks from a packaging construct which contains only gag and pol. This design makes the expression of the packaging functions conditional on complementation available only in producer cells. The resulting gene delivery system, conserves only three of the nine genes of HIV-1 and relies on four separate transcriptional units for the production of transducing particles.

In one embodiments illustrated in Example 1, the cassette expressing an anti-sickling β-globin (e.g., βAS3) is placed in the pCCL LV backbone, which is a SIN vector with the CMV enhancer/promoter substituted in the 5′ LTR.

It will be recognized that the CMV promoter typically provides a high level of non-tissue specific expression. Other promoters with similar constitutive activity include, but are not limited to the RSV promoter, and the SV40 promoter. Mammalian promoters such as the beta-actin promoter, ubiquitin C promoter, elongation factor 1αpromoter, tubulin promoter, etc., may also be used.

The foregoing SIN configurations are illustrative and non-limiting. Numerous SIN configurations are known to those of skill in the art. As indicated above, in certain embodiments, the LTR transcription is reduced by about 95% to about 99%. In certain embodiments LTR may be rendered at least about 90%, at least about 91%, at least about 92%, at least about 93%, at least about 94%, at least about 95% at least about 96%, at least about 97%, at least about 98%, or at least about 99% transcriptionally inactive.

### Insulator Element

In certain embodiments, to further enhance biosafety, insulators are inserted into the lentiviral vectors described herein. Insulators are DNA sequence elements present throughout the genome. They bind proteins that modify chromatin and alter regional gene expression. The placement of insulators in the vectors described herein offer various potential benefits including, inter alia: 1) Shielding of the vector from positional effect variegation of expression by flanking chromosomes (i.e., barrier activity); and 2) Shielding flanking chromosomes from insertional trans-activation of gene expression by the vector (enhancer blocking). Thus, insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Natl. Acad. Sci. USA, 99: 16433; and Zhan et al. (2001) Hum. Genet., 109: 471). In the present context insulators may contribute to protecting lentivirus-expressed sequences from integration site effects, which may be mediated by cis-acting elements present in genomic DNA and lead to deregulated expression of transferred sequences. In various embodiments LVs are provided in which an insulator sequence is inserted into one or both LTRs or elsewhere in the region of the vector that integrates into the cellular genome.

The first and best characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau et al. (1999) EMBO J. 18: 4035-4048). A 1.2-kb fragment containing the cHS4 element displays classic insulator activities, including the ability to block the interaction of globin gene promoters and enhancers in cell lines (Chung et al. (1993) Cell, 74: 505-514), and the ability to protect expression cassettes in Drosophila (Id.), transformed cell lines (Pikaart et al. (1998) Genes Dev. 12: 2852-2862), and transgenic mammals (Wang et al. (1997) Nat. Biotechnol., 15: 239-243; Taboit-Dameron et al. (1999) Transgenic Res., 8: 223-235) from position effects. Much of this activity is contained in a 250-bp fragment. Within this stretch is a 49-bp cHS4 core (Chung et al. (1997) Proc. Natl. Acad. Sci., USA, 94: 575-580) that interacts with the zinc finger DNA binding protein CTCF implicated in enhancer-blocking assays (Bell et al. (1999) Cell, 98: 387-396).

One illustrative and suitable insulator is FB (FII/BEAD-A), a 77 bp insulator element, that contains the minimal CTCF binding site enhancer-blocking components of the chicken β-globin 5′ HS4 insulators and a homologous region from the human T-cell receptor alpha/delta blocking element alpha/delta I (BEAD-I) insulator described by Ramezani et al. (2008) Stem Cell 26: 3257-3266. The FB “synthetic” insulator has full enhancer blocking activity. This insulator is illustrative and non-limiting. Other suitable insulators may be used including, for example, the full-length chicken beta-globin HS4 or insulator sub-fragments thereof, the ankyrin gene insulator, and other synthetic insulator elements.

### Packaging Signal

In various embodiments the vectors described herein further comprise a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

### Rev Responsive Element (RRE).

In certain embodiments the lentiviral vectors described herein comprise a Rev response element (RRE) to enhance nuclear export of unspliced RNA. RREs are well known to those of skill in the art. Illustrative RREs include, but are not limited to RREs such as that located at positions 7622-8459 in the HIV NL4-3 genome (Genbank accession number AF003887) as well as RREs from other strains of HIV or other retroviruses. Such sequences are readily available from Genbank or from the database with URL hiv-web.lanl.gov/content/index.

Central PolyPurine Tract (cPPT).

In various embodiments the lentiviral vectors described herein further include a central polypurine tract. Insertion of a fragment containing the central polypurine tract (cPPT) in lentiviral (e.g., HIV-1) vector constructs is known to enhance transduction efficiency drastically, reportedly by facilitating the nuclear import of viral cDNA through a central DNA flap.

### Expression-Stimulating Posttranscriptional Regulatory Element (PRE)

In certain embodiments the lentiviral vectors (LVs) described herein may comprise any of a variety of posttranscriptional regulatory elements (PREs) whose presence within a transcript increases expression of the heterologous nucleic acid (e.g., βAS3) at the protein level. PREs may be particularly useful in certain embodiments, especially those that involve lentiviral constructs with modest promoters.

One type of PRE is an intron positioned within the expression cassette, which can stimulate gene expression. However, introns can be spliced out during the life cycle events of a lentivirus. Hence, if introns are used as PRE's they are typically placed in an opposite orientation to the vector genomic transcript.

Posttranscriptional regulatory elements that do not rely on splicing events offer the advantage of not being removed during the viral life cycle. Some examples are the posttranscriptional processing element of herpes simplex virus, the posttranscriptional regulatory element of the hepatitis B virus (HPRE) and the woodchuck hepatitis virus (WPRE). Of these the WPRE is typically preferred as it contains an additional cis-acting element not found in the HPRE. This regulatory element is typically positioned within the vector so as to be included in the RNA transcript of the transgene, but outside of stop codon of the transgene translational unit.

The WPRE is characterized and described in U.S. Pat. No. 6,136,597. As described therein, the WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. Presence of the WPRE in the sense orientation was shown to increase transgene expression by up to 7- to 10-fold. Retroviral vectors transfer sequences in the form of cDNAs instead of complete intron-containing genes as introns are generally spliced out during the sequence of events leading to the formation of the retroviral particle. Introns mediate the interaction of primary transcripts with the splicing machinery. Because the processing of RNAs by the splicing machinery facilitates their cytoplasmic export, due to a coupling between the splicing and transport machineries, cDNAs are often inefficiently expressed. Thus, the inclusion of the WPRE in a vector results in enhanced expression of transgenes.

### Transduced Host Cells and Methods of Cell Transduction.

The recombinant lentiviral vectors (LV) and resulting virus described herein are capable of transferring a heterologous nucleic acid (e.g., a nucleic acid encoding an anti-sickling β-globin) sequence into a mammalian cell. In various embodiments, for delivery to cells, vectors described herein are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

The recombinant LVs and resulting virus described herein are capable of transferring a nucleic acid (e.g., a nucleic acid encoding an anti-sickling β-globin or other sequence) into a mammalian cell. For delivery to cells, various vectors described herein are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

In certain embodiments the vectors are introduced via transfection into the packaging cell line. The packaging cell line produces viral particles that contain the vector genome. Methods for transfection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with or without a dominant selectable marker, such as neomycin, DHFR, Glutamine synthetase, followed by selection in the presence of the appropriate drug and isolation of clones. In certain embodiments the selectable marker gene can be linked physically to the packaging genes in the construct.

Stable cell lines wherein the packaging functions are configured to be expressed by a suitable packaging cell are known (see, e.g., U.S. Pat. No. 5,686,279, which describes packaging cells). In general, for the production of virus particles, one may employ any cell that is compatible with the expression of lentiviral Gag and Pol genes, or any cell that can be engineered to support such expression. For example, producer cells such as 293T cells and HT1080 cells may be used.

The packaging cells with a lentiviral vector incorporated therein form producer cells. Producer cells are thus cells or cell-lines that can produce or release packaged infectious viral particles carrying the therapeutic gene of interest (e.g., modified β-globin). These cells can further be anchorage dependent which means that these cells will grow, survive, or maintain function optimally when attached to a surface such as glass or plastic. Some examples of anchorage dependent cell lines used as lentiviral vector packaging cell lines when the vector is replication competent are HeLa or 293 cells and PERC.6 cells.

Accordingly, in certain embodiments, methods are provided of delivering a gene to a cell which is then integrated into the genome of the cell, comprising contacting the cell with a virion containing a lentiviral vector described herein. The cell (e.g., in the form of tissue or an organ) can be contacted (e.g., infected) with the virion ex vivo and then delivered to a subject (e.g., a mammal, animal or human) in which the gene (e.g., anti-sickling β-globin) will be expressed. In various embodiments the cell can be autologous to the subject (i.e., from the subject) or it can be non-autologous (i.e., allogeneic or xenogenic) to the subject. Moreover, because the vectors described herein are capable of being delivered to both dividing and non-dividing cells, the cells can be from a wide variety including, for example, bone marrow cells, mesenchymal stem cells (e.g., obtained from adipose tissue), and other primary cells derived from human and animal sources. Alternatively, the virion can be directly administered in vivo to a subject or a localized area of a subject (e.g., bone marrow).

Of course, as noted above, the lentivectors described herein will be particularly useful in the transduction of human hematopoietic progenitor cells or a hematopoietic stem cells, obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ hematopoietic stem and progenitor cells.

### Gene Therapy.

In still other embodiments, methods are provide for transducing a human hematopoietic stem cell. In certain embodiments the methods involve contacting a population of human cells that include hematopoietic stem cells with one of the foregoing lentivectors under conditions to effect the transduction of a human hematopoietic progenitor cell in said population by the vector. The stem cells may be transduced in vivo or in vitro, depending on the ultimate application. Even in the context of human gene therapy, such as gene therapy of human stem cells, one may transduce the stem cell in vivo or, alternatively, transduce in vitro followed by infusion of the transduced stem cell into a human subject. In one aspect of this embodiment, the human stem cell can be removed from a human, e.g., a human patient, using methods well known to those of skill in the art and transduced as noted above. The transduced stem cells are then reintroduced into the same or a different human.

Upregulation of Fetal Hemoglobin Isoforms Using shmiRs

β-hemoglobinopathies encompass a number of anemias of genetic origin in which there is a decreased production and/or increased destruction (hemolysis) of red blood cells (RBCs). These also include genetic defects that result in the production of abnormal hemoglobins (such as sickle hemoglobin) leading to abnormal polymerization of the sickle globin molecules with a propensity to damage the red cell membrane, lead to vessel occlusion and a concomitant impaired ability to maintain oxygen concentration. Some such disorders involve the failure to produce normal β-globin in sufficient amounts (e.g., β-thalassemias), while others involve the failure to produce normal β-globin entirely. This leads to an imbalance of alpha and beta chains, damage, and premature destruction of the red blood cells.

The search for treatment aimed at reduction of sickle globin polymerization and globin chain imbalance in patients with β-hemoglobinopathies has focused on the pharmacologic manipulation of fetal HbF. The therapeutic potential of such approaches is demonstrated by observations that certain populations of adult patients with R chain abnormalities and higher than normal levels of HbF experience a milder clinical course of disease than patients with normal adult levels of HbF.

BCL11A is a validated therapeutic target for reactivation of γ-globin gene and therefore HbF expression in the major hemoglobinopathies, sickle cell disease (SCD) and β-thalassemias. Down modulation or genetic deletion of BCL11A relieves γ-globin repression and inactivation of BCL11A in the erythroid lineage prevents SCD phenotype and organ toxicities in genetically engineered mice. The mouse embryonic Hbb-y gene is a functional homolog of the human γ-globin gene, and therefore serves as a convenient surrogate for assessment of the effect of BCL11A knockdown in murine erythroleukemia (MEL) cells.

In certain embodiments, the vectors described herein comprising BCL11A and/or ZNF410 shmiRs can be used to manipulate the levels of fetal hemoglobin (HbF) to compensate for defective adult hemoglobin proteins in subjects with a hemoglobinopathy. As used herein, the term “increasing fetal hemoglobin levels” in a cell or subject indicates that HbF is at least 5% higher in cell populations or in a biological sample obtained from a subject treated with a vector comprising a BCL11A and/or ZNF410 shmiR construct, than in a comparable, control population or subject, where either no vector is administered, or an empty vector lacking the one or more shmiRs is administered. In some embodiments, the percentage of HbF expression in a vector/shmiR construct treated population or subject is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a control treated population of comparable size and culture conditions or as compared to the level of fetal hemoglobin in a given subject prior to treatment with the vector comprising the shmiR construct. The term “control treated cell population” is used herein to describe a population of cells that has been treated with identical media, viral induction, nucleic acid sequences, temperature, confluency, flask size, pH, etc., with the exception of a shmiR construct.

In various embodiments, the methods and compositions described herein utilize miRNA frameworks or regions to flank an shRNA sequence directed against BCL11A and/or ZNF410. The use of such miRNA frameworks permits exploitation of the microRNA-biogenesis pathway to generate shRNAs or siRNAs that target expression of the target gene (e.g., BCL11A or ZNF410). The lentiviral vectors described herein are engineered to express shRNAs in an miRNA framework that mimics primary microRNAs (pri-miRNAs) and are sequentially processed by the endogenous Microprocessor and Dicer complexes to generate small interfering RNAs (siRNAs) or short hairpin RNAs (shRNAs) with sequence complementarity to the BCL11A, or ZNF410 messenger RNA (mRNA).

As used herein, the term “miRNA framework regions” refers to nucleic acid sequences derived from an endogenous miRNA that can be placed upstream and/or downstream of the shRNA and/or in the loop region of an shRNA to generate a shmiR construct as that term is used herein.

It is noted that when a plurality of shmiRs (e.g., 2, 3, or more) are part of a single construct, the miRNA frameworks for each shmiR should be different such that homologous recombination does not occur, thereby preventing proper function of each shmiR.

In one illustrative embodiment, the BCL11A sequence in an miR223 framework comprises the nucleotide sequence: GATCTCACTTCCCCACAGAAGCTCTTGGCCTGGCCTCCTGCAGTGCCACGCTGCGCGATCGAGTGTTGAATAA [symbol] TTATTCAACACTCGATCGCGCAGTGCG GCACATGCTTACCAGCTCTAGGCCAGGGCAGATGGGATATGACGAATGGAC TGCCAGCTGGATACAAGGATGCTCACC (SEQ ID NO:8), where the bold underlined text is the miR223 backbone, the italicized text is the BCL11A guide strand sequence, the dotted, underlined lower case text is the miR223 loop sequence, and the italics, double underlined text is the BCL11A passenger strand sequence.

In one illustrative embodiment, the ZNF410 sequence in an miR144 framework comprises the nucleotide sequence: cgcttttcaagccatgcttcctgtgcccccatggggccctggctGCTGAGCACTTAGTGT TTGTA [symbol] TACAAACACTAAGTGCTCAGCagtccgggcacccccagct ctggagcctgacaaggaggacaggagagat (SEQ ID NO:10) where the bold underlined text is the miR144 backbone, the italicized text is the ZNF410 guide strand sequence, the dotted underlined lower case text is the miR144 loop sequence, and the italics double underlined text is the ZNF410 passenger strand sequence.

As an illustrative, but non-limiting example of a method of treatment of a subject or reducing the risk of developing a hemoglobinopathy in a subject, the method comprises administering to the subject a lentiviral composition as described herein and comprising one or more shmiRs (e.g., BCL11A and/or ZNF410 shmiRs). In one embodiment, the method further comprises identifying a subject as having a hemoglobinopathy, or as at risk of developing a hemoglobinopathy prior to administering a treatment as described herein. In another embodiment, the method further comprises selecting the identified subject having a hemoglobinopathy or as at risk of developing a hemoglobinopathy prior to administering a lentiviral vector comprising one or more shmiRs as described herein.

### Stem Cell/Progenitor Cell Gene Therapy.

In various embodiments the lentivectors described herein are particularly useful for the transduction of human hematopoietic progenitor cells or hematopoietic stem cells (HSCs), obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ hematopoietic stem and progenitor cells.

When cells, for instance CD34⁺ cells, dendritic cells, peripheral blood cells or tumor cells are transduced ex vivo, the vector particles are incubated with the cells using a dose generally in the order of between 1 to 50 multiplicities of infection (MOI) which also corresponds to 1×10⁵ to 50×10⁵ transducing units of the viral vector per 10⁵ cells. This can include amounts of vector corresponding to 1, 2, 3, 4, 5, 6, 7, 8, 9, 10, 15, 20, 25, 30, 35, 40, 45, and 50 MOI. Typically, the amount of vector may be expressed in terms of HT-29 transducing units (TU).

In certain embodiments cell-based therapies involve providing stem cells and/or hematopoietic precursors, transduce the cells with the lentivirus encoding, e.g., an anti-sickling human β-globin, and then introduce the transformed cells into a subject in need thereof (e.g., a subject with the sickle cell mutation).

In certain embodiments the methods involve isolating population of cells, e.g., stem cells from a subject, optionally expand the cells in tissue culture, and administer the lentiviral vector whose presence within a cell results in production of an anti-sickling β-globin in the cells in vitro. The cells are then returned to the subject, where, for example, they may provide a population of red blood cells that produce the anti-sickling R globin.

In some illustrative, but non-limiting, embodiments, a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

Where stem cells are to be used, it will be recognized that such cells can be derived from a number of sources including bone marrow (BM), cord blood (CB), mobilized peripheral blood stem cells (mPBSC), and the like. In certain embodiments the use of induced pluripotent stem cells (IPSCs) is contemplated. Methods of isolating hematopoietic stem cells (HSCs), transducing such cells and introducing them into a mammalian subject are well known to those of skill in the art.

In certain embodiments a lentiviral vector described herein (see, e.g., FIGS. 1 and 4) is used in stem cell gene therapy for SCD by introducing the βAS3 anti-sickling β-globin gene into the bone marrow stem cells of patients with sickle cell disease followed by autologous transplantation.

### Direct Introduction of Vector.

In certain embodiments direct treatment of a subject by direct introduction of the vector(s) described herein is contemplated. The lentiviral compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include inhalation, parenteral, and transmucosal.

In various embodiments pharmaceutical compositions can include an LV in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a lentiviral described herein and/or other agents to be administered together the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a LV calculated to produce the desired therapeutic effect in association with a pharmaceutical carrier.

A unit dose need not be administered as a single injection but may comprise continuous infusion over a set period of time. Unit dose of the LV described herein may conveniently be described in terms of transducing units (T.U.) of lentivector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a LV can include a single treatment or, in many cases, can include a series of treatments.

Illustrative, but non-limiting, doses for administration of gene therapy vectors and methods for determining suitable doses are known in the art. It is furthermore understood that appropriate doses of a LV may depend upon the particular recipient and the mode of administration. The appropriate dose level for any particular subject may depend upon a variety of factors including the age, body weight, general health, gender, and diet of the subject, the time of administration, the route of administration, the rate: of excretion, other administered therapeutic agents, and the like.

In certain embodiments lentiviral gene therapy vectors described herein can be delivered to a subject by, for example, intravenous injection, local administration, or by stereotactic injection (see, e.g., Chen et al. (1994) Proc. Natl. Acad. Sci. USA, 91: 3054). In certain embodiments vectors may be delivered orally or via inhalation and may be encapsulated or otherwise manipulated to protect them from degradation, enhance uptake into tissues or cells, etc. Pharmaceutical preparations can include a LV in an acceptable diluent, or can comprise a slow release matrix in which a LV is imbedded. Alternatively or additionally, where a vector can be produced intact from recombinant cells, as is the case for retroviral or lentiviral vectors as described herein, a pharmaceutical preparation can include one or more cells which produce vectors. Pharmaceutical compositions comprising a LV described herein can be included in a container, pack, or dispenser, optionally together with instructions for administration.

The foregoing compositions, methods and uses are intended to be illustrative and not limiting. Using the teachings provided herein other variations on the compositions, methods and uses will be readily available to one of skill in the art.

The approach to generate reduced length enhance regions is superior to previous strategies for generating tissue-specific enhancers for, among other reasons: 1) The cost of goods is decreased due to a low number of outputs required to be tested, 2) Strength of synthetic enhancers may be superior to those produced with current methods, or they may be less active but more suitable for LV-mediated delivery, and 3). Enhancers can be of minimal length.

Additionally, without being bound to a particular theory, it is believed the enhancer mapping strategy described herein can be modified to generate genome-wide enhancer maps using a similar cloning strategy and sonicated human genomic DNA and that the mapping strategies can be used to generate synthetic enhancers responsive to an array of distinct cellular perturbations.

### EXAMPLES

The following examples are offered to illustrate, but not to limit the claimed invention.

### Example 1

### Evaluation of LV Comprising βAS3 and BCL11A shmiR in miR223

The goal of this study was to determine the optimal location to insert a BCL11A shRNAmiR (shmiR) in a lentiviral vector (LV) to maintain βAS3-globin expression while also increasing fetal globin expression. The beta-globin vector used for this study (designated UV+EC1, also referred to as Mini-G) includes core HS1, HS2, HS3, and HS4 regions.

The vector used for this study is schematically illustrated in FIG. 1, panel A and comprises a β^{AS3}-globin cassette in reverse orientation, beta-globin ENCODE core enhancer sequences (LCR), including HS1, HS2, HS3, and HS4 (see Table 1 for sequences), and a minimized LV backbone.

A BCL11A shRNA imbedded in a microRNA (shmiR) (e.g., microRNA 223 (MIR223)) was inserted into 5 different locations:
1) at the start of IVS1 (position A in FIG. 1, panel B) illustrated in Table 5;
2) at the end of IVS1 (position B in FIG. 1, panel B) illustrated in Table 6;
3) at the deletion in IVS2 (position C in FIG. 1, panel B) illustrated in Table 7; and
4) at the end of IVS2 (position D in FIG. 1, panel B) illustrated in Table 4; and in the 3′ UTR (position E in FIG. 1, panel B) illustrated in Table 8.

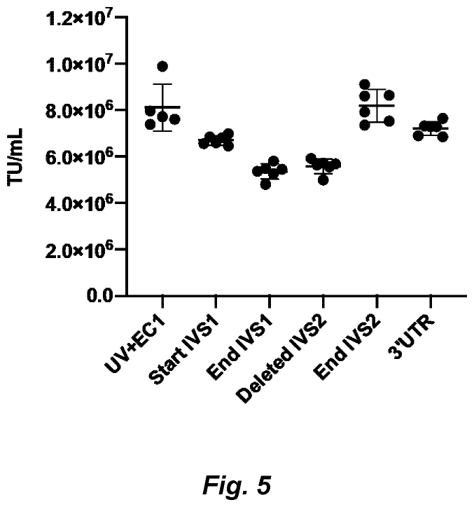

FIG. 5 shows that incorporation of shRNA^{miR} (shmiR) into the LV does not severely impact titer.

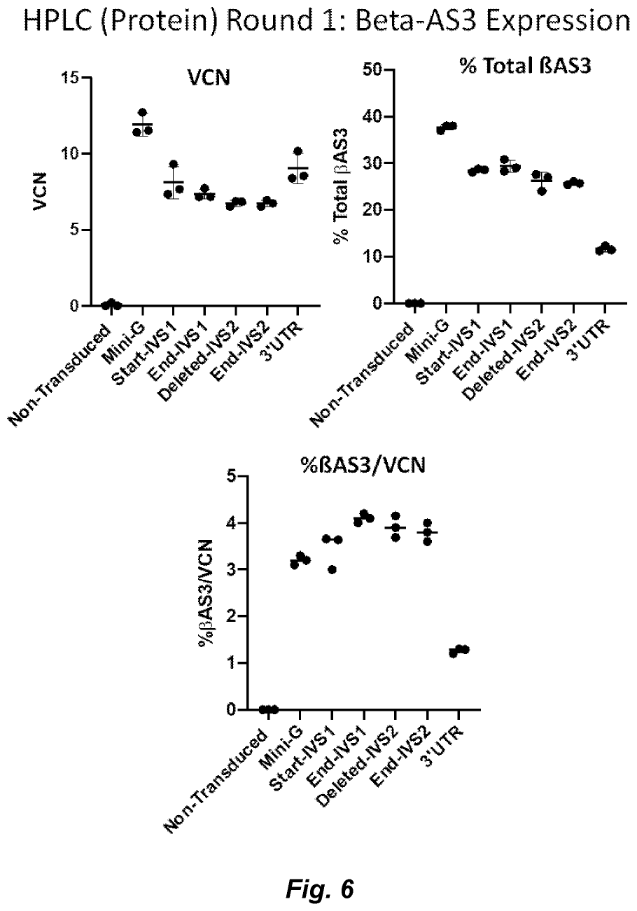

FIGS. 6 (round 1) and 9 (round 2) illustrate beta-AS3 expression by cells transfected with the LV comprising a 3-AS3 gene and a BCL11A shRNA. As shown therein, the vectors express significant levels of 3-AS3 although incorporation of the shRNA appears to decrease 3-AS3 expression somewhat. It is noted that the vector with the shRNA inserted in the 3′UTR showed significantly lower expression.

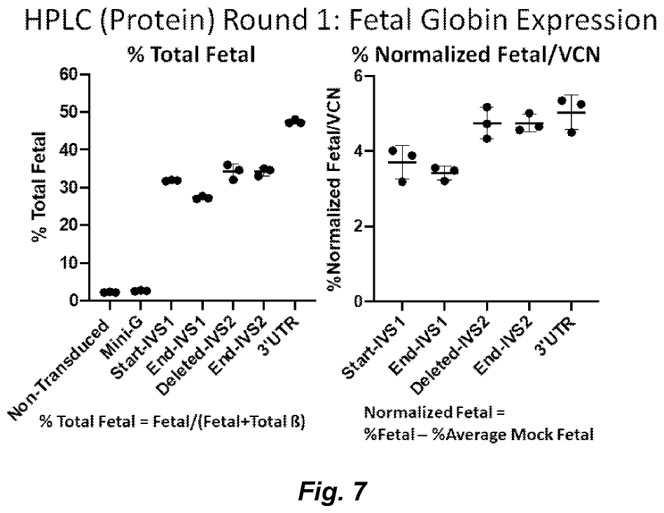

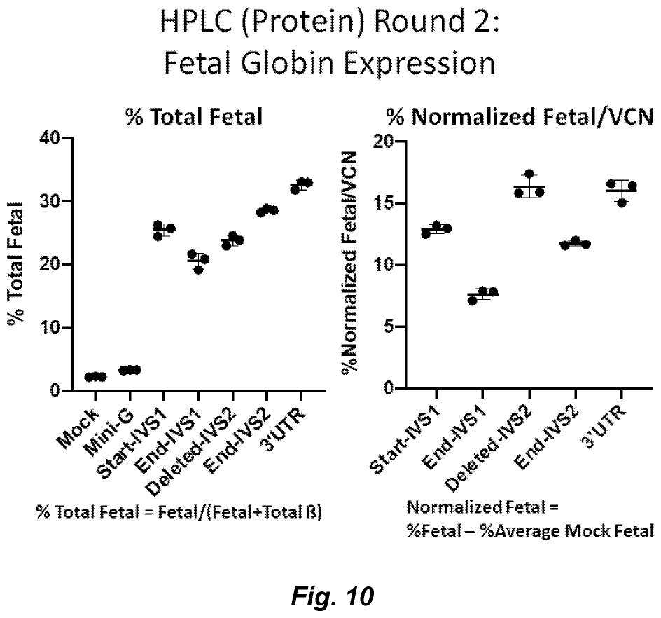

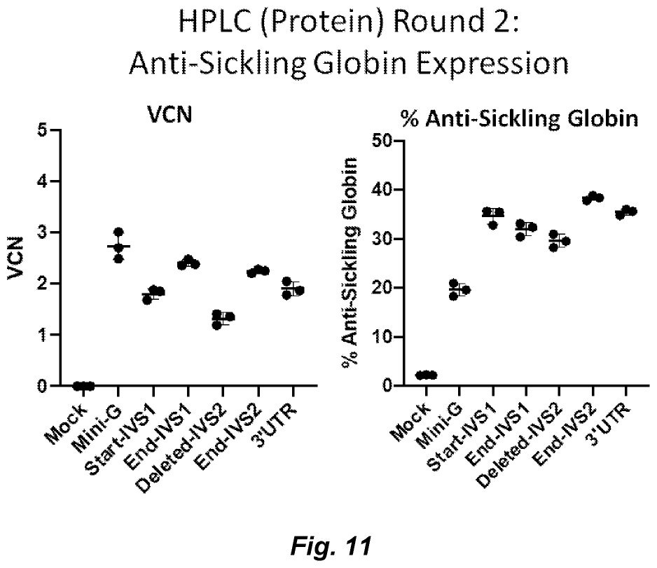

FIGS. 7 and 10 (round 2) illustrate fetal globin expression by cells transfected with the LV comprising a β-AS3 gene and a BCL11A shRNA. As shown therein, all vectors containing the BCL11A shRNA showed increased levels of fetal globin expression.

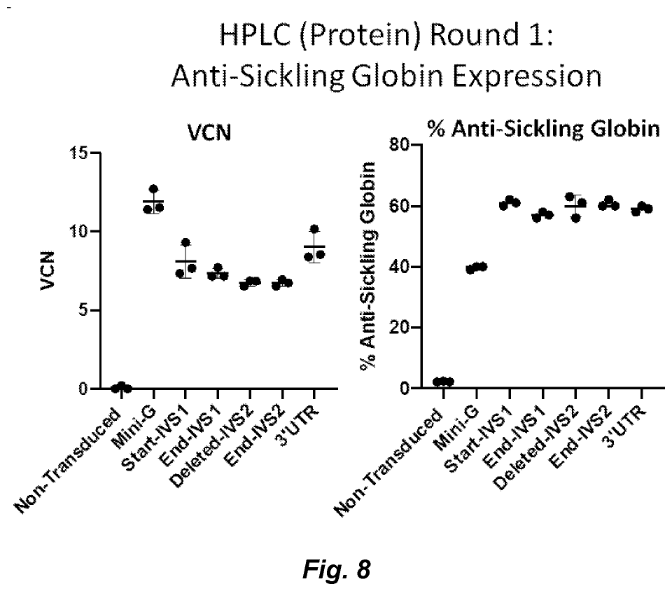

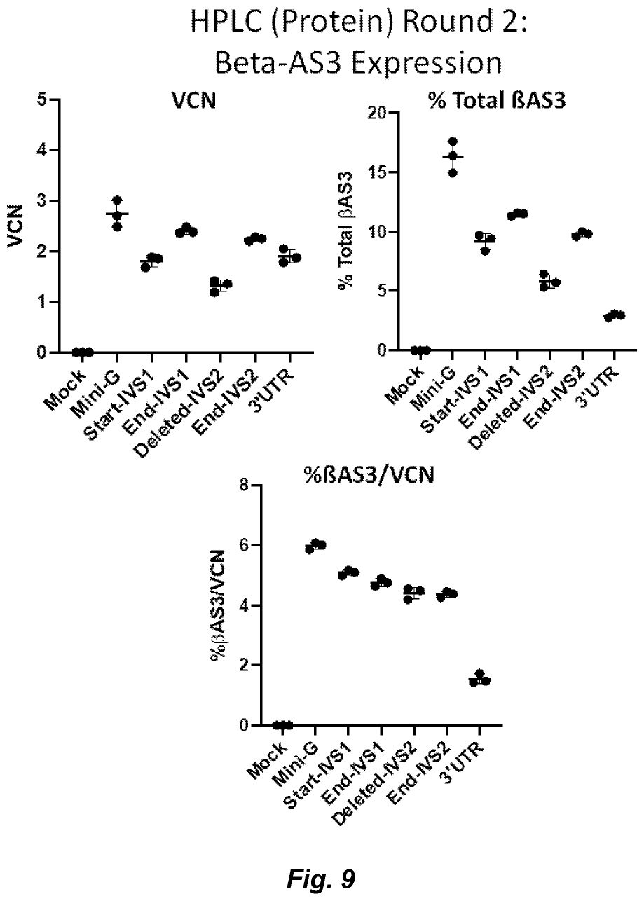

FIGS. 8 (round 1) and 11 (round 2) illustrate anti-sickling globin expression by cells transfected with the LV comprising a β-AS3 gene and a BCL11A shRNA. All of the vectors showed increased levels of anti-sickling globin expression.

TABLE 4
Nucleic acid sequence of Mini-G end IVS2 shmiR lentiviral vector (9555 bp)
(SEQ ID NO: 12). shmiR RNA is inserted at position D in FIG. 1, panel B.

| | |
|---|---|
| acgcgccctgtagcggcgcattaagcgcggcgggtgtggtggttacgcgcagcgtgaccgctacacttg | RRE |
| ccagcgccctagcgcccgctcctttcgctttcttcccttcctttctcgccacgttcgccggctttccccgtcaa | BCL11A |
| gctctaaatcgggggctccctttagggttccgatttagtgctttacggcacctcgaccccaaaaaacttgatt | shmiR in |
| agggtgatggttcacgtagtgggccatcgccctgatagacggtttttcgccctttgacgttggagtccacgtt | miR223 |
| ctttaatagtggactcttgttccaaactggaacaacactcaaccctatctcggtctattcttttgatttataaggg | EC1 |
| attttgccgatttcggcctattggttaaaaaatgagctgatttaacaaaaatttaacgcgaattttaacaaaatat | EC2 |
| taacgcttacaatttaggtggcacttttcggggaaatgtgcgcggaacccctatttgtttatttttctaaatacatt | EC3 |
| caaatatgtatccgctcatgagacaataaccctgataaatgcttcaataatagcacctagatcaagagacag | EC4 |
| gatgaggatcgtttcgcatgattgaacaagatggattgcacgcaggttctccggccgcttgggtggagagg | |
| ctattcggctatgactgggcacaacagacaatcggctgctctgatgccgccgtgttccggctgtcagcgca | |
| ggggcgcccggttctttttgtcaagaccgacctgtccggtgccctgaatgaactgcaagacgaggcagcg | |
| cggctatcgtggctggccacgacgggcgttccttgcgcagctgtgctcgacgttgtcactgaagcgggaa | |
| gggactggctgctattgggcgaagtgccggggcaggatctcctgtcatctcaccttgctcctgccgagaaa | |
| gtatccatcatggctgatgcaatgcggcggctgcatacgcttgatccggctacctgcccattcgaccacca | |
| agcgaaacatcgcatcgagcgagcacgtactcggatggaagccggtcttgtcgatcaggatgatctggac | |
| gaagagcatcaggggctcgcgccagccgaactgttcgccaggctcaaggcgagcatgcccgacggcg | |
| aggatctcgtcgtgacccatggcgatgcctgcttgccgaatatcatggtggaaaatggccgcttttctggatt | |
| catcgactgtggccggctgggtgtggcggaccgctatcaggacatagcgttggctacccgtgatattgctg | |
| aagagcttggcggcgaatgggctgaccgcttcctcgtgctttacggtatcgccgctcccgattcgcagcgc | |
| atcgccttctatcgccttcttgacgagttcttctgaattattaacgcttacaatttcctgatgcggtattttctcctta | |
| cgcatctgtgcggtatttcacaccgcatcaggtggcacttttcggggaaatgtgcgcggaacccctatttgtt | |
| tatttttctaaatacattcaaatatgtatccgctcatgaccaaaatcccttaacgtgagttttcgttccactgagc | |
| gtcagaccccgtagaaaagatcaaaggatcttcttgagatcctttttttctgcgcgtaatctgctgcttgcaaa | |
| caaaaaaaccaccgctaccagcggtggtttgtttgccggatcaagagctaccaactctttttccgaaggtaa | |
| ctggcttcagcagagcgcagataccaaatactgttcttctagtgtagccgtagttaggccaccacttcaaga | |
| actctgtagcaccgcctacatacctcgctctgctaatcctgttaccagtggctgctgccagtggcgataagtc | |
| gtgtcttaccgggttggactcaagacgatagttaccggataaggcgcagcggtcgggctgaacgggggg | |
| gtcgtgcacacagcccagcttggagcgaacgacctacaccgaactgagatacctacagcgtgagctatg | |
| agaaagcgccacgcttcccgaagggagaaaggcggacaggtatccggtaagcggcagggtcggaaca | |
| ggagagcgcacgagggagcttccagggggaaacgcctggtatctttatagtcctgtcgggtttcgccacc | |
| tctgacttgagcgtcgatttttgtgatgctcgtcaggggggcggagcctatggaaaaacgccagcaacgc | |
| ggcctttttacggttcctggccttttgctggccttttgctcacatgttctttcctgcgttatcccctgattctgtgga | |
| taaccgtattaccgcctttgagtgagctgataccgctcgccgcagccgaacgaccgagcgcagcgagtca | |
| gtgagcgaggaagcggaagagcgcccaatacgcaaaccgcctctccccgcgcgttggccgattcattaa | |
| tgcagctggcacgacaggtttcccgactggaaagcgggcagtgagcgcaacgcaattaatgtgagttagc | |
| tcactcattaggcaccccaggctttacactttatgcttccggctcgtatgttgtgtggaattgtgagcggataa | |
| caatttcacacaggaaacagctatgaccatgattacgccaagcgcgcaattaaccctcactaaagggaaca | |
| aaagctggagctgcaagcttggccattgcatacgttgtatccatatcataatatgtacatttatattggctcatg | |
| tccaacattaccgccatgttgacattgattattgactagttattaatagtaatcaattacggggtcattagttcata | |
| gcccatatatggagttccgcgttacataacttacggtaaatggcccgcctggctgaccgcccaacgacccc | |
| cgcccattgacgtcaataatgacgtatgttcccatagtaacgccaatagggactttccattgacgtcaatggg | |
| tggagtatttacggtaaactgcccacttggcagtacatcaagtgtatcatatgccaagtacgccccctattga | |
| cgtcaatgacggtaaatggcccgcctggcattatgcccagtacatgaccttatgggactttcctacttggca | |
| gtacatctacgtattagtcatcgctattaccatggtgatgcggttttggcagtacatcaatgggcgtggatagc | |
| ggtttgactcacggggatttccaagtctccaccccattgacgtcaatgggagtttgttttggcaccaaaatca | |
| acgggactttccaaaatgtcgtaacaactccgccccattgacgcaaatgggcggtaggcgtgtacggtgg | |
| gaggtctatataagcagagctcgtttagtgaaccgGGGTCTCTCTGGTTAGACCAGAT | |
| CTGAGCCTGGGAGCTCTCTGGCTAACTAGGGAACCCACTGCTTA | |
| AGCCTCAATAAAGCTTGCCTTGAGTGCTTCAAGTAGTGTGTGC | |
| CCGTCTGTTGTGTGACTCTGGTAACTAGAGATCCCTCAGAC | |
| CCTTTTAGTCAGTGTGGAAAATCTCTAGCagtggcgcccgaacagggact | |
| tgaaagcgaaagggaaaccagaggagctctcTCGACGCAGGACTCGGCTTGCTG | |
| AAGCGCGCACGGCAAGAGGCGAGGGGCGGCGACTGGTGAGTAC | |
| GCCAAAAATTTTGACTAGCGGAGGCTAGAAGGAGAGAGATGGG | |
| TGCGAGAGCGTCAGTATTAAGCGGGGGAGaattagatcgcgatgggaaaaaa | |
| ttcggttaaggccagggggaaagaaaaaatataaattaaaacatatagtatgggcaagcagggagctaga | |
| acgattcgcagttaatcctggcctgttagaaacatcagaaggctgtagacaaatactgggacagctacaac | |
| catcccttcagacaggatcagaagaacttagatcattatataatacagtagcaaccctctattgtgtgcatcaa | |
| aggatagagataaaagacaccaaggaagctttagacaagatagaggaagagcaaaacaaaagtaagac | |
| caccgcacagcaagcGGCCGCTgatcttcagacctggaggaggagatatgagggacaattggag | |
| aagtgaattatataaatataaagtagtaaaaattgaaccattagg | |
| agtagcacccaccaaggcaaagagaagagtggtgcagagagaaaaaagaGCAGTGGGAATa | |
| ggagctttgttccttgggttcttgggagcagcaggaagcactatgggcgcagcgtcaatgacgctgacggt | |
| acaggccagacaattattgtctggtatagtgcagcagcagaacaatttgctgagggctattgaggcgcaac | |
| agcatctgttgcaactcacagtctggggcatcaagcagctccaggcaagaat | |
| cctggctgtggaaagatacctaaaggatcaacagctcctggggatttggggttgctctggaaaactcatttg | |
| caccactgctgtgccttggaatgctagttggagtaataaatctctggaacagatttggaatcacacgacctg | |
| gatggagtgggacagagaaattaacaattacacaagcttaatacactccttaattgaagaatcgcaaaacca | |
| gcaagaaaagaatgaacaagaattattggaattagataaatgggcaagtttgtggaattggtttaacataaca | |
| aattggctgtggtatataaaattattcataatgatagtaggaggcttggtaggtttaagaatagtttttgctgtac | |
| tttctatagtgaatagagttaggcagggatattcaccattatcgtttcagacccacctcccaaccccgagggg | |
| acccgacaggcccgaaggaatagaagaagaaggtggagagagagacagagacagatccattcgatta | |
| gtgaacggatCTCGACGGTATCGATCTCGAcacaaatggcagtattcatccacaattt | |
| taaaagaaaaggggggattggggggtacagtgcaggggaaagaatagtagacataatagcaac | |
| agacatacaaactaaagaattacaaaaacaaattacaaaaattcaaaattttcgggtttattacagg | |
| gacagcagacatgaggacagctaaaacaataagtaatgtaaaatacagcatagcaaaactttaac | |
| ctccaaatcaagcctctacttgaatccttttctgagggatgaataaggcataggcatcaggggctgtt | |
| gccaatgtgcattagctgtttgcagcctcaccttctttcatggagtttaagatatagtgtattttcccaa | |
| ggtttgaactagctcttcatttctttatgttttaaatgcactgacctcccacattccctttttagtaaaatatt | |
| cagaaataatttaaatacatcattgcaatgaaaataaatgttttttattaggcagaatccagatgctcaaggcc | |
| cttcataatatcccccagtttagtagttggacttagggaacaaaggaacctttaatagaaattggacagcaag | |
| aaagcgagcttagtgatacttgtgggccagggcattagccacaccagccaccactttctgataggcagcct | |
| gcactggtggggtgaattctttgccaaagtgatgggccagcacacagaccagcacgttgcccaggagctg | |
| tgggaggaagataagag | |
| GGTGAGCATCCTTGTATCCAGCTGGCAGTCCATTCGTCATATCC | |
| CATCTGCCCTGGCCTAGAGCTGGTAAGCATGTGCCGCACTGCGC | |
| GATCGAGTGTTGAATAActctaccacatggagTTATTCAACACTCGATCG | |
| CGCAGCGTGGCACTGCAGGAGGCCAGGCCAAGAGCTTCTGTGG | |
| GGAAGTGAGATC | |
| Gtatgaacatgattagcaaaagggcctagcttggactcagaataatccagccttatcccaaccataaaataa | |
| aagcagaatggtagctggattgtagctgctattagcaatatgaaacctcttacatcagttacaatttatatgca | |
| gaaataccctgttacttctccccttcctatgacatgaacttaaccatagaaaagaaggggaaagaaaacatc | |
| aagggtcccatagactcaccctgaagttctcaggatccacgtgcagcttgtcacagtgcagctcactcagct | |
| gggcaaaggtgcccttgaggttgtccaggtgagccaggccatcactaaaggcaccgagcactttcttgcc | |
| atgagccttcaccttagggttgcccataacagcatcaggagtggacagatccccaaaggactcaaagaac | |
| ctctgggtccaagggtagaccaccagcagcctaaggggggaaaatagaccaataggcagagagagtc | |
| agtgcctatcagaaacccaagagtcttctctgtctccacatgcccagtttctattggtctccttaaacctgtctt | |
| gtaaccttgataccaacctgcccagggcctcaccaccaacggcatccacgttcaccttgtcccacagggc | |
| agtaacggcagacttctcctcaggagtcaggtgcaccatggtgtctgtttgaggttgctagtgaacacagtt | |
| gtgtcagaagcaaatgtaagcaatagatggctctgccctgacttttatgcccagccctggctcctgccctcc | |
| ctgctcctgggagtagattggccaaccctagggtgtggctccacagggtgaggtctaagtgatgacagcc | |
| gtacctgtccttggctcttctggcactggcttaggagttggacttcaaaccctcagccctccctctaagatata | |
| tctcttggccccataccatcagtacaaattgctactaaaaacatcctcctttgcaagtgtatttac | |
| Catcaataattctagccccacaggagtttgttctgaaagtaaacttccacaaccgcaagcttattgagg | |
| ctaaggcatctgtgaaggaaagaaacatctcctctaaaccactatgctgctagagcctcttttctgtactc | |
| aagcctcattcagacactagtgtcaccagtctcctcatatacctattgtattttcttcttcttgctggtttagtc | |
| atgttttctgggagcttaggggcttattttattttgttttgttttctaatcaacagagatgggcaaacccattatt | |
| tttttctttagacttgggatggtgatagctgggcagcgtcagaaactgtgtgtggatatagataagagctc | |
| aggactatgctgagctgtgatgagggaggggcctagctaaaggcagtgagagtcagaatgctcctgc | |
| tattgccttctcagtccccacgcttggtttctacacaagtagatacatagaaaaggctataggttagtgttt | |
| gagagtcctgcatgattagttgctcagaaatgcccgataaatatgttatgtgtgtttatgtatatatatgtttt | |
| atatgtgtgtgtgtgtgtgttgtgtttacaaatatgtgattatcatcaaaacgtgaggg | |
| TACGTaTATGTGTATATATATATATATATTCAGGAAATAATATATTCTAG | |
| AATATGTCACATTCTGTCTCAGGCATCCATTTTCTTTATGATGCCGTTT | |
| GAGGTGGAGTTTTAGTCAGGTGGTCAGCTTCTCCTTTTTTTTGCCATC | |
| TGCCCTGTAAGCATCCTGCTGGGGACCCAGATAGGAGTCATCACTC | |
| TAGGCTGAGAACATCTGGGCACACACCCTAAGCCTCAGCATGACTC | |
| ATCATGACTCAGCATTGCTGTGCTTGAGCCAGAAGGTTTGCTTAGAA | |
| GGTTACACAGAACCAGAAGGCGGGGGTGGGGCACTGACCCCGACA | |
| GGGGCCTGGCCAGAACTGCTCATGCTTGGACTATGGGAGGTCACTA | |
| ATGGAGACACACAGAAATGTAACAGGAACTAAGGAAAAACTGAAGCT | |
| T | |
| Tgggggtataggggagcagtcccatgtagtagtagaatgaaaaatgctgctatgctgtgcctcccccacct | |
| ttcccatgtctgccctctactcatggtctatctctcctggctcctgggagtcatggactccacccagcaccac | |
| caacctgacctaaccacctatctgagcctgccagcctataacccatctgggccctgatagctggtggccag | |
| ccctgaccccaccccaccctccctggaacctctgatagacacatctggcacaccagctcgcaaagtcacc | |
| gtgagggtcttgtgtttgctgagtcaaaattccttgaaatccaagtccttagagactcc | |
| Caggcttggattcaaagctcctgactttctgtctagtgtatgtgcagtgagccccttttcctctaactgaaaga | |
| aggaaaaaaaaatggaacccaaaatattctacatagtttccatgtcacagccagggctgggcagtctcctgt | |
| tatttcttttaaaataaatatatcatttaaatgcataaataagcaaaccctgctcgggaatgggagggagagtc | |
| tctggagtccaccccttctcggccctggctctgcagatagtgctatcaaagccctgacagagccctgcccat | |
| tgctgggccttggagtgagtcagcctagtagagaggcagggcaagccatctcatagctgctgagtggga | |
| gagagaaaagggctcattgtctataaactcaggtcatggctattcttat | |
| taaaagaaaaggggggactggaagggctaattcactcccaacgaagacaagatctgctttttgcttgtact | |
| GGGTCTCTCTGGTTAGACCAGATCTGAGCCTGGGAGCTCTCTGG | |
| CTAACTAGGGAACCCACTGCTTAAGCCTCAATAAAGCTTGCCTT | |
| GAGTGCTTCAAGTAGTGTGTTGGTTTTTTGTGTGTGCATTGTCTG | |
| AGTAGGTGTCATTCTATTCTGGGGGGTGGGGTGGGGCAGCACA | |
| GCAAGGGGGAGGATTGGGAAGACAATAGCAGGCATGCTGGGG | |
| ATGCGGTGGGCTCTATGGagtagtagttcatgtcatcttattattcagtatttataacttgcaaa | |
| gaaatgaatatcagagagtgagaggaacttgtttattgcagcttataatggttacaaataaagcaatagcatc | |
| acaaatttcacaaataaagcatttttttcactgcattctagttgtggtttgtccaaactcatcaatgtatcttatcat | |
| gtctggctctagctatcccgcccctaactccgcccATCCCGCCCCTAACTCCGCCCAG | |
| TTCCGCCCATTCTCCGCCCCATGGCTCTCACTACTTCTGGAATAG | |
| CTCAGAGGCCGAGGCGGCCTCGGCCTCTGCATAAATAAAAAAA | |
| ATTAGTCGAGGCTTTTTTGGAGGCCTAGGgacgtacccaattcgccctatagtg | |
| agtcgtattacgcgcgctcactggccgtcgttttacaacgtcgtgactgggaaaaccctggcgttacccaac | |
| ttaatcgccttgcagcacatccccctttcgccagctggcgtaatagcgaagaggcccgcaccgatcgccct | |
| tcccaacagttgcgcagcctgaatggcgaatggg | |

| | |
|---|---|
| XXXXXXXXXX | BCL11A shmiR in miR223 (reverse complement) |
| XXXXXXXXXX | ecCORE HS1 or EC1 |
| XXXXXXXXXX | ecCORE HS2 or EC2 |
| XXXXXXXXXX | ecCORE HS3 or EC3 |
| XXXXXXXXXX | ecCORE HS4 or EC4 |
| XXXXXXXXXX | RRE |
| XXXXXXXXXX | U5 |
| xxxxxxxxx | CPPT |
| XXXXXXXXXX | 3′UTR |

TABLE 5
Nucleic acid sequence of Mini-G vector containing EC1 with shmiR sequence at the
start of IVS1 (Mini-G start IVS1 shmiR lentiviral vector (SEQ ID NO: 13)). shmiR RNA is
inserted at position A in FIG. 1, panel B.

acgcgccctgtagcggcgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttc
gccctttgacgttggagtccacgttctttaatagtggactcttgttccaaactggaacaacactcaaccctatctcggtctattcttttgat
ttataagggattttgccgatttcggcctattggttaaaaaatgagctgatttaacaaaaatttaacgcgaattttaacaaaatattaacgct
tacaatttaggtggcacttttcggggaaatgtgcgcggaacccctatttgtttatttttctaaatacattcaaatatgtatccgctcatgag
acaataaccctgataaatgcttcaataatagcacctagatcaagagacaggatgaggatcgtttcgcatgattgaacaagatggattg
cacgcaggttctccggccgcttgggtggagaggctattcggctatgactgggcacaacagacaatcggctgctctgatgccgccg
tgttccggctgtcagcgcaggggcgcccggttctttttgtcaagaccgacctgtccggtgccctgaatgaactgcaagacgaggca
gcgcggctatcgtggctggccacgacgggcgttccttgcgcagctgtgctcgacgttgtcactgaagcgggaagggactggctg
ctattgggcgaagtgccggggcaggatctcctgtcatctcaccttgctcctgccgagaaagtatccatcatggctgatgcaatgcgg
cggctgcatacgcttgatccggctacctgcccattcgaccaccaagcgaaacatcgcatcgagcgagcacgtactoggatggaag
ccggtcttgtcgatcaggatgatctggacgaagagcatcaggggctcgcgccagccgaactgttcgccaggctcaaggcgagca
tgcccgacggcgaggatctcgtcgtgacccatggcgatgcctgcttgccgaatatcatggtggaaaatggccgcttttctggattca
tcgactgtggccggctgggtgtggcggaccgctatcaggacatagcgttggctacccgtgatattgctgaagagcttggcggcga
atgggctgaccgcttcctcgtgctttacggtatcgccgctcccgattcgcagcgcatcgccttctatcgccttcttgacgagttcttctg
aattattaacgcttacaatttcctgatgcggtattttctccttacgcatctgtgcggtatttcacaccgcatcaggtggcacttttcgggga
aatgtgcgcggaacccctatttgtttatttttctaaatacattcaaatatgtatccgctcatgaccaaaatcccttaacgtgagttttcgttc
cactgagcgtcagaccccgtagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggcttcagcagagcgcagat
accaaatactgttcttctagtgtagccgtagttaggccaccacttcaagaactctgtagcaccgcctacatacctcgctctgctaatcct
gttaccagtggctgctgccagtggcgataagtcgtgtcttaccgggttggactcaagacgatagttaccggataaggcgcagcggt
cgggctgaacgggggggtcgtgcacacagcccagcttggagcgaacgacctacaccgaactgagatacctacagcgtgagcta
tgagaaagcgccacgcttcccgaagggagaaaggcggacaggtatccggtaagcggcagggtcggaacaggagagcgcacg
agggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttc
tttcctgcgttatcccctgattctgtggataaccgtattaccgcctttgagtgagctgataccgctcgcgcagccgaacgaccgagc
gcagcgagtcagtgagcgaggaagcggaagagcgcccaatacgcaaaccgcctctccccgcgcgttggccgattcattaatgc
agctggcacgacaggtttcccgactggaaagcgggcagtgagcgcaacgcaattaatgtgagttagctcactcattaggcacccc
aggctttacactttatgcttccggctcgtatgttgtgtggaattgtgagcggataacaatttcacacaggaaacagctatgaccatgatt
acgccaagcgcgcaattaaccctcactaaagggaacaaaagctggagctgcaagcttggccattgcatacgttgtatccatatcata
atatgtacatttatattggctcatgtccaacattaccgccatgttgacattgattattgactagttattaatagtaatcaattacggggtcatt
agttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgccc
agtacatgaccttatgggactttcctacttggcagtacatctacgtattagtcatcgctattaccatggtgatgcggttttggcagtacat
caatgggcgtggatagcggtttgactcacggggatttccaagtctccaccccattgacgtcaatgggagtttgttttggcaccaaaat
caacgggactttccaaaatgtcgtaacaactccgccccattgacgcaaatgggcggtaggcgtgtacggtgggaggtctatataag
cagagctcgtttagtgaaccgGGGTCTCTCTGGTTAGACCAGATCTGAGCCTGGGAGCTCTC
TGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAG
ACCCTTTTAGTCAGTGTGGAAAATCTCTAGCagtggcgcccgaacagggacttgaaagcgaaagg
gaaaccagaggagctctcTCGACGCAGGACTCGGCTTGCTGAAGCGCGCACGGCAAGAG
GCGAGGGGCGGCGACTGGTGAGTACGCCAAAAATTTTGACTAGCGGAGGCTA
GAAGGAGAGAGATGGGTGCGAGAGCGTCAGTATTAAGCGGGGGAGaattagatcgcg
atgggaaaaaattcggttaaggccagggggaaagaaaaaatataaattaaaacatatagtatgggcaagcagggagctagaacg
attcgcagttaatcctggcctgttagaaacatcagaaggctgtagacaaatactgggacagctacaaccatcccttcagacaggatc
agaagaacttagatcattatataatacagtagcaaccctctattgtgtgcatcaaaggatagagataaaagacaccaaggaagcttta
gacaagatagaggaagagcaaaacaaaagtaagaccaccgcacagcaagcGGCCGCTgatcttcagacctggaggagg
agatatgagggacaattggagaagtgaattatataaatataaagtagtaaaaattgaaccattaggagtagcacccaccaaggcaa
agagaagagtggtgcagagagaaaaaagaGCAGTGGGAATaggagctttgttccttgggttcttgggagcagcaggaa
gcactatgggcgcagcgtcaatgacgctgacggtacaggccagacaattattgtctggtatagtgcagcagcagaacaatttgctg
agggctattgaggcgcaacagcatctgttgcaactcacagtctggggcatcaagcagctccaggcaagaatcctggctgtggaaa
gatacctaaaggatcaacagctcctggggatttggggttgctctggaaaactcatttgcaccactgctgtgccttggaatgctagttg
gagtaataaatctctggaacagatttggaatcacacgacctggatggagtgggacagagaaattaacaattacacaagcttaataca
ctccttaattgaagaatcgcaaaaccagcaagaaaagaatgaacaagaattattggaattagataaatgggcaagtttgtggaattg
gtttaacataacaaattggctgtggtatataaaattattcataatgatagtaggaggcttggtaggtttaagaatagtttttgctgtactttc
tatagtgaatagagttaggcagggatattcaccattatcgtttcagacccacctcccaaccccgaggggacccgacaggcccgaa
ggaatagaagaagaaggtggagagagagacagagacagatccattcgattagtgaacggatCTCGACGGTATCGA
TCTCGAcacaaatggcagtattcatccacaattttaaaagaaaaggggggattggggggtacagtgcaggggaaagaatagt
agacataatagcaacagacatacaaactaaagaattacaaaaacaaattacaaaaattcaaaattttcgggtttattacagggacagc
agacatgaggacagctaaaacaataagtaatgtaaaatacagcatagcaaaactttaacctccaaatcaagcctctacttgaatccttt
tctgagggatgaataaggcataggcatcaggggctgttgccaatgtgcattagctgtttgcagcctcaccttctttcatggagtttaag
atatagtgtattttcccaaggtttgaactagctcttcatttctttatgttttaaatgcactgacctcccacattccctttttagtaaaatattcag
aaataatttaaatacatcattgcaatgaaaataaatgttttttattaggcagaatccagatgctcaaggcccttcataatatcccccagttt
agtagttggacttagggaacaaaggaacctttaatagaaattggacagcaagaaagcgagcttagtgatacttgtgggccagggca
ttagccacaccagccaccactttctgataggcagcctgcactggtggggtgaattctttgccaaagtgatgggccagcacacagac
cagcacgttgcccaggagctgtgggaggaagataagaggtatgaacatgattagcaaaagggcctagcttggactcagaataatc
cagccttatcccaaccataaaataaaagcagaatggtagctggattgtagctgctattagcaatatgaaacctcttacatcagttacaa
tttatatgcagaaataccctgttacttctccccttcctatgacatgaacttaaccatagaaaagaaggggaaagaaaacatcaagggt
cccatagactcaccctgaagttctcaggatccacgtgcagcttgtcacagtgcagctcactcagctgggcaaaggtgcccttgagg
ttgtccaggtgagccaggccatcactaaaggcaccgagcactttcttgccatgagccttcaccttagggttgcccataacagcatca
ggagtggacagatccccaaaggactcaaagaacctctgggtccaagggtagaccaccagcagcctaaggggggaaaatagac
caataggcagagagagtcagtgcctatcagaaacccaagagtcttctctgtctccacatgcccagtttctattgGGTGAGCA
TCCTTGTATCCAGCTGGCAGTCCATTCGTCATATCCCATCTGCCCTGGCCT
AGAGCTGGTAAGCATGTGCCGCACTGCGCGATCGAGTGTTGAATAActctacc
acatggagTTATTCAACACTCGATCGCGCAGCGTGGCACTGCAGGAGGCCAGG
CCAAGAGCTTCTGTGGGGAAGTGAGATCgtctccttaaacctgtcttgtaaccttgataccaacctgc
ccagggcctcaccaccaacggcatccacgttcaccttgtcccacagggcagtaacggcagacttctcctcaggagtcaggtgcac
catggtgtctgtttgaggttgctagtgaacacagttgtgtcagaagcaaatgtaagcaatagatggctctgccctgacttttatgccca
gccctggctcctgccctccctgctcctgggagtagattggccaaccctagggtgtggctccacagggtgaggtctaagtgatgaca
gccgtacctgtccttggctcttctggcactggcttaggagttggacttcaaaccctcagccctccctctaagatatatctcttggcccca
taccatcagtacaaattgctactaaaaacatcctcctttgcaagtgtatttaccatcaataattctagccccacaggagtttgttctgaaa
gtaaacttccacaaccgcaagcttattgaggctaaggcatctgtgaaggaaagaaacatctcctctaaaccactatgctgctagagc
ctcttttctgtactcaagcctcattcagacactagtgtcaccagtctcctcatatacctattgtattttcttcttcttgctggtttagtcatgtttt
ctgggagcttaggggcttattttattttgttttgttttctaatcaacagagatgggcaaacccattatttttttctttagacttgggatggtgat
agctgggcagcgtcagaaactgtgtgtggatatagataagagctcaggactatgctgagctgtgatgagggaggggcctagctaa
aggcagtgagagtcagaatgctcctgctattgccttctcagtccccacgcttggtttctacacaagtagatacatagaaaaggctata
ggttagtgtttgagagtcctgcatgattagttgctcagaaatgcccgataaatatgttatgtgtgtttatgtatatatatgttttatatgtgtgt
gtgtgtgtgttgtgtttacaaatatgtgattatcatcaaaacgtgagggTACGTaTATGTGTATATATATATATA
TATTCAGGAAATAATATATTCTAGAATATGTCACATTCTGTCTCAGGCATCCAT
TTTCTTTATGATGCCGTTTGAGGTGGAGTTTTAGTCAGGTGGTCAGCTTCTCCTT
TTTTTTGCCATCTGCCCTGTAAGCATCCTGCTGGGGACCCAGATAGGAGTCATC
ACTCTAGGCTGAGAACATCTGGGCACACACCCTAAGCCTCAGCATGACTCATC
ATGACTCAGCATTGCTGTGCTTGAGCCAGAAGGTTTGCTTAGAAGGTTACACA
GAACCAGAAGGCGGGGGTGGGGCACTGACCCCGACAGGGGCCTGGCCAGAAC
TGCTCATGCTTGGACTATGGGAGGTCACTAATGGAGACACACAGAAATGTAAC
AGGAACTAAGGAAAAACTGAAGCTTtgggggtataggggagcagtcccatgtagtagtagaatgaaaaat
gctgctatgtgtgcctcccccacctttcccatgtctgccctctactcatggtctatctctcctggctcctgggagtcatggactccacc
cagcaccaccaacctgacctaaccacctatctgagcctgccagcctataacccatctgggccctgatagctggtggccagccctga
ccccaccccaccctccctggaacctctgatagacacatctggcacaccagctcgcaaagtcaccgtgagggtcttgtgtttgctga
gtcaaaattccttgaaatccaagtccttagagactcccaggcttggattcaaagctcctgactttctgtctagtgtatgtgcagtgagcc
ccttttcctctaactgaaagaaggaaaaaaaaatggaacccaaaatattctacatagtttccatgtcacagccagggctgggcagtct
cctgttatttcttttaaaataaatatatcatttaaatgcataaataagcaaaccctgctcgggaatgggagggagagtctctggagtcca
ccccttctcggccctggctctgcagatagtgctatcaaagccctgacagagccctgcccattgctgggccttggagtgagtcagcct
agtagagaggcagggcaagccatctcatagctgctgagtgggagagagaaaagggctcattgtctataaactcaggtcatggcta
ttcttattaaaagaaaaggggggactggaagggctaattcactcccaacgaagacaagatctgctttttgcttgtactGGGTCT
CTCTGGTTAGACCAGATCTGAGCCTGGGAGCTCTCTGGCTAACTAGGGAACCC
ACTGCTTAAGCCTCAATAAAGCTTGCCTTGAGTGCTTCAAGTAGTGTGTTGGTT
TTTTGTGTGTGCATTGTCTGAGTAGGTGTCATTCTATTCTGGGGGGTGGGGTGG
GGCAGCACAGCAAGGGGGAGGATTGGGAAGACAATAGCAGGCATGCTGGGGA
TGCGGTGGGCTCTATGGagtagtagttcatgtcatcttattattcagtatttataacttgcaaagaaatgaatatcagag
agtgagaggaacttgtttattgcagcttataatggttacaaataaagcaatagcatcacaaatttcacaaataaagcatttttttcactgc
attctagttgtggtttgtccaaactcatcaatgtatcttatcatgtctggctctagctatcccgcccctaactccgcccATCCCGCC
CCTAACTCCGCCCAGTTCCGCCCATTCTCCGCCCCATGGCTCTCACTACTTCTG
GAATAGCTCAGAGGCCGAGGCGGCCTCGGCCTCTGCATAAATAAAAAAAATTA
GTCGAGGCTTTTTTGGAGGCCTAGGgacgtacccaattcgccctatagtgagtcgtattacgcgcgctcact
ggccgtcgttttacaacgtcgtgactgggaaaaccctggcgttacccaacttaatcgccttgcagcacatccccctttcgccagctg
gcgtaatagcgaagaggcccgcaccgatcgcccttcccaacagttgcgcagcctgaatggcgaatggg
XXXXXXXXXX BCL11A shmiR in miR223 (reverse complement)

TABLE 6
Nucleic acid sequence of Mini-G end IVS1 shmiR lentiviral vector (SEQ ID NO: 14).
shmiR RNA is inserted at the IVS1 end position (position B in FIG. 1, panel B).

acgcgccctgtagcggcgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttc
gccctttgacgttggagtccacgttctttaatagtggactcttgttccaaactggaacaacactcaaccctatctcggtctattcttttgat
ttataagggattttgccgatttcggcctattggttaaaaaatgagctgatttaacaaaaatttaacgcgaattttaacaaaatattaacgct
tacaatttaggtggcacttttcggggaaatgtgcgcggaacccctatttgtttatttttctaaatacattcaaatatgtatccgctcatgag
acaataaccctgataaatgcttcaataatagcacctagatcaagagacaggatgaggatcgtttcgcatgattgaacaagatggattg
cacgcaggttctccggccgcttgggtggagaggctattcggctatgactgggcacaacagacaatcggctgctctgatgccgccg
tgttccggctgtcagcgcaggggcgcccggttctttttgtcaagaccgacctgtccggtgccctgaatgaactgcaagacgaggca
gcgcggctatcgtggctggccacgacgggcgttccttgcgcagctgtgctcgacgttgtcactgaagcgggaagggactggctg
ctattgggcgaagtgccggggcaggatctcctgtcatctcaccttgctcctgccgagaaagtatccatcatggctgatgcaatgcgg
cggctgcatacgcttgatccggctacctgcccattcgaccaccaagcgaaacatcgcatcgagcgagcacgtactcggatggaag
ccggtcttgtcgatcaggatgatctggacgaagagcatcaggggctcgcgccagccgaactgttcgccaggctcaaggcgagca
tgcccgacggcgaggatctcgtcgtgacccatggcgatgcctgcttgccgaatatcatggtggaaaatggccgcttttctggattca
tcgactgtggccggctgggtgtggcggaccgctatcaggacatagcgttggctacccgtgatattgctgaagagcttggcggcga
atgggctgaccgcttcctcgtgctttacggtatcgccgctcccgattcgcagcgcatcgccttctatcgccttcttgacgagttcttctg
aattattaacgcttacaatttcctgatgcggtattttctccttacgcatctgtgcggtatttcacaccgcatcaggtggcacttttcgggga
aatgtgcgcggaacccctatttgtttatttttctaaatacattcaaatatgtatccgctcatgaccaaaatcccttaacgtgagttttcgttc
cactgagcgtcagaccccgtagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggcttcagcagagcgcagat
accaaatactgttcttctagtgtagccgtagttaggccaccacttcaagaactctgtagcaccgcctacatacctcgctctgctaatcct
gttaccagtggctgctgccagtggcgataagtcgtgtcttaccgggttggactcaagacgatagttaccggataaggcgcagcggt
cgggctgaacgggggggtcgtgcacacagcccagcttggagcgaacgacctacaccgaactgagatacctacagcgtgagcta
tgagaaagcgccacgcttcccgaagggagaaaggcggacaggtatccggtaagcggcagggtcggaacaggagagcgcacg
agggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttc
tttcctgcgttatcccctgattctgtggataaccgtattaccgcctttgagtgagctgataccgctcgccgcagccgaacgaccgagc
gcagcgagtcagtgagcgaggaagcggaagagcgcccaatacgcaaaccgcctctccccgcgcgttggccgattcattaatgc
agctggcacgacaggtttcccgactggaaagcgggcagtgagcgcaacgcaattaatgtgagttagctcactcattaggcacccc
aggctttacactttatgcttccggctcgtatgttgtgtggaattgtgagcggataacaatttcacacaggaaacagctatgaccatgatt
acgccaagcgcgcaattaaccctcactaaagggaacaaaagctggagctgcaagcttggccattgcatacgttgtatccatatcata
atatgtacatttatattggctcatgtccaacattaccgccatgttgacattgattattgactagttattaatagtaatcaattacggggtcatt
agttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgccc
agtacatgaccttatgggactttcctacttggcagtacatctacgtattagtcatcgctattaccatggtgatgcggttttggcagtacat
caatgggcgtggatagcggtttgactcacggggatttccaagtctccaccccattgacgtcaatgggagtttgttttggcaccaaaat
caacgggactttccaaaatgtcgtaacaactccgccccattgacgcaaatgggggtaggcgtgtacggtgggaggtctatataag
cagagctcgtttagtgaaccgGGGTCTCTCTGGTTAGACCAGATCTGAGCCTGGGAGCTCTC
TGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAG
ACCCTTTTAGTCAGTGTGGAAAATCTCTAGCagtggcgcccgaacagggacttgaaagcgaaagg
gaaaccagaggagctctcTCGACGCAGGACTCGGCTTGCTGAAGCGCGCACGGCAAGAG
GCGAGGGGCGGCGACTGGTGAGTACGCCAAAAATTTTGACTAGCGGAGGCTA
GAAGGAGAGAGATGGGTGCGAGAGCGTCAGTATTAAGCGGGGGAGaattagatcgcg
atgggaaaaaattcggttaaggccagggggaaagaaaaaatataaattaaaacatatagtatgggcaagcagggagctagaacg
attcgcagttaatcctggcctgttagaaacatcagaaggctgtagacaaatactgggacagctacaaccatcccttcagacaggatc
agaagaacttagatcattatataatacagtagcaaccctctattgtgtgcatcaaaggatagagataaaagacaccaaggaagcttta
gacaagatagaggaagagcaaaacaaaagtaagaccaccgcacagcaagcGGCCGCTgatcttcagacctggaggagg
agatatgagggacaattggagaagtgaattatataaatataaagtagtaaaaattgaaccattaggagtagcacccaccaaggcaa
agagaagagtggtgcagagagaaaaaagaGCAGTGGGAATaggagctttgttccttgggttcttgggagcagcaggaa
gcactatgggcgcagcgtcaatgacgctgacggtacaggccagacaattattgtctggtatagtgcagcagcagaacaatttgctg
agggctattgaggcgcaacagcatctgttgcaactcacagtctggggcatcaagcagctccaggcaagaatcctggctgtggaaa
gatacctaaaggatcaacagctcctggggatttggggttgctctggaaaactcatttgcaccactgctgtgccttggaatgctagttg
gagtaataaatctctggaacagatttggaatcacacgacctggatggagtgggacagagaaattaacaattacacaagcttaataca
ctccttaattgaagaatcgcaaaaccagcaagaaaagaatgaacaagaattattggaattagataaatgggcaagtttgtggaattg
gtttaacataacaaattggctgtggtatataaaattattcataatgatagtaggaggcttggtaggtttaagaatagtttttgctgtactttc
tatagtgaatagagttaggcagggatattcaccattatcgtttcagacccacctcccaaccccgaggggacccgacaggcccgaa
ggaatagaagaagaaggtggagagagagacagagacagatccattcgattagtgaacggatCTCGACGGTATCGA
TCTCGAcacaaatggcagtattcatccacaattttaaaagaaaaggggggattggggggtacagtgcaggggaaagaatagt
agacataatagcaacagacatacaaactaaagaattacaaaaacaaattacaaaaattcaaaattttcgggtttattacagggacagc
agacatgaggacagctaaaacaataagtaatgtaaaatacagcatagcaaaactttaacctccaaatcaagcctctacttgaatccttt
tctgagggatgaataaggcataggcatcaggggctgttgccaatgtgcattagctgtttgcagcctcaccttctttcatggagtttaag
atatagtgtattttcccaaggtttgaactagctcttcatttctttatgttttaaatgcactgacctcccacattccctttttagtaaaatattcag
aaataatttaaatacatcattgcaatgaaaataaatgttttttattaggcagaatccagatgctcaaggcccttcataatatcccccagttt
agtagttggacttagggaacaaaggaacctttaatagaaattggacagcaagaaagcgagcttagtgatacttgtgggccagggca
ttagccacaccagccaccactttctgataggcagcctgcactggtggggtgaattctttgccaaagtgatgggccagcacacagac
cagcacgttgcccaggagctgtgggaggaagataagaggtatgaacatgattagcaaaagggcctagcttggactcagaataatc
cagccttatcccaaccataaaataaaagcagaatggtagctggattgtagctgctattagcaatatgaaacctcttacatcagttacaa
tttatatgcagaaataccctgttacttctccccttcctatgacatgaacttaaccatagaaaagaaggggaaagaaaacatcaagggt
cccatagactcaccctgaagttctcaggatccacgtgcagcttgtcacagtgcagctcactcagctgggcaaaggtgcccttgagg
ttgtccaggtgagccaggccatcactaaaggcaccgagcactttcttgccatgagccttcaccttagggttgcccataacagcatca
ggagtggacagatccccaaaggactcaaagaacctctgggtccaagggtagaccaccagcagcctaagggtgggaaaataG
GTGAGCATCCTTGTATCCAGCTGGCAGTCCATTCGTCATATCCCATCTGCC
CTGGCCTAGAGCTGGTAAGCATGTGCCGCACTGCGCGATCGAGTGTTGAA
TAActctaccacatggagTTATTCAACACTCGATCGCGCAGCGTGGCACTGCAGGA
GGCCAGGCCAAGAGCTTCTGTGGGGAAGTGAGATCgaccaataggcagagagagtcagt
gcctatcagaaacccaagagtcttctctgtctccacatgcccagtttctattggtctccttaaacctgtcttgtaaccttgataccaacct
gcccagggcctcaccaccaacggcatccacgttcaccttgtcccacagggcagtaacggcagacttctcctcaggagtcaggtgc
accatggtgtctgtttgaggttgctagtgaacacagttgtgtcagaagcaaatgtaagcaatagatggctctgccctgacttttatgcc
cagccctggctcctgccctccctgctcctgggagtagattggccaaccctagggtgtggctccacagggtgaggtctaagtgatga
cagccgtacctgtccttggctcttctggcactggcttaggagttggacttcaaaccctcagccctccctctaagatatatctcttggccc
cataccatcagtacaaattgctactaaaaacatcctcctttgcaagtgtatttaccatcaataattctagccccacaggagtttgttctga
aagtaaacttccacaaccgcaagcttattgaggctaaggcatctgtgaaggaaagaaacatctcctctaaaccactatgctgctaga
gcctcttttctgtactcaagcctcattcagacactagtgtcaccagtctcctcatatacctattgtattttcttcttcttgctggtttagtcatg
ttttctgggagcttaggggcttattttattttgttttgttttctaatcaacagagatgggcaaacccattatttttttctttagacttgggatggt
gatagctgggcagcgtcagaaactgtgtgtggatatagataagagctcaggactatgctgagctgtgatgagggaggggcctagc
taaaggcagtgagagtcagaatgctcctgctattgccttctcagtccccacgcttggtttctacacaagtagatacatagaaaaggct
ataggttagtgtttgagagtcctgcatgattagttgctcagaaatgcccgataaatatgttatgtgtgtttatgtatatatatgttttatatgt
gtgtgtgtgtgtgttgtgtttacaaatatgtgattatcatcaaaacgtgagggTACGTaTATGTGTATATATATAT
ATATATTCAGGAAATAATATATTCTAGAATATGTCACATTCTGTCTCAGGCATC
CATTTTCTTTATGATGCCGTTTGAGGTGGAGTTTTAGTCAGGTGGTCAGCTTCTC
CTTTTTTTTGCCATCTGCCCTGTAAGCATCCTGCTGGGGACCCAGATAGGAGTC
ATCACTCTAGGCTGAGAACATCTGGGCACACACCCTAAGCCTCAGCATGACTC
ATCATGACTCAGCATTGCTGTGCTTGAGCCAGAAGGTTTGCTTAGAAGGTTACA
CAGAACCAGAAGGCGGGGGTGGGGCACTGACCCCGACAGGGGCCTGGCCAGA
ACTGCTCATGCTTGGACTATGGGAGGTCACTAATGGAGACACACAGAAATGTA
ACAGGAACTAAGGAAAAACTGAAGCTTtgggggtataggggagcagtcccatgtagtagtagaatgaa
aaatgctgctatgctgtgcctcccccacctttcccatgtctgccctctactcatggtctatctctcctggctcctgggagtcatggactc
cacccagcaccaccaacctgacctaaccacctatctgagcctgccagcctataacccatctgggccctgatagctggtggccagc
cctgaccccaccccaccctccctggaacctctgatagacacatctggcacaccagctcgcaaagtcaccgtgagggtcttgtgtttg
ctgagtcaaaattccttgaaatccaagtccttagagactcccaggcttggattcaaagctcctgactttctgtctagtgtatgtgcagtg
agccccttttcctctaactgaaagaaggaaaaaaaaatggaacccaaaatattctacatagtttccatgtcacagccagggctgggc
agtctcctgttatttcttttaaaataaatatatcatttaaatgcataaataagcaaaccctgctcgggaatgggagggagagtctctgga
gtccaccccttctcggccctggctctgcagatagtgctatcaaagccctgacagagccctgcccattgctgggccttggagtgagtc
agcctagtagagaggcagggcaagccatctcatagctgctgagtgggagagagaaaagggctcattgtctataaactcaggtcat
ggctattcttattaaaagaaaaggggggactggaagggctaattcactcccaacgaagacaagatctgctttttgcttgtactGGG
TCTCTCTGGTTAGACCAGATCTGAGCCTGGGAGCTCTCTGGCTAACTAGGGAAC
CCACTGCTTAAGCCTCAATAAAGCTTGCCTTGAGTGCTTCAAGTAGTGTGTTGG
TTTTTTGTGTGTGCATTGTCTGAGTAGGTGTCATTCTATTCTGGGGGGTGGGGT
GGGGCAGCACAGCAAGGGGGAGGATTGGGAAGACAATAGCAGGCATGCTGGG
GATGCGGTGGGCTCTATGGagtagtagttcatgtcatcttattattcagtatttataacttgcaaagaaatgaatatca
gagagtgagaggaacttgtttattgcagcttataatggttacaaataaagcaatagcatcacaaatttcacaaataaagcatttttttcac
tgcattctagttgtggtttgtccaaactcatcaatgtatcttatcatgtctggctctagctatcccgcccctaactccgcccATCCCG
CCCCTAACTCCGCCCAGTTCCGCCCATTCTCCGCCCCATGGCTCTCACTACTTCT
GGAATAGCTCAGAGGCCGAGGCGGCCTCGGCCTCTGCATAAATAAAAAAAATT
AGTCGAGGCTTTTTTGGAGGCCTAGGgacgtacccaattcgccctatagtgagtcgtattacgcgcgctca
ctggccgtcgttttacaacgtcgtgactgggaaaaccctggcgttacccaacttaatcgccttgcagcacatccccctttegccagct
ggcgtaatagcgaagaggcccgcaccgatcgcccttcccaacagttgcgcagcctgaatggcgaatggg
XXXXXXXXXX BCL11A shmiR in miR223 (reverse complement)

TABLE 7
Nucleic acid sequence of Mini-G del IVS2 shmiR lentiviral vector (9555 bp) (SEQ
ID NO: 15). shmiR RNA is inserted at the IVS2 deletion (position C in FIG. 1, panel B).

acgcgccctgtagcggcgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttc
gccctttgacgttggagtccacgttctttaatagtggactcttgttccaaactggaacaacactcaaccctatctcggtctattcttttgat
ttataagggattttgccgatttcggcctattggttaaaaaatgagctgatttaacaaaaatttaacgcgaattttaacaaaatattaacgct
tacaatttaggtggcacttttcggggaaatgtgcgcggaacccctatttgtttatttttctaaatacattcaaatatgtatccgctcatgag
acaataaccctgataaatgcttcaataatagcacctagatcaagagacaggatgaggatcgtttcgcatgattgaacaagatggattg
cacgcaggttctccggccgcttgggtggagaggctattcggctatgactgggcacaacagacaatcggctgctctgatgccgccg
tgttccggctgtcagcgcaggggcgcccggttctttttgtcaagaccgacctgtccggtgccctgaatgaactgcaagacgaggca
gcgcggctatcgtggctggccacgacgggcgttccttgcgcagctgtgctcgacgttgtcactgaagcgggaagggactggctg
ctattgggcgaagtgccggggcaggatctcctgtcatctcaccttgctcctgccgagaaagtatccatcatggctgatgcaatgcgg
cggctgcatacgcttgatccggctacctgcccattcgaccaccaagcgaaacatcgcatcgagcgagcacgtactcggatggaag
ccggtcttgtcgatcaggatgatctggacgaagagcatcaggggctcgcgccagccgaactgttcgccaggctcaaggcgagca
tgcccgacggcgaggatctcgtcgtgacccatggcgatgcctgcttgccgaatatcatggtggaaaatggccgcttttctggattca
tcgactgtggccggctgggtgtggcggaccgctatcaggacatagcgttggctacccgtgatattgctgaagagcttggcggcga
atgggctgaccgcttcctcgtgctttacggtatcgccgctcccgattcgcagcgcatcgccttctatcgccttcttgacgagttcttctg
aattattaacgcttacaatttcctgatgcggtattttctccttacgcatctgtgcggtatttcacaccgcatcaggtggcacttttcgggga
aatgtgcgcggaacccctatttgtttatttttctaaatacattcaaatatgtatccgctcatgaccaaaatcccttaacgtgagttttcgttc
cactgagcgtcagaccccgtagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggcttcagcagagcgcagat
accaaatactgttcttctagtgtagccgtagttaggccaccacttcaagaactctgtagcaccgcctacatacctcgctctgctaatcct
gttaccagtggctgctgccagtggcgataagtcgtgtcttaccgggttggactcaagacgatagttaccggataaggcgcagcggt
cgggctgaacgggggggtcgtgcacacagcccagcttggagcgaacgacctacaccgaactgagatacctacagcgtgagcta
tgagaaagcgccacgcttcccgaagggagaaaggcggacaggtatccggtaagcggcagggtcggaacaggagagcgcacg
agggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttc
tttcctgcgttatcccctgattctgtggataaccgtattaccgcctttgagtgagctgataccgctcgccgcagccgaacgaccgagc
gcagcgagtcagtgagcgaggaagcggaagagcgcccaatacgcaaaccgcctctccccgcgcgttggccgattcattaatgc
agctggcacgacaggtttcccgactggaaagcgggcagtgagcgcaacgcaattaatgtgagttagctcactcattaggcacccc
aggctttacactttatgcttccggctcgtatgttgtgtggaattgtgagcggataacaatttcacacaggaaacagctatgaccatgatt
acgccaagcgcgcaattaaccctcactaaagggaacaaaagctggagctgcaagcttggccattgcatacgttgtatccatatcata
atatgtacatttatattggctcatgtccaacattaccgccatgttgacattgattattgactagttattaatagtaatcaattacggggtcatt
agttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgccc
agtacatgaccttatgggactttcctacttggcagtacatctacgtattagtcatcgctattaccatggtgatgcggttttggcagtacat
caatgggcgtggatagcggtttgactcacggggatttccaagtctccaccccattgacgtcaatgggagtttgttttggcaccaaaat
caacgggactttccaaaatgtcgtaacaactccgccccattgacgcaaatgggcggtaggcgtgtacggtgggaggtctatataag
cagagctcgtttagtgaaccgGGGTCTCTCTGGTTAGACCAGATCTGAGCCTGGGAGCTCTC
TGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAG
ACCCTTTTAGTCAGTGTGGAAAATCTCTAGCagtggcgcccgaacagggacttgaaagcgaaagg
gaaaccagaggagctctcTCGACGCAGGACTCGGCTTGCTGAAGCGCGCACGGCAAGAG
GCGAGGGGCGGCGACTGGTGAGTACGCCAAAAATTTTGACTAGCGGAGGCTA
GAAGGAGAGAGATGGGTGCGAGAGCGTCAGTATTAAGCGGGGGAGaattagatcgcg
atgggaaaaaattcggttaaggccagggggaaagaaaaaatataaattaaaacatatagtatgggcaagcagggagctagaacg
attcgcagttaatcctggcctgttagaaacatcagaaggctgtagacaaatactgggacagctacaaccatcccttcagacaggatc
agaagaacttagatcattatataatacagtagcaaccctctattgtgtgcatcaaaggatagagataaaagacaccaaggaagcttta
gacaagatagaggaagagcaaaacaaaagtaagaccaccgcacagcaagcGGCCGCTgatcttcagacctggaggagg
agatatgagggacaattggagaagtgaattatataaatataaagtagtaaaaattgaaccattaggagtagcacccaccaaggcaa
agagaagagtggtgcagagagaaaaaagaGCAGTGGGAATaggagctttgttccttgggttcttgggagcagcaggaa
gcactatgggcgcagcgtcaatgacgctgacggtacaggccagacaattattgtctggtatagtgcagcagcagaacaatttgctg
agggctattgaggcgcaacagcatctgttgcaactcacagtctggggcatcaagcagctccaggcaagaatcctggctgtggaaa
gatacctaaaggatcaacagctcctggggatttggggttgctctggaaaactcatttgcaccactgctgtgccttggaatgctagttg
gagtaataaatctctggaacagatttggaatcacacgacctggatggagtgggacagagaaattaacaattacacaagcttaataca
ctccttaattgaagaatcgcaaaaccagcaagaaaagaatgaacaagaattattggaattagataaatgggcaagtttgtggaattg
gtttaacataacaaattggctgtggtatataaaattattcataatgatagtaggaggcttggtaggtttaagaatagtttttgctgtactttc
tatagtgaatagagttaggcagggatattcaccattatcgtttcagacccacctcccaaccccgaggggacccgacaggcccgaa
ggaatagaagaagaaggtggagagagagacagagacagatccattcgattagtgaacggatCTCGACGGTATCGA
TCTCGAcacaaatggcagtattcatccacaattttaaaagaaaaggggggattggggggtacagtgcaggggaaagaatagt
agacataatagcaacagacatacaaactaaagaattacaaaaacaaattacaaaaattcaaaattttcgggtttattacagggacagc
agacatgaggacagctaaaacaataagtaatgtaaaatacagcatagcaaaactttaacctccaaatcaagcctctacttgaatccttt
tctgagggatgaataaggcataggcatcaggggctgttgccaatgtgcattagctgtttgcagcctcaccttctttcatggagtttaag
atatagtgtattttcccaaggtttgaactagctcttcatttctttatgttttaaatgcactgacctcccacattccctttttagtaaaatattcag
aaataatttaaatacatcattgcaatgaaaataaatgttttttattaggcagaatccagatgctcaaggcccttcataatatcccccagttt
agtagttggacttagggaacaaaggaacctttaatagaaattggacagcaagaaagcgagcttagtgatacttgtgggccagggca
ttagccacaccagccaccactttctgataggcagcctgcactggtggggtgaattctttgccaaagtgatgggccagcacacagac
cagcacgttgcccaggagctgtgggaggaagataagaggtatgaacatgattagcaaaagggcctagcttggactcagaataatc
cagccttatcccaaccataaaataaaagcagaatggtagctggattgtagctgctattagcaatatgaaacctcttacatcagttacaa
tttatatgcagaaataGGTGAGCATCCTTGTATCCAGCTGGCAGTCCATTCGTCATAT
CCCATCTGCCCTGGCCTAGAGCTGGTAAGCATGTGCCGCACTGCGCGATC
GAGTGTTGAATAActctaccacatggagTTATTCAACACTCGATCGCGCAGCGTGG
CACTGCAGGAGGCCAGGCCAAGAGCTTCTGTGGGGAAGTGAGATCccctgttac
ttctccccttcctatgacatgaacttaaccatagaaaagaaggggaaagaaaacatcaagggtcccatagactcaccctgaagttct
caggatccacgtgcagcttgtcacagtgcagctcactcagctgggcaaaggtgcccttgaggttgtccaggtgagccaggccatc
actaaaggcaccgagcactttcttgccatgagccttcaccttagggttgcccataacagcatcaggagtggacagatccccaaagg
actcaaagaacctctgggtccaagggtagaccaccagcagcctaaggggggaaaatagaccaataggcagagagagtcagtg
cctatcagaaacccaagagtcttctctgtctccacatgcccagtttctattggtctccttaaacctgtcttgtaaccttgataccaacctg
cccagggcctcaccaccaacggcatccacgttcaccttgtcccacagggcagtaacggcagacttctcctcaggagtcaggtgca
ccatggtgtctgtttgaggttgctagtgaacacagttgtgtcagaagcaaatgtaagcaatagatggctctgccctgacttttatgccc
agccctggctcctgccctccctgctcctgggagtagattggccaaccctagggtgtggctccacagggtgaggtctaagtgatgac
agccgtacctgtccttggctcttctggcactggcttaggagttggacttcaaaccctcagccctccctctaagatatatctcttggcccc
ataccatcagtacaaattgctactaaaaacatcctcctttgcaagtgtatttaccatcaataattctagccccacaggagtttgttctgaa
agtaaacttccacaaccgcaagcttattgaggctaaggcatctgtgaaggaaagaaacatctcctctaaaccactatgctgctagag
cctcttttctgtactcaagcctcattcagacactagtgtcaccagtctcctcatatacctattgtattttcttcttcttgctggtttagtcatgtt
ttctgggagcttaggggcttattttattttgttttgttttctaatcaacagagatgggcaaacccattatttttttctttagacttgggatggtg
atagctgggcagcgtcagaaactgtgtgtggatatagataagagctcaggactatgctgagctgtgatgagggaggggcctagct
aaaggcagtgagagtcagaatgctcctgctattgccttctcagtccccacgcttggtttctacacaagtagatacatagaaaaggcta
taggttagtgtttgagagtcctgcatgattagttgctcagaaatgcccgataaatatgttatgtgtgtttatgtatatatatgttttatatgtgt
gtgtgtgtgtgttgtgtttacaaatatgtgattatcatcaaaacgtgagggTACGTaTATGTGTATATATATATA
TATATTCAGGAAATAATATATTCTAGAATATGTCACATTCTGTCTCAGGCATCC
ATTTTCTTTATGATGCCGTTTGAGGTGGAGTTTTAGTCAGGTGGTCAGCTTCTCC
TTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCA
TCATGACTCAGCATTGCTGTGCTTGAGCCAGAAGGTTTGCTTAGAAGGTTACAC
AGAACCAGAAGGCGGGGGTGGGGCACTGACCCCGACAGGGGCCTGGCCAGAA
CTGCTCATGCTTGGACTATGGGAGGTCACTAATGGAGACACACAGAAATGTAA
CAGGAACTAAGGAAAAACTGAAGCTTtgggggtataggggagcagtcccatgtagtagtagaatgaaaa
atgctgctatgctgtgcctcccccacctttcccatgtctgccctctactcatggtctatctctcctggctcctgggagtcatggactcca
cccagcaccaccaacctgacctaaccacctatctgagcctgccagcctataacccatctgggccctgatagctggtggccagccct
gaccccaccccaccctccctggaacctctgatagacacatctggcacaccagctcgcaaagtcaccgtgagggtcttgtgtttgct
gagtcaaaattccttgaaatccaagtccttagagactcccaggcttggattcaaagctcctgactttctgtctagtgtatgtgcagtgag
ccccttttcctctaactgaaagaaggaaaaaaaaatggaacccaaaatattctacatagtttccatgtcacagccagggctgggcagt
ctcctgttatttcttttaaaataaatatatcatttaaatgcataaataagcaaaccctgctcgggaatgggagggagagtctctggagtc
caccccttctcggccctggctctgcagatagtgctatcaaagccctgacagagccctgcccattgctgggccttggagtgagtcag
cctagtagagaggcagggcaagccatctcatagctgctgagtgggagagagaaaagggctcattgtctataaactcaggtcatgg
ctattcttattaaaagaaaaggggggactggaagggctaattcactcccaacgaagacaagatctgctttttgcttgtactGGGTC
TCTCTGGTTAGACCAGATCTGAGCCTGGGAGCTCTCTGGCTAACTAGGGAACC
CACTGCTTAAGCCTCAATAAAGCTTGCCTTGAGTGCTTCAAGTAGTGTGTTGGT
TTTTTGTGTGTGCATTGTCTGAGTAGGTGTCATTCTATTCTGGGGGGTGGGGTG
GGGCAGCACAGCAAGGGGGAGGATTGGGAAGACAATAGCAGGCATGCTGGGG
ATGCGGTGGGCTCTATGGagtagtagttcatgtcatcttattattcagtatttataacttgcaaagaaatgaatatcaga
gagtgagaggaacttgtttattgcagcttataatggttacaaataaagcaatagcatcacaaatttcacaaataaagcatttttttcactg
cattctagttgtggtttgtccaaactcatcaatgtatcttatcatgtctggctctagctatcccgcccctaactccgcccATCCCGC
CCCTAACTCCGCCCAGTTCCGCCCATTCTCCGCCCCATGGCTCTCACTACTTCTG
GAATAGCTCAGAGGCCGAGGCGGCCTCGGCCTCTGCATAAATAAAAAAAATTA
GTCGAGGCTTTTTTGGAGGCCTAGGgacgtacccaattcgccctatagtgagtcgtattacgcgegctcact
ggccgtcgttttacaacgtcgtgactgggaaaaccctggcgttacccaacttaatcgccttgcagcacatccccctttcgccagctg
gcgtaatagcgaagaggcccgcaccgatcgcccttcccaacagttgcgcagcctgaatggcgaatggg

| | |
|---|---|
| XXXXXXXXXX | BCL11A shmiR in miR223 (reverse complement) |

TABLE 8
Nucleic acid sequence of Mini-G 3′UTR shmiR lentiviral vector (SEQ ID NO:16).
shmiR RNA is inserted at the IVS1 end position (position B in FIG. 1, panel B).
acgcgccctgtagcggcgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttc
gccctttgacgttggagtccacgttctttaatagtggactcttgttccaaactggaacaacactcaaccctatctcggtctattcttttgat
ttataagggattttgccgatttcggcctattggttaaaaaatgagctgatttaacaaaaatttaacgcgaattttaacaaaatattaacgct
tacaatttaggtggcacttttcggggaaatgtgcgcggaacccctatttgtttatttttctaaatacattcaaatatgtatccgctcatgag
acaataaccctgataaatgcttcaataatagcacctagatcaagagacaggatgaggatcgtttcgcatgattgaacaagatggattg
cacgcaggttctccggccgcttgggtggagaggctattcggctatgactgggcacaacagacaatcggctgctctgatgccgccg
tgttccggctgtcagcgcaggggcgcccggttctttttgtcaagaccgacctgtccggtgccctgaatgaactgcaagacgaggca
gcgcggctatcgtggctggccacgacgggcgttccttgcgcagctgtgctcgacgttgtcactgaagcgggaagggactggctg
ctattgggcgaagtgccggggcaggatctcctgtcatctcaccttgctcctgccgagaaagtatccatcatggctgatgcaatgcgg
cggctgcatacgcttgatccggctacctgcccattcgaccaccaagcgaaacatcgcatcgagcgagcacgtactcggatggaag
ccggtcttgtcgatcaggatgatctggacgaagagcatcaggggctcgcgccagccgaactgttcgccaggctcaaggcgagca
tgcccgacggcgaggatctcgtcgtgacccatggcgatgcctgcttgccgaatatcatggtggaaaatggccgcttttctggattca
tcgactgtggccggctgggtgtggcggaccgctatcaggacatagcgttggctacccgtgatattgctgaagagcttggcggcga
atgggctgaccgcttcctcgtgctttacggtatcgccgctcccgattcgcagcgcatcgccttctatcgccttcttgacgagttcttctg
aattattaacgcttacaatttcctgatgcggtattttctccttacgcatctgtgcggtatttcacaccgcatcaggtggcacttttcgggga
aatgtgcgcggaacccctatttgtttatttttctaaatacattcaaatatgtatccgctcatgaccaaaatcccttaacgtgagttttcgttc
cactgagcgtcagaccccgtagaaaagatcaaaggatcttcttgagatcctttttttctgcgcgtaatctgctgcttgcaaacaaaaaa
accaccgctaccagcggtggtttgtttgccggatcaagagctaccaactctttttccgaaggtaactggcttcagcagagcgcagat
accaaatactgttcttctagtgtagccgtagttaggccaccacttcaagaactctgtagcaccgcctacatacctcgctctgctaatcct
gttaccagtggctgctgccagtggcgataagtcgtgtcttaccgggttggactcaagacgatagttaccggataaggcgcagcggt
cgggctgaacgggggggtcgtgcacacagcccagcttggagcgaacgacctacaccgaactgagatacctacagcgtgagcta
tgagaaagcgccacgcttcccgaagggagaaaggcggacaggtatccggtaagcggcagggtcggaacaggagagcgcacg
agggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttc
tttcctgcgttatcccctgattctgtggataaccgtattaccgcctttgagtgagctgataccgctcgccgcagccgaacgaccgagc
gcagcgagtcagtgagcgaggaagcggaagagcgcccaatacgcaaaccgcctctccccgcgcgttggccgattcattaatgc
agctggcacgacaggtttcccgactggaaagcgggcagtgagcgcaacgcaattaatgtgagttagctcactcattaggcacccc
aggctttacactttatgcttccggctcgtatgttgtgtggaattgtgagcggataacaatttcacacaggaaacagctatgaccatgatt
acgccaagcgcgcaattaaccctcactaaagggaacaaaagctggagctgcaagcttggccattgcatacgttgtatccatatcata
atatgtacatttatattggctcatgtccaacattaccgccatgttgacattgattattgactagttattaatagtaatcaattacggggtcatt
agttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgccc
agtacatgaccttatgggactttcctacttggcagtacatctacgtattagtcatcgctattaccatggtgatgcggttttggcagtacat
caatgggcgtggatagcggtttgactcacggggatttccaagtctccaccccattgacgtcaatgggagtttgttttggcaccaaaat
caacgggactttccaaaatgtcgtaacaactccgccccattgacgcaaatgggcggtaggcgtgtacggtgggaggtctatataag
cagagctcgtttagtgaaccgGGGTCTCTCTGGTTAGACCAGATCTGAGCCTGGGAGCTCTC
TGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAG
ACCCTTTTAGTCAGTGTGGAAAATCTCTAGCagtggcgcccgaacagggacttgaaagcgaaagg
gaaaccagaggagctctcTCGACGCAGGACTCGGCTTGCTGAAGCGCGCACGGCAAGAG
GCGAGGGGCGGCGACTGGTGAGTACGCCAAAAATTTTGACTAGCGGAGGCTA
GAAGGAGAGAGATGGGTGCGAGAGCGTCAGTATTAAGCGGGGGAGaattagatcgcg
atgggaaaaaattcggttaaggccagggggaaagaaaaaatataaattaaaacatatagtatgggcaagcagggagctagaacg
attcgcagttaatcctggcctgttagaaacatcagaaggctgtagacaaatactgggacagctacaaccatcccttcagacaggatc
agaagaacttagatcattatataatacagtagcaaccctctattgtgtgcatcaaaggatagagataaaagacaccaaggaagcttta
gacaagatagaggaagagcaaaacaaaagtaagaccaccgcacagcaagcGGCCGCTgatcttcagacctggaggagg
agatatgagggacaattggagaagtgaattatataaatataaagtagtaaaaattgaaccattaggagtagcacccaccaaggcaa
agagaagagtggtgcagagagaaaaaagaGCAGTGGGAATaggagctttgttccttgggttcttgggagcagcaggaa
gcactatgggcgcagcgtcaatgacgctgacggtacaggccagacaattattgtctggtatagtgcagcagcagaacaatttgctg
agggctattgaggcgcaacagcatctgttgcaactcacagtctggggcatcaagcagctccaggcaagaatcctggctgtggaaa
gatacctaaaggatcaacagctcctggggatttggggttgctctggaaaactcatttgcaccactgctgtgccttggaatgctagttg
gagtaataaatctctggaacagatttggaatcacacgacctggatggagtgggacagagaaattaacaattacacaagcttaataca
ctccttaattgaagaatcgcaaaaccagcaagaaaagaatgaacaagaattattggaattagataaatgggcaagtttgtggaattg
gtttaacataacaaattggctgtggtatataaaattattcataatgatagtaggaggcttggtaggtttaagaatagtttttgctgtactttc
tatagtgaatagagttaggcagggatattcaccattatcgtttcagacccacctcccaaccccgaggggacccgacaggcccgaa
ggaatagaagaagaaggtggagagagagacagagacagatccattcgattagtgaacggatCTCGACGGTATCGA
TCTCGAcacaaatggcagtattcatccacaattttaaaagaaaaggggggattggggggtacagtgcaggggaaagaatagt
agacataatagcaacagacatacaaactaaagaattacaaaaacaaattacaaaaattcaaaattttcgggtttattacagggacagc
agacatgaggacagctaaaacaataagtaatgtaaaatacagcatagcaaaactttaacctccaaatcaagcctctacttgaatccttt
tctgagggatgaataaggcataggcatcaggggctgttgccaatgtgcattagctgtttgcagcctcaccttctttcatggagtttaag
atatagtgtattttcccaaggtttgaactagctcttcatttctttatgttttaaatgcactgacctcccacattccctttttagtaaaatattcag
aaataatttaaatacatcattgcaatgaaaataaatgttttttattaggcagaatccagatgctcaaggcccttcataatatGGTGA
GCATCCTTGTATCCAGCTGGCAGTCCATTCGTCATATCCCATCTGCCCTGG
CCTAGAGCTGGTAAGCATGTGCCGCACTGCGCGATCGAGTGTTGAATAAct
ctaccacatggagTTATTCAACACTCGATCGCGCAGCGTGGCACTGCAGGAGGCC
AGGCCAAGAGCTTCTGTGGGGAAGTGAGATCcccccagtttagtagttggacttagggaacaaa
ggaacctttaatagaaattggacagcaagaaagcgagcttagtgatacttgtgggccagggcattagccacaccagccaccacttt
ctgataggcagcctgcactggtggggtgaattctttgccaaagtgatgggccagcacacagaccagcacgttgcccaggagctgt
gggaggaagataagaggtatgaacatgattagcaaaagggcctagcttggactcagaataatccagccttatcccaaccataaaat
aaaagcagaatggtagctggattgtagctgctattagcaatatgaaacctcttacatcagttacaatttatatgcagaaataccctgtta
cttctccccttcctatgacatgaacttaaccatagaaaagaaggggaaagaaaacatcaagggtcccatagactcaccctgaagttc
tcaggatccacgtgcagcttgtcacagtgcagctcactcagctgggcaaaggtgcccttgaggttgtccaggtgagccaggccatc
actaaaggcaccgagcactttcttgccatgagccttcaccttagggttgcccataacagcatcaggagtggacagatccccaaagg
actcaaagaacctctgggtccaagggtagaccaccagcagcctaaggggggaaaatagaccaataggcagagagagtcagtg
cctatcagaaacccaagagtcttctctgtctccacatgcccagtttctattggtctccttaaacctgtcttgtaaccttgataccaacctg
cccagggcctcaccaccaacggcatccacgttcaccttgtcccacagggcagtaacggcagacttctcctcaggagtcaggtgca
ccatggtgtctgtttgaggttgctagtgaacacagttgtgtcagaagcaaatgtaagcaatagatggctctgccctgacttttatgccc
agccctggctcctgccctccctgctcctgggagtagattggccaaccctagggtgtggctccacagggtgaggtctaagtgatgac
agccgtacctgtccttggctcttctggcactggcttaggagttggacttcaaaccctcagccctccctctaagatatatctcttggcccc
ataccatcagtacaaattgctactaaaaacatcctcctttgcaagtgtatttaccatcaataattctagccccacaggagtttgttctgaa
agtaaacttccacaaccgcaagcttattgaggctaaggcatctgtgaaggaaagaaacatctcctctaaaccactatgctgctagag
cctcttttctgtactcaagcctcattcagacactagtgtcaccagtctcctcatatacctattgtattttcttcttcttgctggtttagtcatgtt
ttctgggagcttaggggcttattttattttgttttgttttctaatcaacagagatgggcaaacccattatttttttctttagacttgggatggtg
atagctgggcagcgtcagaaactgtgtgtggatatagataagagctcaggactatgctgagctgtgatgagggaggggcctagct
aaaggcagtgagagtcagaatgctcctgctattgccttctcagtccccacgcttggtttctacacaagtagatacatagaaaaggcta
taggttagtgtttgagagtcctgcatgattagttgctcagaaatgcccgataaatatgttatgtgtgtttatgtatatatatgttttatatgtgt
gtgtgtgtgtgttgtgtttacaaatatgtgattatcatcaaaacgtgagggTACGTaTATGTGTATATATATATA
TATATTCAGGAAATAATATATTCTAGAATATGTCACATTCTGTCTCAGGCATCC
ATTTTCTTTATGATGCCGTTTGAGGTGGAGTTTTAGTCAGGTGGTCAGCTTCTCC
TTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCA
TCATGACTCAGCATTGCTGTGCTTGAGCCAGAAGGTTTGCTTAGAAGGTTACAC
AGAACCAGAAGGCGGGGGTGGGGCACTGACCCCGACAGGGGCCTGGCCAGAA
CTGCTCATGCTTGGACTATGGGAGGTCACTAATGGAGACACACAGAAATGTAA
CAGGAACTAAGGAAAAACTGAAGCTTtgggggtataggggagcagtcccatgtagtagtagaatgaaaa
atgctgctatgctgtgcctcccccacctttcccatgtctgccctctactcatggtctatctctcctggctcctgggagtcatggactcca
cccagcaccaccaacctgacctaaccacctatctgagcctgccagcctataacccatctgggccctgatagctggtggccagccct
gaccccaccccaccctccctggaacctctgatagacacatctggcacaccagctcgcaaagtcaccgtgagggtcttgtgtttgct
gagtcaaaattccttgaaatccaagtccttagagactcccaggcttggattcaaagctcctgactttctgtctagtgtatgtgcagtgag
ccccttttcctctaactgaaagaaggaaaaaaaaatggaacccaaaatattctacatagtttccatgtcacagccagggctgggcagt
ctcctgttatttcttttaaaataaatatatcatttaaatgcataaataagcaaaccctgctcgggaatgggagggagagtctctggagtc
caccccttctcggccctggctctgcagatagtgctatcaaagccctgacagagccctgcccattgctgggccttggagtgagtcag
cctagtagagaggcagggcaagccatctcatagctgctgagtgggagagagaaaagggctcattgtctataaactcaggtcatgg
ctattcttattaaaagaaaaggggggactggaagggctaattcactcccaacgaagacaagatctgctttttgcttgtactGGGTC
TCTCTGGTTAGACCAGATCTGAGCCTGGGAGCTCTCTGGCTAACTAGGGAACC
CACTGCTTAAGCCTCAATAAAGCTTGCCTTGAGTGCTTCAAGTAGTGTGTTGGT
TTTTTGTGTGTGCATTGTCTGAGTAGGTGTCATTCTATTCTGGGGGGTGGGGTG
GGGCAGCACAGCAAGGGGGAGGATTGGGAAGACAATAGCAGGCATGCTGGGG
ATGCGGTGGGCTCTATGGagtagtagttcatgtcatcttattattcagtatttataacttgcaaagaaatgaatatcaga
gagtgagaggaacttgtttattgcagcttataatggttacaaataaagcaatagcatcacaaatttcacaaataaagcatttttttcactg
cattctagttgtggtttgtccaaactcatcaatgtatcttatcatgtctggctctagctatcccgcccctaactccgcccATCCCGC
CCCTAACTCCGCCCAGTTCCGCCCATTCTCCGCCCCATGGCTCTCACTACTTCTG
GAATAGCTCAGAGGCCGAGGCGGCCTCGGCCTCTGCATAAATAAAAAAAATTA
GTCGAGGCTTTTTTGGAGGCCTAGGgacgtacccaattcgccctatagtgagtcgtattacgcgcgctcact
ggccgtcgttttacaacgtcgtgactgggaaaaccctggcgttacccaacttaatcgccttgcagcacatccccctttcgccagctg
gcgtaatagcgaagaggcccgcaccgatcgcccttcccaacagttgcgcagcctgaatggcgaatggg
XXXXXXXXXX BCL11A shmiR in miR223 (reverse complement)

### Conclusions

In view of the foregoing, it was observed that incorporation of shRNA^{miR} does not significantly lower titer in comparison to UV+EC1. UV+EC1 shRNA^{miR} vectors are capable of maintaining % β^{AS3}/VCN expression while increasing fetal globin expression by 10-15 fold. The UV+EC1 shRNA^{miR} vectors show therapeutic potential for treating sickle cell disease and UV+EC1 with the shRNA^{miR} in the End IVS2 position was selected as the lead candidate vector.

### Example 2

### Evaluation of LV Comprising βAS3, BCL11A shmiR in miR223 and ZNF410 shmiR in miR144

In this experiment, two LV constructs were evaluated: 1) Mini-G end IVS2 shmiR BCL11A-ZNF410 (see, FIG. 4, panel A, Table 9, SEQ ID NO: 17) and 2) Mini-G del IVS2 shmiR BCL11A-ZNF410 (see, FIG. 4, panel B, Table 10, SEQ ID NO: 18).

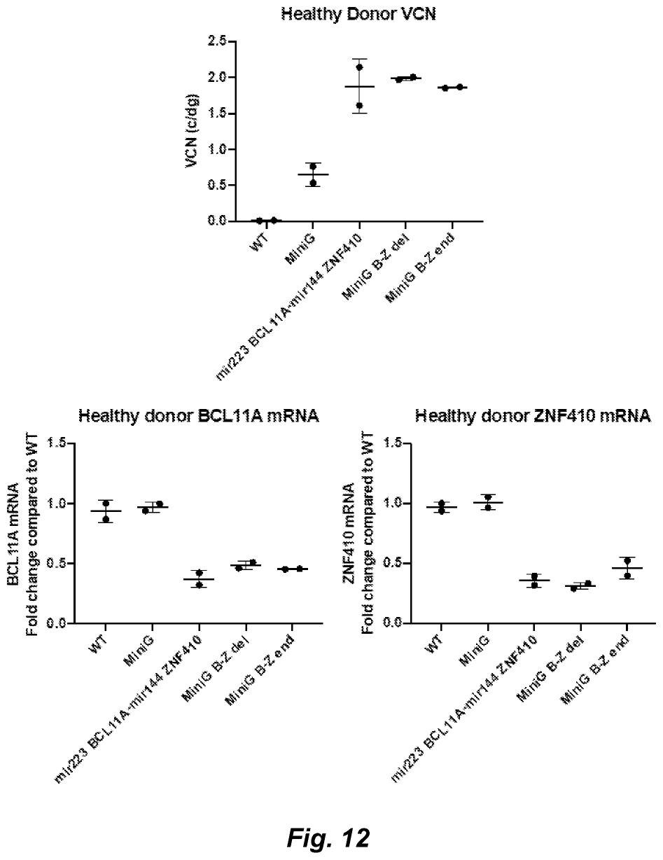

FIG. 12, top, illustrates the vector copy number (VCN) in health donor cells transduced with a DS shmiR vector containing mir223BCL11A-mir144ZNF410 and lacking βAS3, the MiniG-BCL11A-ZNF410-IVS2 del vector containing βAS3 (FIG. 4, panel A (SEQ ID NO:18) (aka miniG B-Z del) and the MiniG-BCL11A-ZNF410-IVS2 end vector containing βAS3 (FIG. 4, panel B, SEQ ID NO: 17) (aka miniG B-Z end). The lower panel shows expression levels of BCL11 and ZNF410 mRNAs. As shown therein, VCN was significant and there was also significant reduction in of BCL11 and ZNF410 mRNAs.

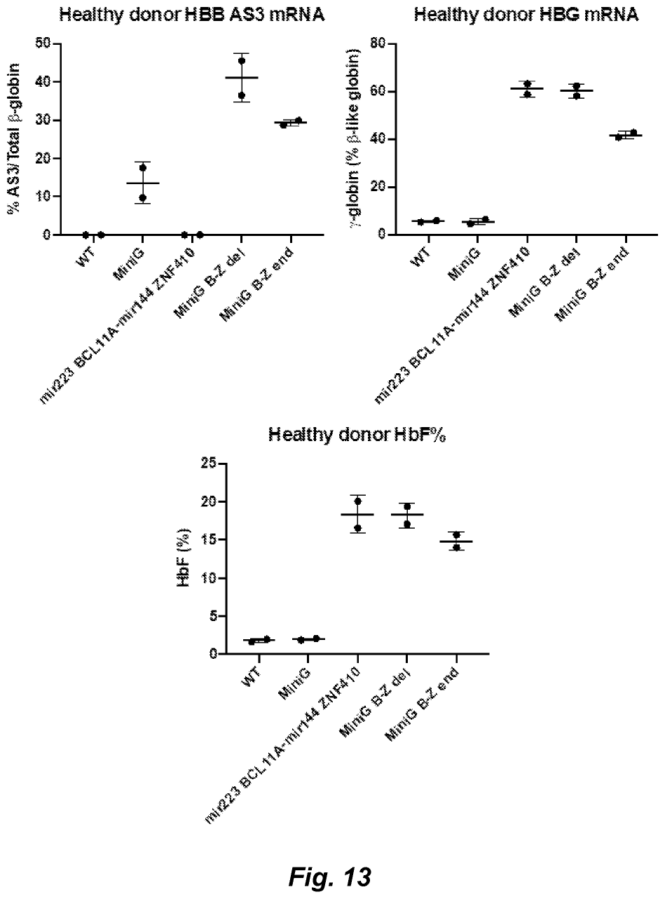

FIG. 13 shows levels of HBB βAS3 mRNA, healthy donor hemoglobin mRNA, and healthy donor fetal hemoglobin (HbF) mRNA in the same cells referenced in FIG. 12. As shown therein, MiniG B-Z del and MiniG B-Z transduced cells produced significant levels of βAS3 and HBG mRNA. All transduced cells produced significant levels of HBG and HbF mRNA.

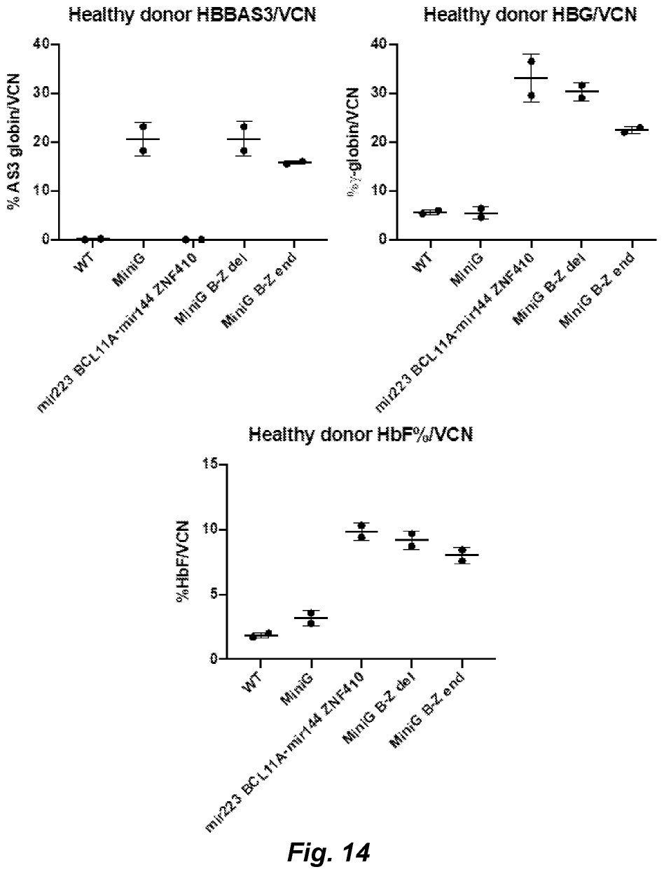

FIG. 14 shows levels of HBB βAS3 mRNA, healthy donor hemoglobin mRNA, and healthy donor fetal hemoglobin (HbF) mRNA normalized to vector copy number (VCN). As shown therein, MiniG B-Z del and MiniG B-Z transduced cells produced significant levels of βAS3 and HBG mRNA. All transduced cells produced significant levels of HBG and HbF mRNA.

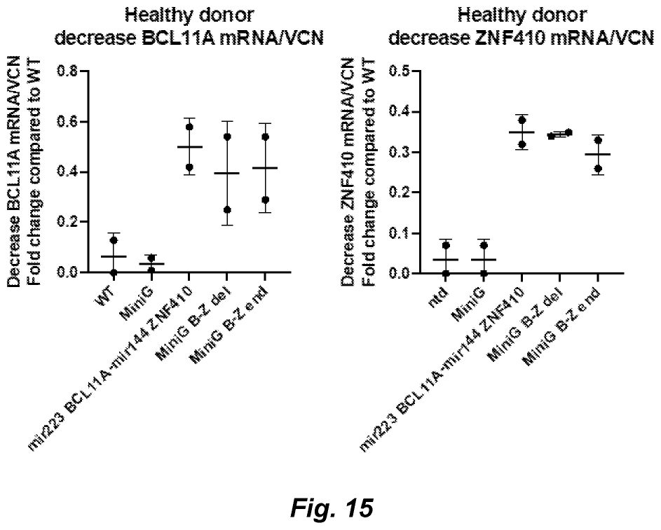

FIG. 15 shows the decrease in BCL11A mRNA and ZNF410 mRNA normalized to vector copy number. As shown therein, all three vectors significantly decreased BCL11A and ZNF410 mRNA.

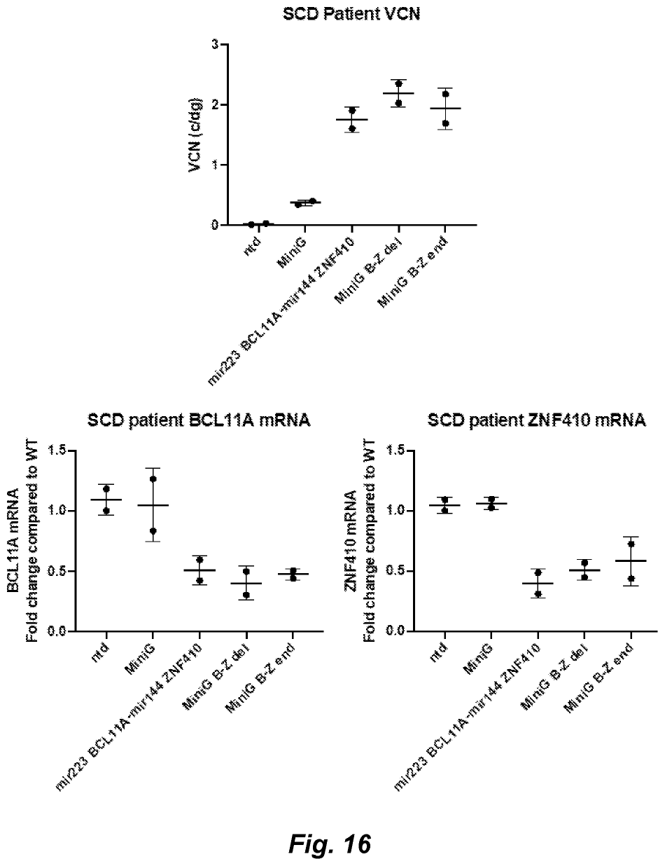

FIG. 16 top, illustrates the vector copy number (VCN) in cells obtained from a sickle cell disease (SCD) patient transduced with a DS shmiR vector containing mir223BCL11A-mir144ZNF410 and lacking βAS3, the MiniG-BCL11A-ZNF410-IVS2 del vector containing βAS3 (FIG. 4, panel A (SEQ ID NO:18) (aka miniG B-Z del) and the MiniG-BCL11A-ZNF410-IVS2 end vector containing βAS3 (FIG. 4, panel B, SEQ ID NO: 17) (aka miniG B-Z end). The lower panel shows expression levels of BCL11 and ZNF410 mRNAs. As shown therein, VCN was significant and there was also significant reduction in of BCL11 and ZNF410 mRNAs.

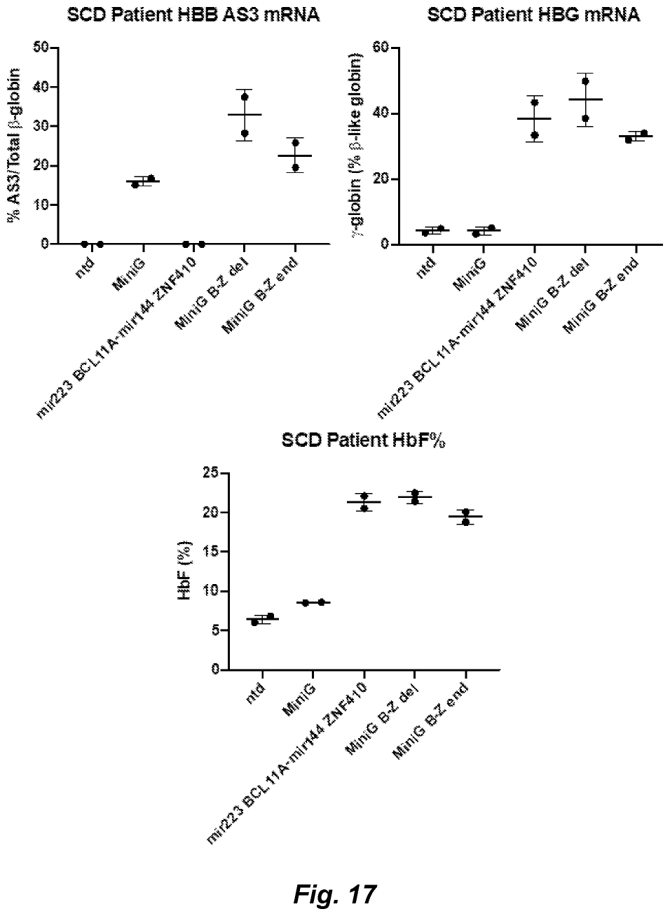

FIG. 17 shows levels of SCD patient HBB βAS3 mRNA, SCD patient hemoglobin mRNA, and SCD patient fetal hemoglobin (HbF) mRNA in the same cells referenced in FIG. 16. As shown therein, MiniG B-Z del and MiniG B-Z transduced cells produced significant levels of βAS3 and HBG mRNA. All transduced cells produced significant levels of HBG and HbF mRNA.

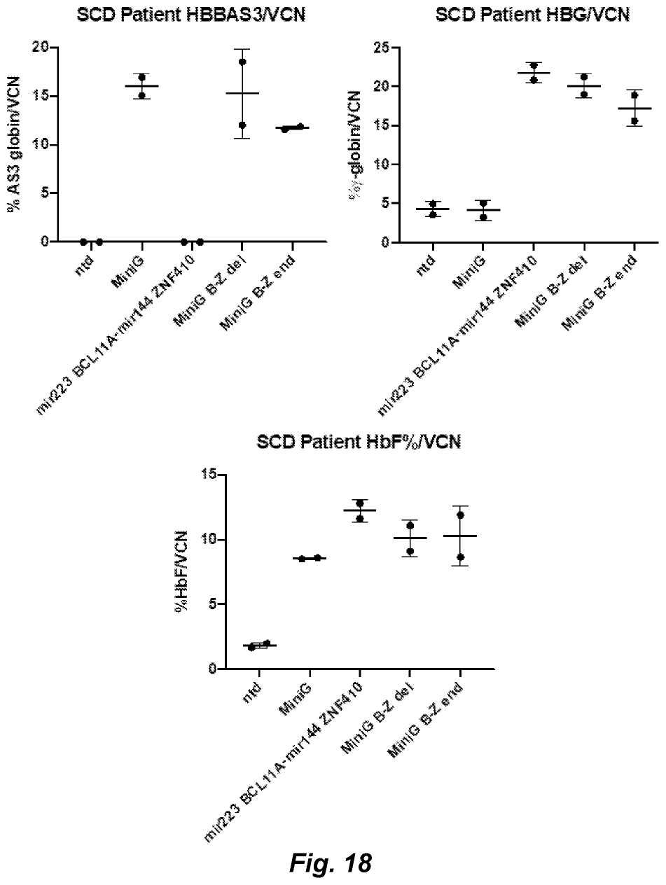

FIG. 18 shows levels of HBB βAS3 mRNA in SCD patient cells, SCD patient hemoglobin mRNA, and SCD patient fetal hemoglobin (HbF) mRNA normalized to vector copy number (VCN). As shown therein, MiniG B-Z del and MiniG B-Z transduced cells produced significant levels of βAS3 and HBG mRNA. All transduced cells produced significant levels of HBG and HbF mRNA.

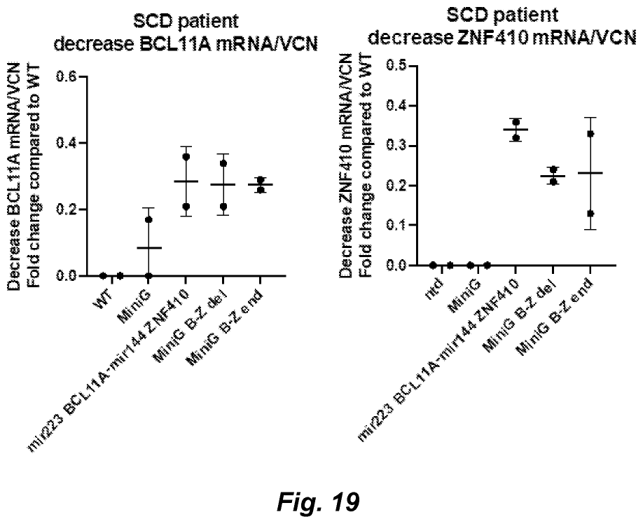

FIG. 19 shows the decrease in BCL11A mRNA and ZNF410 mRNA in SCD patient cells normalized to vector copy number. As shown therein, all three vectors significantly decreased BCL11A and ZNF410 mRNA.

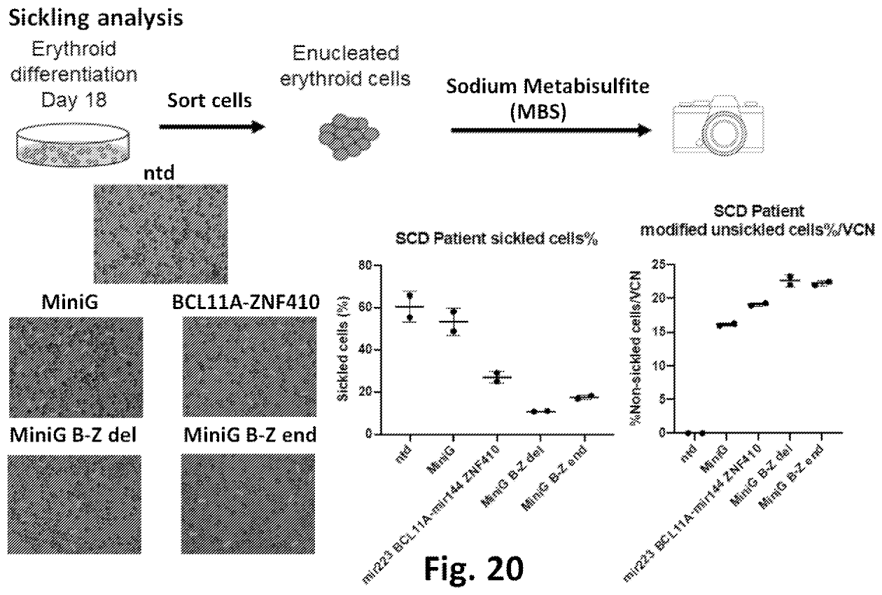

FIG. 20 illustrates an analysis of sodium metabisulfite induced sickling in cells from an SCD patient transduced with the BCL11A-ZNF410, MiniG B-Z del, and MiniG B-Z end vectors. As shown therein, all three vectors reduced sickling, with the MiniG B-Z del, and MiniG B-Z end vectors proving most effective in reducing sodium metabisulfite induced sickling.

In another experiment, MiniG, BCL11A shmiR, MiniG-BCL11A, BCL11A-Zfp410, MiniG-BCL11A-Zfp410 vectors were compared. Analyses similar to those described above were performed yielding similar results

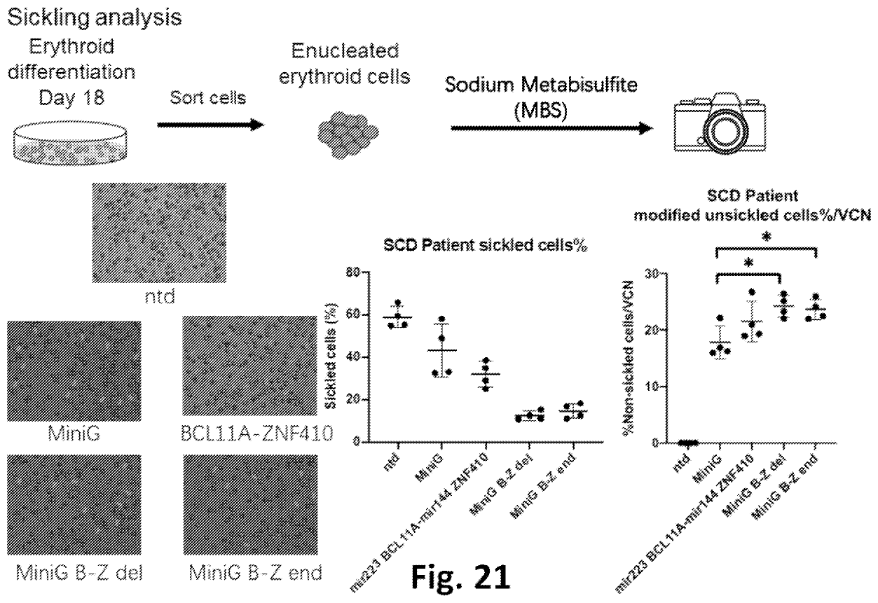

FIG. 21 illustrates an analysis of sodium metabisulfite induced sickling in cells from an SCD patient transduced with the MiniG vector, mir223 BCL11a-mir144ZNF410, MiniG B-Z del, and MiniG B-Z end vectors. As shown therein, the MiniG B-Z del, and MiniG B-Z end vectors produced a significant reduction in sickling.

TABLE 9
Mini-G end IVS2 shmiR BCL11A-ZNF410 (SEQ ID NO: 17)
(see, e.g., FIG. 4, panel A.

| | |
|---|---|
| ACGCGCCCTGTAGCGGCGCATTAAGCGCGGCGGGTGTGGTGGTTAC | CMV |
| GCGCAGCGTGACCGCTACACTTGCCAGCGCCCTAGCGCCCGCTCCTT | R |
| TCGCTTTCTTCCCTTCCTTTCTCGCCACGTTCGCCGGCTTTCCCCGTC | U5 |
| AAGCTCTAAATCGGGGGCTCCCTTTAGGGTTCCGATTTAGTGCTTTA | RRE |
| CGGCACCTCGACCCCAAAAAACTTGATTAGGGTGATGGTTCACGTA | CPPT |
| GTGGGCCATCGCCCTGATAGACGGTTTTTCGCCCTTTGACGTTGGAG | 3′UTR |
| TCCACGTTCTTTAATAGTGGACTCTTGTTCCAAACTGGAACAACACT | ZNF410 |
| CAACCCTATCTCGGTCTATTCTTTTGATTTATAAGGGATTTTGCCGAT | shmiR in |
| TTCGGCCTATTGGTTAAAAAATGAGCTGATTTAACAAAAATTTAACG | miR144 |
| CGAATTTTAACAAAATATTAACGCTTACAATTTAGGTGGCACTTTTC | Rev |
| GGGGAAATGTGCGCGGAACCCCTATTTGTTTATTTTTCTAAATACAT | comp |
| TCAAATATGTATCCGCTCATGAGACAATAACCCTGATAAATGCTTCA | BCL11A |
| ATAATAGCACCTAGATCAAGAGACAGGATGAGGATCGTTTCGCATG | shmiR in |
| ATTGAACAAGATGGATTGCACGCAGGTTCTCCGGCCGCTTGGGTGG | miR223 |
| AGAGGCTATTCGGCTATGACTGGGCACAACAGACAATCGGCTGCTC | Rev |
| TGATGCCGCCGTGTTCCGGCTGTCAGCGCAGGGGCGCCCGGTTCTTT | comp |
| TTGTCAAGACCGACCTGTCCGGTGCCCTGAATGAACTGCAAGACGA | Globin- |
| GGCAGCGCGGCTATCGTGGCTGGCCACGACGGGCGTTCCTTGCGCA | promoter |
| GCTGTGCTCGACGTTGTCACTGAAGCGGGAAGGGACTGGCTGCTATT | LCR |
| GGGCGAAGTGCCGGGGCAGGATCTCCTGTCATCTCACCTTGCTCCTG | Delta U3 |
| CCGAGAAAGTATCCATCATGGCTGATGCAATGCGGCGGCTGCATAC | R |

GCTTGATCCGGCTACCTGCCCATTCGACCACCAAGCGAAACATCGCA
TCGAGCGAGCACGTACTCGGATGGAAGCCGGTCTTGTCGATCAGGA
TGATCTGGACGAAGAGCATCAGGGGCTCGCGCCAGCCGAACTGTTC
GCCAGGCTCAAGGCGAGCATGCCCGACGGCGAGGATCTCGTCGTGA
CCCATGGCGATGCCTGCTTGCCGAATATCATGGTGGAAAATGGCCG
CTTTTCTGGATTCATCGACTGTGGCCGGCTGGGTGTGGCGGACCGCT
ATCAGGACATAGCGTTGGCTACCCGTGATATTGCTGAAGAGCTTGGC
GGCGAATGGGCTGACCGCTTCCTCGTGCTTTACGGTATCGCCGCTCC
CGATTCGCAGCGCATCGCCTTCTATCGCCTTCTTGACGAGTTCTTCTG
AATTATTAACGCTTACAATTTCCTGATGCGGTATTTTCTCCTTACGCA
TCTGTGCGGTATTTCACACCGCATCAGGTGGCACTTTTCGGGGAAAT
GTGCGCGGAACCCCTATTTGTTTATTTTTCTAAATACATTCAAATATG
TATCCGCTCATGACCAAAATCCCTTAACGTGAGTTTTCGTTCCACTG
AGCGTCAGACCCCGTAGAAAAGATCAAAGGATCTTCTTGAGATCCT
TTTTTTCTGCGCGTAATCTGCTGCTTGCAAACAAAAAAACCACCGCT
ACCAGCGGTGGTTTGTTTGCCGGATCAAGAGCTACCAACTCTTTTTC
CGAAGGTAACTGGCTTCAGCAGAGCGCAGATACCAAATACTGTTCT
TCTAGTGTAGCCGTAGTTAGGCCACCACTTCAAGAACTCTGTAGCAC
CGCCTACATACCTCGCTCTGCTAATCCTGTTACCAGTGGCTGCTGCC
AGTGGCGATAAGTCGTGTCTTACCGGGTTGGACTCAAGACGATAGTT
ACCGGATAAGGCGCAGCGGTCGGGCTGAACGGGGGGGTCGTGCACA
CAGCCCAGCTTGGAGCGAACGACCTACACCGAACTGAGATACCTAC
AGCGTGAGCTATGAGAAAGCGCCACGCTTCCCGAAGGGAGAAAGGC
GGACAGGTATCCGGTAAGCGGCAGGGTCGGAACAGGAGAGCGCAC
GAGGGAGCTTCCAGGGGGAAACGCCTGGTATCTTTATAGTCCTGTCG
GGTTTCGCCACCTCTGACTTGAGCGTCGATTTTTGTGATGCTCGTCA
GGGGGGCGGAGCCTATGGAAAAACGCCAGCAACGCGGCCTTTTTAC
GGTTCCTGGCCTTTTGCTGGCCTTTTGCTCACATGTTCTTTCCTGCGT
TATCCCCTGATTCTGTGGATAACCGTATTACCGCCTTTGAGTGAGCT
GATACCGCTCGCCGCAGCCGAACGACCGAGCGCAGCGAGTCAGTGA
GCGAGGAAGCGGAAGAGCGCCCAATACGCAAACCGCCTCTCCCCGC
GCGTTGGCCGATTCATTAATGCAGCTGGCACGACAGGTTTCCCGACT
GGAAAGCGGGCAGTGAGCGCAACGCAATTAATGTGAGTTAGCTCAC
TCATTAGGCACCCCAGGCTTTACACTTTATGCTTCCGGCTCGTATGTT
GTGTGGAATTGTGAGCGGATAACAATTTCACACAGGAAACAGCTAT
GACCATGATTACGCCAAGCGCGCAATTAACCCTCACTAAAGGGAAC
AAAAGCTGGAGCTGCAAGCTTGGCCATTGCATACGTTGTATCCATAT
CATAATATGTACATTTATATTGGCTCATGTCCAACATTACCGCCATG
TTGACATTGATTATTGACTAGTTATTAATAGTAATCAATTACGGGGT
CATTAGTTCATAGCCCATATATGG

| AGTTCCGCGTTACATAACTTACGGTAAATGGCCCGCCTGGCTGACCG | |
|---|---|
| CCCAACGACCCCCGCCCATTGACGTCAATAATGACGTATGTTCCCAT | |
| AGTAACGCCAATAGGGACTTTCCATTGACGTCAATGGGTGGAGTATT | |
| TACGGTAAACTGCCCACTTGGCAGTACATCAAGTGTATCATATGCCA | |
| AGTACGCCCCCTATTGACGTCAATGACGGTAAATGGCCCGCCTGGC | |
| ATTATGCCCAGTACATGACCTTATGGGACTTTCCTACTTGGCAGTAC | |
| ATCTACGTATTAGTCATCGCTATTACCATGGTGATGCGGTTTTGGCA | |
| GTACATCAATGGGCGTGGATAGCGGTTTGACTCACGGGGATTTCCA | |
| AGTCTCCACCCCATTGACGTCAATGGGAGTTTGTTTTGGCACCAAAA | |
| TCAACGGGACTTTCCAAAATGTCGTAACAACTCCGCCCCATTGACGC | |
| AAATGGGCGGTAGGCGTGTACGGTGGGAGGTCTATATAAGCAGAGC | |
| TCGTTTAGTGAACCG | |
| GGGTCTCTCTGGTTAGACCAGATCTGAGCCTGGGAGCTCTCTGGCTA | |
| ACTAGGGAACCCACTGCTTAAGCCTCAATAAAGCTTGCCTTGAGTGC | |
| TT | |
| CAAGTAGTGTGTGCCCGTCTGTTGTGTGACTCTGGTAACTAGAGATC | |
| CCTCAGACCCTTTTAGTCAGTGTGGAAAATCTCTAGC | |
| AGTGGCGCCCGAACAGGGACTTGAAAGCGAAAGGGAAACCAGAGG | |
| AGCTCTCTCGACGCAGGACTCGGCTTGCTGAAGCGCGCACGGCAAG | |
| AGGCGAGGGGCGGCGACTGGTGAGTACGCCAAAAATTTTGACTAGC | |
| GGAGGCTAGAAGGAGAGAGATGGGTGCGAGAGCGTCAGTATTAAG | |
| CGGGGGAGAATTAGATCGCGATGGGAAAAAATTCGGTTAAGGCCAG | |
| GGGGAAAGAAAAAATATAAATTAAAACATATAGTATGGGCAAGCA | |
| GGGAGCTAGAACGATTCGCAGTTAATCCTGGCCTGTTAGAAACATC | |
| AGAAGGCTGTAGACAAATACTGGGACAGCTACAACCATCCCTTCAG | |
| ACAGGATCAGAAGAACTTAGATCATTATATAATACAGTAGCAACCC | |
| TCTATTGTGTGCATCAAAGGATAGAGATAAAAGACACCAAGGAAGC | |
| TTTAGACAAGATAGAGGAAGAGCAAAACAAAAGTAAGACCACCGC | |
| ACAGCAAGCGGCCGCTGATCTTCAGACCTGGAGGAGGAGATATGAG | |
| GGACAATTGGAGAAGTGAATTATATAAATATAAAGTAGTAAAAATT | |
| GAACCATTAGG | |
| AGTAGCACCCACCAAGGCAAAGAGAAGAGTGGTGCAGAGAGAAAA | |
| AAGAGCAGTGGGAATAGGAGCTTTGTTCCTTGGGTTCTTGGGAGCA | |
| GCAGGAAGCACTATGGGCGCAGCGTCAATGACGCTGACGGTACAGG | |
| CCAGACAATTATTGTCTGGTATAGTGCAGCAGCAGAACAATTTGCTG | |
| AGGGCTATTGAGGCGCAACAGCATCTGTTGCAACTCACAGTCTGGG | |
| GCATCAAGCAGCTCCAGGCAAGAAT | |
| CCTGGCTGTGGAAAGATACCTAAAGGATCAACAGCTCCTGGGGATT | |
| TGGGGTTGCTCTGGAAAACTCATTTGCACCACTGCTGTGCCTTGGAA | |
| TGCTAGTTGGAGTAATAAATCTCTGGAACAGATTTGGAATCACACG | |
| ACCTGGATGGAGTGGGACAGAGAAATTAACAATTACACAAGCTTAA | |
| TACACTCCTTAATTGAAGAATCGCAAAACCAGCAAGAAAAGAATGA | |
| ACAAGAATTATTGGAATTAGATAAATGGGCAAGTTTGTGGAATTGG | |
| TTTAACATAACAAATTGGCTGTGGTATATAAAATTATTCATAATGAT | |
| AGTAGGAGGCTTGGTAGGTTTAAGAATAGTTTTTGCTGTACTTTCTA | |
| TAGTGAATAGAGTTAGGCAGGGATATTCACCATTATCGTTTCAGACC | |
| CACCTCCCAACCCCGAGGGGACCCGACAGGCCCGAAGGAATAGAAG | |
| AAGAAGGTGGAGAGAGAGACAGAGACAGATCCATTCGATTAGTGA | |
| ACGGATCTCGACGGTATCGATCTCGAC | |
| ACAAATGGCAGTATTCATCCACAATTTTAAAAGAAAAGGGGGGATT | |
| GGGGGGTACAGTGCAGGGGAAAGAATAGTAGACATAATAGCAACA | |
| GACATACAAACTAAAGAATTACAAAAACAAATTACAAAAATTCAAA | |
| ATTTTCGGGTTTATTACAGGGACAGCAGA | |
| CATGAGGACAGCTAAAACAATAAGTAATGTAAAATACAGCATAGCA | |
| AAACTTTAACCTCCAAATCAAGCCTCTACTTGAATCCTTTTCTGAGG | |
| GATGAATAAGGCATAGGCATCAGGGGCTGTTGCCAATGTGCATTAG | |
| CTGTTTGCAGCCTCACCTTCTTTCATGGAGTTTAAGATATAGTGTATT | |
| TTCCCAAGGTTTGAACTAGCTCTTCATTTCTTTATGTTTTAAATGCAC | |
| TGACCTCCCACATTCCCTTT | |
| TTAGTAAAATATTCAGAAATAATTTAAATACATCATTGCAATGAAAA | |
| TAAATGTTTTTTATTAGGCAGAATCCAGATGCTCAAGGCCCTTCATA | |
| ATATCCCCCAGTTTAGTAGTTGGACTTAGGGAACAAAGGAACCTTTA | |
| ATAGAAATTGGACAGCAAGAAAGCGAGC | |
| TTAGTGATACTTGTGGGCCAGGGCATTAGCCACACCAGCCACCACTT | |
| TCTGATAGGCAGCCTGCACTGGTGGGGTGAATTCTTTGCCAAAGTGA | |
| TGGGCCAGCACACAGACCAGCACGTTGCCCAGGAGCTGTGGGAGGA | |
| AGATAAGAG | |
| ATCTCTCCTGTCCTCCTTGTCAGGCTCCAGAGCTGGGGGTGCCCGGA | |
| CTGCTGAGCACTTAGTGTTTGTAGTGTCTCATCGCAAACTTACAAAC | |
| ACTAAGTGCTCAGCCAGCCAGGGCCCCACTGGGGGCACAGGAAGCA | |
| TGGCTTGAAAAGCG | |
| ACCTCGAGGACGCGTCGACGTC | |
| GGTGAGCATCCTTGTATCCAGCTGGCAGTCCATTCGTCATATCCCAT | |
| CTGCCCTGGCCTAGAGCTGGTAAGCATGTGCCGCACTGCGCGATCG | |
| AGTGTTGAATAACTCTACCACATGGAGTTATTCAACACTCGATCGCG | |
| CAGCGTGGCACTGCAGGAGGCCAGGCCAAGAGCTTCTGTGGGGAAG | |
| TGAGATC | |
| GTATGAACATGATTAGCAAAAGGGCCTAGCTTGGACTCAGAATAAT | |
| CCAGCCTTATCCCAACCATAAAATAAAAGCAGAATGGTAGCTGGAT | |
| TGTAGCTGCTATTAGCAATATGAAACCTCTTACATCAGTTACAATTT | |
| ATATGCAGAAATACCCTGTTACTTCTCCCCTTCCTATGACATGAACT | |
| TAACCATAGAAAAGAAGGGGAAAGAAAACATCAAGGGTCCCATAG | |
| ACTCACCCTGAAGTTCTCAGGATCCACGTGCAGCTTGTCACAGTGCA | |
| GCTCACTCAGCTGGGCAAAGGTGCCCTTGAGGTTGTCCAGGTGAGC | |
| CAGGCCATCACTAAAGGCACCGAGCACTTTCTTGCCATGAGCCTTCA | |
| CCTTAGGGTTGCCCATAACAGCATCAGGAGTGGACAGATCCCCAAA | |
| GGACTCAAAGAACCTCTGGGTCCAAGGGTAGACCACCAGCAGCCTA | |
| AGGGTGGGAAAATAGACCAATAGGCAGAGAGAGTCAGTGCCTATCA | |
| GAAACCCAAGAGTCTTCTCTGTCTCCACATGCCCAGTTTCTATTGGT | |
| CTCCTTAAACCTGTCTTGTAACCTTGATACCAACCTGCCCAGGGCCT | |
| CACCACCAACGGCATCCACGTTCACCTTGTCCCACAGGGCAGTAAC | |
| GGCAGACTTCTCCTCAGGAGTCAGGTGCACCAT | |
| GGTGTCTGTTTGAGGTTGCTAGTGAACACAGTTGTGTCAGAAGCAAA | |
| TGT | |
| AAGCAATAGATGGCTCTGCCCTGACTTTTATGCCCAGCCCTGGCTCC | |
| TGCCCTCCCTGCTCCTGGGAGTAGATTGGCCAACCCTAGGGTGTGGC | |
| TCCACAGGGTGAGGTCTAAGTGATGACAGCCGTACCTGTCCTTGGCT | |
| CTTCTGGCACTGGCTTAGGAGTTGGACTTCAAACCCTCAGCCCTCCC | |
| TCTAAGATATATCTCTTGGCCCCATACCATCAGTACAAATTGCTACT | |
| AAAAACATCCTCCTTTGCAA | |
| GTGTATTTACCATCAATAATTCTAGCCCCACAGGAGTTTGTTCTGAA | |
| AGTAAACTTCCACAACCGCAAGCTTATTGAGGCTAAGGCATCTGTG | |
| AAGGAAAGAAACATCTCCTCTAAACCACTATGCTGCTAGAGCCTCTT | |
| TTCTGTACTCAAGCCTCATTCAGACACTAGTGTCACCAGTCTCCTCA | |
| TATACCTATTGTATTTTCTTCTTCTTGCTGGTTTAGTCATGTTTTCTGG | |
| GAGCTTAGGGGCTTATTTTATTTTGTTTTGTTTTCTAATCAACAGAGA | |
| TGGGCAAACCCATTATTTTTTTCTTTAGACTTGGGATGGTGATAGCT | |
| GGGCAGCGTCAGAAACTGTGTGTGGATATAGATAAGAGCTCAGGAC | |
| TATGCTGAGCTGTGATGAGGGAGGGGCCTAGCTAAAGGCAGTGAGA | |
| GTCAGAATGCTCCTGCTATTGCCTTCTCAGTCCCCACGCTTGGTTTCT | |
| ACACAAGTAGATACATAGAAAAGGCTATAGGTTAGTGTTTGAGAGT | |
| CCTGCATGATTAGTTGCTCAGAAATGCCCGATAAATATGTTATGTGT | |
| GTTTATGTATATATATGTTTTATATGTGTGTGTGTGTGTGTTGTGTTT | |
| ACAAATATGTGATTATCATCAAAACGTGAGGGTACGTATATGTGTAT | |
| ATATATATATATATTCAGGAAATAATATATTCTAGAATATGTCACAT | |
| TCTGTCTCAGGCATCCATTTTCTTTATGATGCCGTTTGAGGTGGAGTT | |
| TTAGTCAGGTGGTCAGCTTCTCCTTTTTTTTGCCATCTGCCCTGTAAG | |
| CATCCTGCTGGGGACCCAGATAGGAGTCATCACTCTAGGCTGAGAA | |
| CATCTGGGCACACACCCTAAGCCTCAGCATGACTCATCATGACTCAG | |
| CATTGCTGTGCTTGAGCCAGAAGGTTTGCTTAGAAGGTTACACAGAA | |
| CCAGAAGGCGGGGGTGGGGCACTGACCCCGACAGGGGCCTGGCCA | |
| GAACTGCTCATGCTTGGACTATGGGAGGTCACTAATGGAGACACAC | |
| AGAAATGTAACAGGAACTAAGGAAAAACTGAAGCTTTGGGGGTATA | |
| GGGGAGCAGTCCCATGTAGTAGTAGAATGAAAAATGCTGCTATGCT | |
| GTGCCTCCCCCACCTTTCCCATGTCTGCCCTCTACTCATGGTCTATCT | |
| CTCCTGGCTCCTGGGAGTCATGGACTCCACCCAGCACCACCAACCTG | |
| ACCTAACCACCTATCTGAGCCTGCCAGCCTATAACCCATCTGGGCCC | |
| TGATAGCTGGTGGCCAGCCCTGACCCCACCCCACCCTCCCTGGAACC | |
| TCTGATAGACACATCTGGCACACCAGCTCGCAAAGTCACCGTGAGG | |
| GTCTTGTGTTTGCTGAGTCAAAATTCCTTGAAATCCAAGTCCTTAGA | |
| GACTCCCAGGCTTGGATTCAAAGCTCCTGACTTTCTGTCTAGTGTAT | |
| GTGCAGTGAGCCCCTTTTCCTCTAACTGAAAGAAGGAAAAAAAAAT | |
| GGAACCCAAAATATTCTACATAGTTTCCATGTCACAGCCAGGGCTGG | |
| GCAGTCTCCTGTTATTTCTTTTAAAATAAATATATCATTTAAATGCAT | |
| AAATAAGCAAACCCTGCTCGGGAATGGGAGGGAGAGTCTCTGGAGT | |
| CCACCCCTTCTCGGCCCTGGCTCTGCAGATAGTGCTATCAAAGCCCT | |
| GACAGAGCCCTGCCCATTGCTGGGCCTTGGAGTGAGTCAGCCTAGT | |
| AGAGAGGCAGGGCAAGCCATCTCATAGCTGCTGAGTGGGAGAGAG | |
| AAAAGGGCTCATTGTCTATAAACTCAGGTCATGGCTATTCTTAT | |
| TAAAAGAAAAGGGGGGAC | |
| TGGAAGGGCTAATTCACTCCCAACGAAGACAAGATCTGCTTTTTGCT | |
| TGTACT | |
| GGGTCTCTCTGGTTAGACCAGATCTGAGCCTGGGAGCTCTCTGGCTA | |
| ACTAGGGAACCCACTGCTTAAGCCTCAATAAAGCTTGCCTTGAGTGC | |
| TT | |
| CAAGTAGTGTGTTGGTTTTTTGTGTGTGCATTGTCTGAGTAGGTGTC | |
| ATTCTATTCTGGGGGGTGGGGTGGGGCAGCACAGCAAGGGGGAGGA | |
| TTGGGAAGACAATAGCAGGCATGCTGGGGATGCGGTGGGCTCTATG | |
| GAGTAGTAGTTCATGTCATCTTATTATTCAGTATTTATAACTTGCAA | |
| AGAAATGAATATCAGAGAGTGAGAGGAACTTGTTTATTGCAGCTTA | |
| TAATGGTTACAAATAAAGCAATAGCATCACAAATTTCACAAATAAA | |
| GCATTTTTTTCACTGCATTCTAGTTGTGGTTTGTCCAAACTCATCAAT | |
| GTATCTTATCATGTCTGGCTCTAGCTATCCCGCCCCTAACTCCGCCCA | |
| TCCCGCCCCTAACTCCGCCCAGTTCCGCCCATTCTCCGCCCCATGGC | |
| TCTCACTACTTCTGGAATAGCTCAGAGGCCGAGGCGGCCTCGGCCTC | |
| TGCATAAATAAAAAAAATTAGTCGAGGCTTTTTTGGAGGCCTAGGG | |
| ACGTACCCAATTCGCCCTATAGTGAGTCGTATTACGCGCGCTCACTG | |
| GCCGTCGTTTTACAACGTCGTGACTGGGAAAACCCTGGCGTTACCCA | |
| ACTTAATCGCCTTGCAGCACATCCCCCTTTCGCCAGCTGGCGTAATA | |
| GCGAAGAGGCCCGCACCGATCGCCCTTCCCAACAGTTGCGCAGCCT | |
| GAATGGCGAATGGG | |
| XXXXXXXX | CMV |
| XXXXXXXX | R |
| XXXXXXXX | U5 |
| XXXXXXXX | RRE |
| XXXXXXXX | cPPT |
| 3′UTR | 3′UTR |
| AS3 globin | AS3 globin |
| XXXXXXXX | ZNF410 shmiR in miR 144 (reverse complement) |
| XXXXXXXX | BCL11A shmiR in miR223 (reverse complement) |
| XXXXXXXX | Globin-promoter |
| XXXXXXXX | LCR |
| XXXXXXXX | delta U3 |

TABLE 10
Mini-G del IVS2 shmiR BCL11A-ZNF410 (SEQ ID NO: 18)
(see, e.g., FIG. 4, panel B).

| ACGCGCCCTGTAGCGGCGCATTAAGCGCGGCGGGTGTGGTGGTTAC | | CMV |
|---|---|---|
| GCGCAGCGTGACCGCTACACTTGCCAGCGCCCTAGCGCCCGCTCCTT | | R |
| TCGCTTTCTTCCCTTCCTTTCTCGCCACGTTCGCCGGCTTTCCCCGTC | | U5 |
| AAGCTCTAAATCGGGGGCTCCCTTTAGGGTTCCGATTTAGTGCTTTA | | RRE |
| CGGCACCTCGACCCCAAAAAACTTGATTAGGGTGATGGTTCACGTA | | CPPT |
| GTGGGCCATCGCCCTGATAGACGGTTTTTCGCCCTTTGACGTTGGAG | | 3′UTR |
| TCCACGTTCTTTAATAGTGGACTCTTGTTCCAAACTGGAACAACACT | | ZNF410 |
| CAACCCTATCTCGGTCTATTCTTTTGATTTATAAGGGATTTTGCCGAT | | shmiR in |
| TTCGGCCTATTGGTTAAAAAATGAGCTGATTTAACAAAAATTTAACG | | miR144 |
| CGAATTTTAACAAAATATTAACGCTTACAATTTAGGTGGCACTTTTC | | Rev |
| GGGGAAATGTGCGCGGAACCCCTATTTGTTTATTTTTCTAAATACAT | | comp |
| TCAAATATGTATCCGCTCATGAGACAATAACCCTGATAAATGCTTCA | | BCL11A |
| ATAATAGCACCTAGATCAAGAGACAGGATGAGGATCGTTTCGCATG | | shmiR in |
| ATTGAACAAGATGGATTGCACGCAGGTTCTCCGGCCGCTTGGGTGG | | miR223 |
| AGAGGCTATTCGGCTATGACTGGGCACAACAGACAATCGGCTGCTC | | Rev |
| TGATGCCGCCGTGTTCCGGCTGTCAGCGCAGGGGCGCCCGGTTCTTT | | comp |
| TTGTCAAGACCGACCTGTCCGGTGCCCTGAATGAACTGCAAGACGA | | Globin- |
| GGCAGCGCGGCTATCGTGGCTGGCCACGACGGGCGTTCCTTGCGCA | | promoter |
| GCTGTGCTCGACGTTGTCACTGAAGCGGGAAGGGACTGGCTGCTATT | | LCR |
| GGGCGAAGTGCCGGGGCAGGATCTCCTGTCATCTCACCTTGCTCCTG | | Delta U3 |
| CCGAGAAAGTATCCATCATGGCTGATGCAATGCGGCGGCTGCATAC | | R |
| GCTTGATCCGGCTACCTGCCCATTCGACCACCAAGCGAAACATCGCA | | |
| TCGAGCGAGCACGTACTCGGATGGAAGCCGGTCTTGTCGATCAGGA | | |
| TGATCTGGACGAAGAGCATCAGGGGCTCGCGCCAGCCGAACTGTTC | | |
| GCCAGGCTCAAGGCGAGCATGCCCGACGGCGAGGATCTCGTCGTGA | | |
| CCCATGGCGATGCCTGCTTGCCGAATATCATGGTGGAAAATGGCCG | | |
| CTTTTCTGGATTCATCGACTGTGGCCGGCTGGGTGTGGCGGACCGCT | | |
| ATCAGGACATAGCGTTGGCTACCCGTGATATTGCTGAAGAGCTTGGC | | |
| GGCGAATGGGCTGACCGCTTCCTCGTGCTTTACGGTATCGCCGCTCC | | |
| CGATTCGCAGCGCATCGCCTTCTATCGCCTTCTTGACGAGTTCTTCTG | | |
| AATTATTAACGCTTACAATTTCCTGATGCGGTATTTTCTCCTTACGCA | | |
| TCTGTGCGGTATTTCACACCGCATCAGGTGGCACTTTTCGGGGAAAT | | |
| GTGCGCGGAACCCCTATTTGTTTATTTTTCTAAATACATTCAAATATG | | |
| TATCCGCTCATGACCAAAATCCCTTAACGTGAGTTTTCGTTCCACTG | | |
| AGCGTCAGACCCCGTAGAAAAGATCAAAGGATCTTCTTGAGATCCT | | |
| TTTTTTCTGCGCGTAATCTGCTGCTTGCAAACAAAAAAACCACCGCT | | |
| ACCAGCGGTGGTTTGTTTGCCGGATCAAGAGCTACCAACTCTTTTTC | | |
| CGAAGGTAACTGGCTTCAGCAGAGCGCAGATACCAAATACTGTTCT | | |
| TCTAGTGTAGCCGTAGTTAGGCCACCACTTCAAGAACTCTGTAGCAC | | |
| CGCCTACATACCTCGCTCTGCTAATCCTGTTACCAGTGGCTGCTGCC | | |
| AGTGGCGATAAGTCGTGTCTTACCGGGTTGGACTCAAGACGATAGTT | | |
| ACCGGATAAGGCGCAGCGGTCGGGCTGAACGGGGGGGTCGTGCACA | | |
| CAGCCCAGCTTGGAGCGAACGACCTACACCGAACTGAGATACCTAC | | |
| AGCGTGAGCTATGAGAAAGCGCCACGCTTCCCGAAGGGAGAAAGGC | | |
| GGACAGGTATCCGGTAAGCGGCAGGGTCGGAACAGGAGAGCGCAC | | |
| GAGGGAGCTTCCAGGGGGAAACGCCTGGTATCTTTATAGTCCTGTCG | | |
| GGTTTCGCCACCTCTGACTTGAGCGTCGATTTTTGTGATGCTCGTCA | | |
| GGGGGGCGGAGCCTATGGAAAAACGCCAGCAACGCGGCCTTTTTAC | | |
| GGTTCCTGGCCTTTTGCTGGCCTTTTGCTCACATGTTCTTTCCTGCGT | | |
| TATCCCCTGATTCTGTGGATAACCGTATTACCGCCTTTGAGTGAGCT | | |
| GATACCGCTCGCCGCAGCCGAACGACCGAGCGCAGCGAGTCAGTGA | | |
| GCGAGGAAGCGGAAGAGCGCCCAATACGCAAACCGCCTCTCCCCGC | | |
| GCGTTGGCCGATTCATTAATGCAGCTGGCACGACAGGTTTCCCGACT | | |
| GGAAAGCGGGCAGTGAGCGCAACGCAATTAATGTGAGTTAGCTCAC | | |
| TCATTAGGCACCCCAGGCTTTACACTTTATGCTTCCGGCTCGTATGTT | | |
| GTGTGGAATTGTGAGCGGATAACAATTTCACACAGGAAACAGCTAT | | |
| GACCATGATTACGCCAAGCGCGCAATTAACCCTCACTAAAGGGAAC | | |
| AAAAGCTGGAGCTGCAAGCTTGGCCATTGCATACGTTGTATCCATAT | | |
| CATAATATGTACATTTATATTGGCTCATGTCCAACATTACCGCCATG | | |
| TTGACATTGATTATTGACTAGTTATTAATAGTAATCAATTACGGGGT | | |
| CATTAGTTCATAGCCCATATATGG | | |
| AGTTCCGCGTTACATAACTTACGGTAAATGGCCCGCCTGGCTGACCG | | |
| CCCAACGACCCCCGCCCATTGACGTCAATAATGACGTATGTTCCCAT | | |
| AGTAACGCCAATAGGGACTTTCCATTGACGTCAATGGGTGGAGTATT | | |
| TACGGTAAACTGCCCACTTGGCAGTACATCAAGTGTATCATATGCCA | | |
| AGTACGCCCCCTATTGACGTCAATGACGGTAAATGGCCCGCCTGGC | | |
| ATTATGCCCAGTACATGACCTTATGGGACTTTCCTACTTGGCAGTAC | | |
| ATCTACGTATTAGTCATCGCTATTACCATGGTGATGCGGTTTTGGCA | | |
| GTACATCAATGGGCGTGGATAGCGGTTTGACTCACGGGGATTTCCA | | |
| AGTCTCCACCCCATTGACGTCAATGGGAGTTTGTTTTGGCACCAAAA | | |
| TCAACGGGACTTTCCAAAATGTCGTAACAACTCCGCCCCATTGACGC | | |
| AAATGGGCGGTAGGCGTGTACGGTGGGAGGTCTATATAAGCAGAGC | | |
| TCGTTTAGTGAACCG | | |
| GGGTCTCTCTGGTTAGACCAGATCTGAGCCTGGGAGCTCTCTGGCTA | | |
| ACTAGGGAACCCACTGCTTAAGCCTCAATAAAGCTTGCCTTGAGTGC | | |
| TT | | |
| CAAGTAGTGTGTGCCCGTCTGTTGTGTGACTCTGGTAACTAGAGATC | | |
| CCTCAGACCCTTTTAGTCAGTGTGGAAAATCTCTAGC | | |
| AGTGGCGCCCGAACAGGGACTTGAAAGCGAAAGGGAAACCAGAGG | | |
| AGCTCTCTCGACGCAGGACTCGGCTTGCTGAAGCGCGCACGGCAAG | | |
| AGGCGAGGGGCGGCGACTGGTGAGTACGCCAAAAATTTTGACTAGC | | |
| GGAGGCTAGAAGGAGAGAGATGGGTGCGAGAGCGTCAGTATTAAG | | |
| CGGGGGAGAATTAGATCGCGATGGGAAAAAATTCGGTTAAGGCCAG | | |
| GGGGAAAGAAAAAATATAAATTAAAACATATAGTATGGGCAAGCA | | |
| GGGAGCTAGAACGATTCGCAGTTAATCCTGGCCTGTTAGAAACATC | | |
| AGAAGGCTGTAGACAAATACTGGGACAGCTACAACCATCCCTTCAG | | |
| ACAGGATCAGAAGAACTTAGATCATTATATAATACAGTAGCAACCC | | |
| TCTATTGTGTGCATCAAAGGATAGAGATAAAAGACACCAAGGAAGC | | |
| TTTAGACAAGATAGAGGAAGAGCAAAACAAAAGTAAGACCACCGC | | |
| ACAGCAAGCGGCCGCTGATCTTCAGACCTGGAGGAGGAGATATGAG | | |
| GGACAATTGGAGAAGTGAATTATATAAATATAAAGTAGTAAAAATT | | |
| GAACCATTAGG | | |
| AGTAGCACCCACCAAGGCAAAGAGAAGAGTGGTGCAGAGAGAAAA | | |
| AAGAGCAGTGGGAATAGGAGCTTTGTTCCTTGGGTTCTTGGGAGCA | | |
| GCAGGAAGCACTATGGGCGCAGCGTCAATGACGCTGACGGTACAGG | | |
| CCAGACAATTATTGTCTGGTATAGTGCAGCAGCAGAACAATTTGCTG | | |
| AGGGCTATTGAGGCGCAACAGCATCTGTTGCAACTCACAGTCTGGG | | |
| GCATCAAGCAGCTCCAGGCAAGAAT | | |
| CCTGGCTGTGGAAAGATACCTAAAGGATCAACAGCTCCTGGGGATT | | |
| TGGGGTTGCTCTGGAAAACTCATTTGCACCACTGCTGTGCCTTGGAA | | |
| TGCTAGTTGGAGTAATAAATCTCTGGAACAGATTTGGAATCACACG | | |
| ACCTGGATGGAGTGGGACAGAGAAATTAACAATTACACAAGCTTAA | | |
| TACACTCCTTAATTGAAGAATCGCAAAACCAGCAAGAAAAGAATGA | | |
| ACAAGAATTATTGGAATTAGATAAATGGGCAAGTTTGTGGAATTGG | | |
| TTTAACATAACAAATTGGCTGTGGTATATAAAATTATTCATAATGAT | | |
| AGTAGGAGGCTTGGTAGGTTTAAGAATAGTTTTTGCTGTACTTTCTA | | |
| TAGTGAATAGAGTTAGGCAGGGATATTCACCATTATCGTTTCAGACC | | |
| CACCTCCCAACCCCGAGGGGACCCGACAGGCCCGAAGGAATAGAAG | | |
| AAGAAGGTGGAGAGAGAGACAGAGACAGATCCATTCGATTAGTGA | | |
| ACGGATCTCGACGGTATCGATCTCGAC | | |
| ACAAATGGCAGTATTCATCCACAATTTTAAAAGAAAAGGGGGGATT | | |
| GGGGGGTACAGTGCAGGGGAAAGAATAGTAGACATAATAGCAACA | | |
| GACATACAAACTAAAGAATTACAAAAACAAATTACAAAAATTCAAA | | |
| ATTTTCGGGTTTATTACAGGGACAGCAGA | | |
| CATGAGGACAGCTAAAACAATAAGTAATGTAAAATACAGCATAGCA | | |
| AAACTTTAACCTCCAAATCAAGCCTCTACTTGAATCCTTTTCTGAGG | | |
| GATGAATAAGGCATAGGCATCAGGGGCTGTTGCCAATGTGCATTAG | | |
| CTGTTTGCAGCCTCACCTTCTTTCATGGAGTTTAAGATATAGTGTATT | | |
| TTCCCAAGGTTTGAACTAGCTCTTCATTTCTTTATGTTTTAAATGCAC | | |
| TGACCTCCCACATTCCCTTT | | |
| TTAGTAAAATATTCAGAAATAATTTAAATACATCATTGCAATGAAAA | | |
| TAAATGTTTTTTATTAGGCAGAATCCAGATGCTCAAGGCCCTTCATA | | |
| ATATCCCCCAGTTTAGTAGTTGGACTTAGGGAACAAAGGAACCTTTA | | |
| ATAGAAATTGGACAGCAAGAAAGCGAGC | | |
| TTAGTGATACTTGTGGGCCAGGGCATTAGCCACACCAGCCACCACTT | | |
| TCTGATAGGCAGCCTGCACTGGTGGGGTGAATTCTTTGCCAAAGTGA | | |
| TGGGCCAGCACACAGACCAGCACGTTGCCCAGGAGCTGTGGGAGGA | | |
| AGATAAGAGGTATGAACATGATTAGCAAAAGGGCCTAGCTTGGACT | | |
| CAGAATAATCCAGCCTTATCCCAACCATAAAATAAAAGCAGAATGG | | |
| TAGCTGGATTGTAGCTGCTATTAGCAATATGAAACCTCTTACATCAG | | |
| TTACAATTTATATGCAGAAATA | | |
| ATCTCTCCTGTCCTCCTTGTCAGGCTCCAGAGCTGGGGGTGCCCGGA | | |
| CTGCTGAGCACTTAGTGTTTGTAGTGTCTCATCGCAAACTTACAAAC | | |
| ACTAAGTGCTCAGCCAGCCAGGGCCCCACTGGGGGCACAGGAAGCA | | |
| TGGCTTGAAAAGCG | | |
| ACCTCGAGGACGCGTCGACGTC | | |
| GGTGAGCATCCTTGTATCCAGCTGGCAGTCCATTCGTCATATCCCAT | | |
| CTGCCCTGGCCTAGAGCTGGTAAGCATGTGCCGCACTGCGCGATCG | | |
| AGTGTTGAATAACTCTACCACATGGAGTTATTCAACACTCGATCGCG | | |
| CAGCGTGGCACTGCAGGAGGCCAGGCCAAGAGCTTCTGTGGGGAAG | | |
| TGAGATC | | |
| CCCTGTTACTTCTCCCCTTCCTATGACATGAACTTAACCATAGAAAA | | |
| GAAGGGGAAAGAAAACATCAAGGGTCCCATAGACTCACCCTGAAGT | | |
| TCTCAGGATCCACGTGCAGCTTGTCACAGTGCAGCTCACTCAGCTGG | | |
| GCAAAGGTGCCCTTGAGGTTGTCCAGGTGAGCCAGGCCATCACTAA | | |
| AGGCACCGAGCACTTTCTTGCCATGAGCCTTCACCTTAGGGTTGCCC | | |
| ATAACAGCATCAGGAGTGGACAGATCCCCAAAGGACTCAAAGAACC | | |
| TCTGGGTCCAAGGGTAGACCACCAGCAGCCTAAGGGTGGGAAAATA | | |
| GACCAATAGGCAGAGAGAGTCAGTGCCTATCAGAAACCCAAGAGTC | | |
| TTCTCTGTCTCCACATGCCCAGTTTCTATTGGTCTCCTTAAACCTGTC | | |
| TTGTAACCTTGATACCAACCTGCCCAGGGCCTCACCACCAACGGCAT | | |
| CCACGTTCACCTTGTCCCACAGGGCAGTAACGGCAGACTTCTCCTCA | | |
| GGAGTCAGGTGCACCAT | | |
| GGTGTCTGTTTGAGGTTGCTAGTGAACACAGTTGTGTCAGAAGCAAA | | |
| TGT | | |
| AAGCAATAGATGGCTCTGCCCTGACTTTTATGCCCAGCCCTGGCTCC | | |
| TGCCCTCCCTGCTCCTGGGAGTAGATTGGCCAACCCTAGGGTGTGGC | | |
| TCCACAGGGTGAGGTCTAAGTGATGACAGCCGTACCTGTCCTTGGCT | | |
| CTTCTGGCACTGGCTTAGGAGTTGGACTTCAAACCCTCAGCCCTCCC | | |
| TCTAAGATATATCTCTTGGCCCCATACCATCAGTACAAATTGCTACT | | |
| AAAAACATCCTCCTTTGCAA | | |
| GTGTATTTACCATCAATAATTCTAGCCCCACAGGAGTTTGTTCTGAA | | |
| AGTAAACTTCCACAACCGCAAGCTTATTGAGGCTAAGGCATCTGTG | | |
| AAGGAAAGAAACATCTCCTCTAAACCACTATGCTGCTAGAGCCTCTT | | |
| TTCTGTACTCAAGCCTCATTCAGACACTAGTGTCACCAGTCTCCTCA | | |
| TATACCTATTGTATTTTCTTCTTCTTGCTGGTTTAGTCATGTTTTCTGG | | |
| GAGCTTAGGGGCTTATTTTATTTTGTTTTGTTTTCTAATCAACAGAGA | | |
| TGGGCAAACCCATTATTTTTTTCTTTAGACTTGGGATGGTGATAGCT | | |
| GGGCAGCGTCAGAAACTGTGTGTGGATATAGATAAGAGCTCAGGAC | | |
| TATGCTGAGCTGTGATGAGGGAGGGGCCTAGCTAAAGGCAGTGAGA | | |
| GTCAGAATGCTCCTGCTATTGCCTTCTCAGTCCCCACGCTTGGTTTCT | | |
| ACACAAGTAGATACATAGAAAAGGCTATAGGTTAGTGTTTGAGAGT | | |
| CCTGCATGATTAGTTGCTCAGAAATGCCCGATAAATATGTTATGTGT | | |
| GTTTATGTATATATATGTTTTATATGTGTGTGTGTGTGTGTTGTGTTT | | |
| ACAAATATGTGATTATCATCAAAACGTGAGGGTACGTATATGTGTAT | | |
| ATATATATATATATTCAGGAAATAATATATTCTAGAATATGTCACAT | | |
| TCTGTCTCAGGCATCCATTTTCTTTATGATGCCGTTTGAGGTGGAGTT | | |
| TTAGTCAGGTGGTCAGCTTCTCCTTTTTTTTGCCATCTGCCCTGTAAG | | |
| CATCCTGCTGGGGACCCAGATAGGAGTCATCACTCTAGGCTGAGAA | | |
| CATCTGGGCACACACCCTAAGCCTCAGCATGACTCATCATGACTCAG | | |
| CATTGCTGTGCTTGAGCCAGAAGGTTTGCTTAGAAGGTTACACAGAA | | |
| CCAGAAGGCGGGGGTGGGGCACTGACCCCGACAGGGGCCTGGCCA | | |
| GAACTGCTCATGCTTGGACTATGGGAGGTCACTAATGGAGACACAC | | |
| AGAAATGTAACAGGAACTAAGGAAAAACTGAAGCTTTGGGGGTATA | | |
| GGGGAGCAGTCCCATGTAGTAGTAGAATGAAAAATGCTGCTATGCT | | |
| GTGCCTCCCCCACCTTTCCCATGTCTGCCCTCTACTCATGGTCTATCT | | |
| CTCCTGGCTCCTGGGAGTCATGGACTCCACCCAGCACCACCAACCTG | | |
| ACCTAACCACCTATCTGAGCCTGCCAGCCTATAACCCATCTGGGCCC | | |
| TGATAGCTGGTGGCCAGCCCTGACCCCACCCCACCCTCCCTGGAACC | | |
| TCTGATAGACACATCTGGCACACCAGCTCGCAAAGTCACCGTGAGG | | |
| GTCTTGTGTTTGCTGAGTCAAAATTCCTTGAAATCCAAGTCCTTAGA | | |
| GACTCCCAGGCTTGGATTCAAAGCTCCTGACTTTCTGTCTAGTGTAT | | |
| GTGCAGTGAGCCCCTTTTCCTCTAACTGAAAGAAGGAAAAAAAAAT | | |
| GGAACCCAAAATATTCTACATAGTTTCCATGTCACAGCCAGGGCTGG | | |
| GCAGTCTCCTGTTATTTCTTTTAAAATAAATATATCATTTAAATGCAT | | |
| AAATAAGCAAACCCTGCTCGGGAATGGGAGGGAGAGTCTCTGGAGT | | |
| CCACCCCTTCTCGGCCCTGGCTCTGCAGATAGTGCTATCAAAGCCCT | | |
| GACAGAGCCCTGCCCATTGCTGGGCCTTGGAGTGAGTCAGCCTAGT | | |
| AGAGAGGCAGGGCAAGCCATCTCATAGCTGCTGAGTGGGAGAGAG | | |
| AAAAGGGCTCATTGTCTATAAACTCAGGTCATGGCTATTCTTATTAA | | |
| AAGAAAAGGGGGGAC | | |
| TGGAAGGGCTAATTCACTCCCAACGAAGACAAGATCTGCTTTTTGCT | | |
| TGTACT | | |
| GGGTCTCTCTGGTTAGACCAGATCTGAGCCTGGGAGCTCTCTGGCTA | | |
| ACTAGGGAACCCACTGCTTAAGCCTCAATAAAGCTTGCCTTGAGTGC | | |
| TT | | |
| CAAGTAGTGTGTTGGTTTTTTGTGTGTGCATTGTCTGAGTAGGTGTC | | |
| ATTCTATTCTGGGGGGTGGGGTGGGGCAGCACAGCAAGGGGGAGGA | | |
| TTGGGAAGACAATAGCAGGCATGCTGGGGATGCGGTGGGCTCTATG | | |
| GAGTAGTAGTTCATGTCATCTTATTATTCAGTATTTATAACTTGCAA | | |
| AGAAATGAATATCAGAGAGTGAGAGGAACTTGTTTATTGCAGCTTA | | |
| TAATGGTTACAAATAAAGCAATAGCATCACAAATTTCACAAATAAA | | |
| GCATTTTTTTCACTGCATTCTAGTTGTGGTTTGTCCAAACTCATCAAT | | |
| GTATCTTATCATGTCTGGCTCTAGCTATCCCGCCCCTAACTCCGCCCA | | |
| TCCCGCCCCTAACTCCGCCCAGTTCCGCCCATTCTCCGCCCCATGGC | | |
| TCTCACTACTTCTGGAATAGCTCAGAGGCCGAGGCGGCCTCGGCCTC | | |
| TGCATAAATAAAAAAAATTAGTCGAGGCTTTTTTGGAGGCCTAGGG | | |
| ACGTACCCAATTCGCCCTATAGTGAGTCGTATTACGCGCGCTCACTG | | |
| GCCGTCGTTTTACAACGTCGTGACTGGGAAAACCCTGGCGTTACCCA | | |
| ACTTAATCGCCTTGCAGCACATCCCCCTTTCGCCAGCTGGCGTAATA | | |
| GCGAAGAGGCCCGCACCGATCGCCCTTCCCAACAGTTGCGCAGCCT | | |
| GAATGGCGAATGGG | | |
| XXXXXXXX | CMV | |
| XXXXXXXX | R | |
| XXXXXXXX | U5 | |
| XXXXXXXX | RRE | |
| XXXXXXXX | cPPT | |
| 3′UTR | 3′UTR | |
| AS3 globin | AS3 globin | |
| XXXXXXXX | ZNF410 shmiR in miR144 (reverse complement) | |
| XXXXXXXX | BCL11A shmiR in miR223 (reverse complement) | |
| XXXXXXXX | Globin-promoter | |
| XXXXXXXX | LCR | |
| XXXXXXXX | U3 | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A recombinant lentiviral vector (LV) comprising:
an expression cassette that encodes that an anti-sickling β-globin gene wherein said expression cassette is in reverse orientation in said vector;
a β-globin locus control region (LCR) comprising a reduced length hypersensitive site 2 (HS2) sequence, a reduced length hypersensitive site 3 (HS3) sequence, and a reduced length hypersensitive site 4 (HS4) sequence, where said anti-sickling β-globin gene is operably linked to said human β-globin locus control region; and
an shRNA in a microRNA scaffold (shmiR), that inhibits expression of the BCL11A gene (BCL11A shmiR) and/or an shRNA in a microRNA scaffold (shmiR), that inhibits expression of a ZNF410 gene (ZNF410 shmiR).

2. The lentiviral vector of claim 1, wherein said vector comprises a globin promoter.

3. The lentiviral vector according to claim 1, wherein said vector comprises a BCL11A shmiR.

4. The lentiviral vector according to claim 1, wherein said reduced length hypersensitive site 2 (HS2) sequence consists of the nucleotide sequence of EC2 set forth in SEQ ID NO:4.

5. The lentiviral vector according to claim 1, wherein said reduced length hypersensitive site 3 (HS3) sequence consists of the nucleotide sequence of EC3 set forth in SEQ ID NO:5.

6. The lentiviral vector according to claim 1, wherein said reduced length hypersensitive site 4 (HS4) sequence consists of the nucleotide sequence of EC4 set forth in SEQ ID NO:6.

7. The lentiviral vector according to claim 1, wherein said vector comprises a reduced length hypersensitive site 1 (HS1) sequence.

8. The lentiviral vector of claim 1, wherein said reduced length hypersensitive site 1 (HS1) sequence consists of the nucleotide sequence of EC1 set forth in SEQ ID NO:7.

9. The lentiviral vector according to claim 1, wherein said anti-sickling human beta globin gene encoding an anti-sickling-beta globin polypeptide comprise one or more mutations selected from the group consisting of Gly16Asp, Glu22Ala, and Thr87Gln.

10. The lentiviral vector of claim 9, wherein said beta globin gene comprises the mutation Gly16Asp.

11. The lentiviral vector according to claim 9, wherein said beta globin gene comprises the mutation Glu22Ala.

12. The lentiviral vector according to claim 9, wherein said beta globin gene comprises the mutation Thr87Gln.

13. The lentiviral vector of claim 9, wherein said anti-sickling human β-globin gene comprises about 2.3 kb of recombinant human β-globin gene including exons and introns under the control of said human β-globin locus control region.

14. The lentiviral vector according to claim 1, wherein said β-globin gene comprises βAS3 comprising an intervening sequence I (IVS1) and an intervening sequence 2 (IVS).

15. The lentiviral vector of claim 14, wherein said β-globin gene comprises β-globin intron 2 with a 375 bp RsaI deletion from IVS2.

16. The lentiviral vector claim 14, wherein said β-globin gene comprises an SspI (S) to RsaI (R) deletion (˜220 bp).

17. The lentiviral vector claim 14, wherein said β-globin gene comprises a 591 bp deletion in IVS2.

18. The lentiviral vector according to claim 1, wherein said BCL11A shmiR comprises a BCL11A shRNA embedded in a microRNA sequence.

19. The lentiviral vector of claim 18, wherein said BCL11A shRNA is embedded in microRNA 223 (MIR223).

20. The lentiviral vector of claim 19, wherein said BCL11A shmiR comprises the nucleotide sequence of SEQ ID NO:8.

21. The lentiviral vector according to claim 1, wherein said BCL11A shmiR is located upstream or downstream of said anti-sickling β-globin gene.

22. The lentiviral vector according to claim 1, wherein said BCL11A shmiR is disposed in a noncoding sequence of said anti-sickling β-globin gene.

23. The lentiviral vector according to claim 14, wherein said BCL11A shmiR is disposed in IVS1 and/or IVS2.

24. The lentiviral vector of claim 23, wherein said BCL11A shmiR is disposed in IVS1.

25. The lentiviral vector of claim 24, wherein said BCL11A shmiR is disposed in the upstream half or quarter of IVS1.

26. The lentiviral vector of claim 24, wherein said BCL11A shmiR is disposed in the downstream half or quarter of IVS1.

27. The lentiviral vector of claim 23, wherein said BCL11A shmiR is disposed in IVS2.

28. The lentiviral vector of claim 27, wherein said BCL11A shmiR is disposed in the upstream half or quarter of IVS2.

29. The lentiviral vector of claim 27, wherein said BCL11A shmiR is disposed in the downstream half or quarter of IVS2.

30. The lentiviral vector of claim 27, wherein said BCL11A shmiR is disposed at or downstream from a deletion in IVS2.

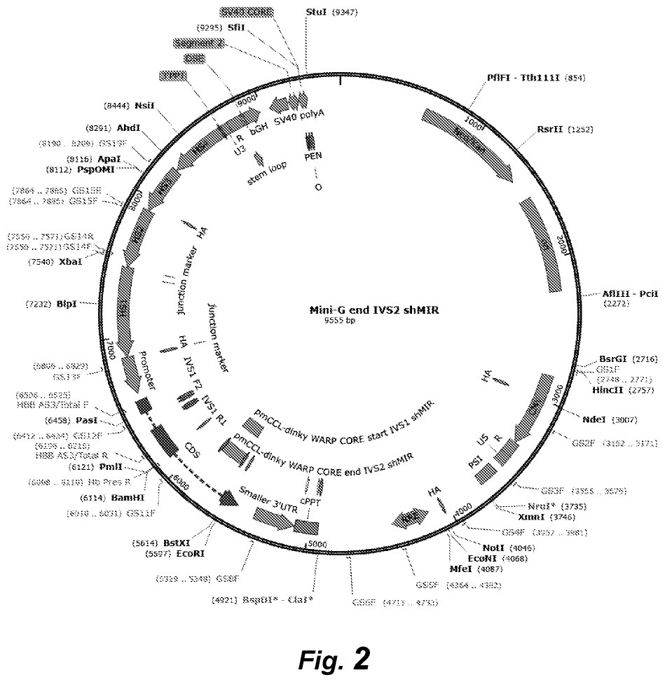

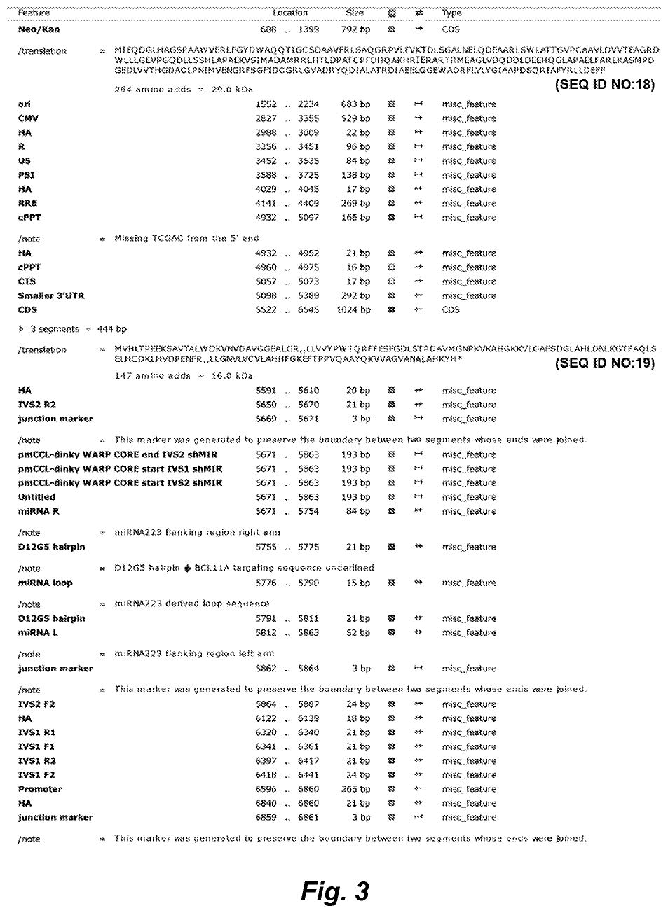

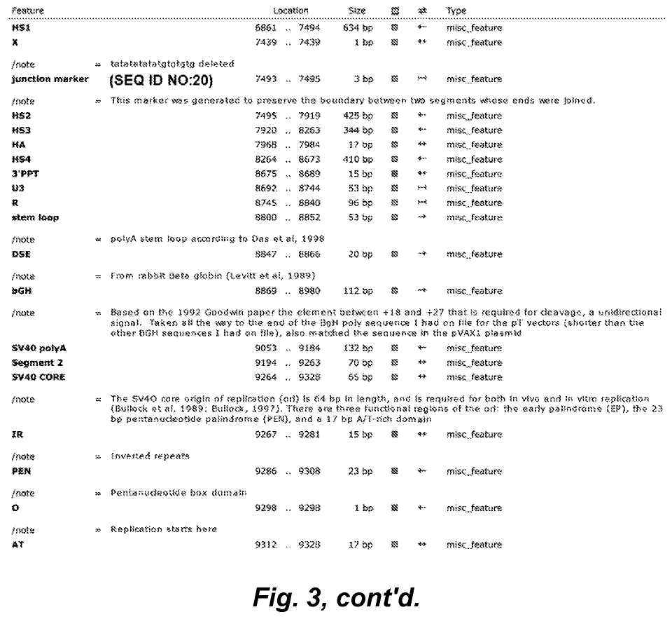

31. The lentiviral vector according to claim 1, wherein said vector comprises features shown and located as in the vector map in FIG. 2 and/or the feature list in FIG. 3.

32. The lentiviral vector of claim 1, wherein said vector comprises the nucleic acid sequence of SEQ ID NO:12.

33. The lentiviral vector of claim 1, wherein said vector comprises the nucleic acid sequence of SEQ ID NO:13.

34. The lentiviral vector of claim 1, wherein said vector comprises the nucleic acid sequence of SEQ ID NO:14.

35. The lentiviral vector of claim 1, wherein said vector comprises the nucleic acid sequence of SEQ ID NO:15.

36. The lentiviral vector according to claim 1, wherein said vector comprises a ZNF410 shRNA).

37. The lentiviral vector of claim 36, wherein said ZNF410 shmiR comprises a ZNF410 shRNA embedded in microRNA 144 (MIR144).

38. The lentiviral vector of claim 37, wherein said ZNF410 shmiR comprises the nucleotide sequence of SEQ ID NO:10.

39. The lentiviral vector according to claim 36, wherein said ZNF410 shmiRis located upstream or downstream of said anti-sickling β-globin gene.

40. The lentiviral vector according to claim 36, wherein said ZNF410 shmiR is disposed in a noncoding sequence of said anti-sickling β-globin gene.

41. The lentiviral vector according to claim 14, wherein said ZNF410 shmiR is disposed in IVS1 and/or IVS2.

42. The lentiviral vector of claim 41, wherein said ZNF410 shmiR is disposed in IVS1.

43. The lentiviral vector of claim 42, wherein said ZNF410 shmiRis disposed in the upstream half or quarter of IVS1.

44. The lentiviral vector of claim 42, wherein said ZNF410 shmiR is disposed in the downstream half or quarter of IVS1.

45. The lentiviral vector of claim 41, wherein said ZNF410 shmiR is disposed in IVS2.

46. The lentiviral vector of claim 45, wherein said ZNF410 shmiR is disposed in the upstream half or quarter of IVS2.

47. The lentiviral vector of claim 45, wherein said ZNF410 shmiR is disposed in the downstream half or quarter of IVS2.

48. The lentiviral vector of claim 45, wherein said ZNF410 shmiR is disposed at or downstream from a deletion in IVS2.

49. The lentiviral vector according to claim 36, wherein said vector comprises features shown and located as shown in FIG. 4, panel A or panel B.

50. The lentiviral vector of claim 1, wherein said vector comprises the nucleic acid sequence of SEQ ID NO:17.

51. The lentiviral vector of claim 1, wherein said vector comprises the nucleic acid sequence of SEQ ID NO: 18

52. The lentiviral vector according to claim 1, wherein said vector when transduced into a human cell is capable of maintaining % βAS3/VCN expression while increasing fetal globin expression by 10-15 fold.

53. A host cell transduced with a vector according to claim 1.

54. The host cell of claim 53, wherein the cell is a stem cell.

55. The host cell of claim 54, wherein said cell is a stem cell derived from bone marrow, and/or from umbilical cord blood, and/or from peripheral blood.

56. The host cell of claim 53, wherein the cell is a 293T cell.

57. The host cell of claim 53, wherein, wherein the cell is a human hematopoietic progenitor cell.

58. The host cell of claim 57, wherein the human hematopoietic progenitor cell is a CD34+ cell.

59. A method of treating a sickle cell disease (SCD), in a subject, said method comprising:
transducing a stem cell and/or progenitor cell from said subject with a vector according to claim 1; and
transplanting said transduced cell or cells derived therefrom into said subject where said cells or derivatives therefrom express said anti-sickling human beta globin gene.

60. The method of claim 59, wherein the cell is a stem cell.

61. The host cell of claim 59, wherein said cell is a stem cell derived from bone marrow.

62. The method of claim 59, wherein, wherein the cell is a human hematopoietic progenitor cell.

63. The method of claim 62, wherein the human hematopoietic progenitor cell is a CD34⁺ cell.
